(19) United States
(12) Patent Application Publication
(10) Pub. No.: US 2025/0002518 A1
(43) Pub. Date: Jan. 2, 2025

(54) SYNTHESIS OF HETEROARYNES AND USE THEREOF

(71) Applicant: Regents of the University of Minnesota, Minneapolis, MN (US)

(72) Inventors: Courtney Colleen Mattmiller Roberts, Minneapolis, MN (US); Jenna Nicole Humke, Minneapolis, MN (US); Annabel Quinlivan Ansel, Minneapolis, MN (US); Roman Giancarlo Belli, Minneapolis, MN (US); Sallu So Kargbo, Minneapolis, MN (US); Erin Elizabeth Plasek, Minneapolis, MN (US)

(21) Appl. No.: 18/743,434

(22) Filed: Jun. 14, 2024

Related U.S. Application Data
(60) Provisional application No. 63/521,616, filed on Jun. 16, 2023.

(51) Int. Cl. C07F 15/04; C07D 209/24; C07D 401/04; C07D 471/04; C07D 487/04; C07D 491/048; C07F 5/02; C07F 17/02

(52) U.S. Cl. CPC C07F 15/04; C07D 209/24; C07D 401/04; C07D 471/04; C07D 487/04; C07D 491/048; C07F 5/025; C07F 17/02

(57) ABSTRACT

Disclosed herein are heterocyclic arynes and methods for preparing the same. Heteroarynes such as 5-membered O- or N-heterocyclic arynes have been considered inaccessible due to ring strain associated with the triple bond. The methods described herein demonstrate that these arynes can be successfully prepared through the utility of pi-backbonding from a transition metal. Synthetic utility of this method has been demonstrated by the facile synthesis and functionalization of heterocycles using these heteroaryne building blocks.

### CROSS-REFERENCE TO RELATED APPLICATIONS

This application claims the benefit of U.S. Provisional Patent Application Ser. No. 63/521,616 filed on Jun. 16, 2023, the content of which is incorporated by reference herein in its entirety.

### STATEMENT REGARDING FEDERALLY FUNDED RESEARCH

This invention was made with government support under GM146957 awarded by the National Institutes of Health. The government has certain rights in the invention.

### BACKGROUND

Heteroaromatic compounds are essential building blocks for materials, pharmaceuticals and agrochemicals. In particular, O- and N-heteroaromatics are featured in the scaffolds of a variety of common pharmaceuticals and agrochemicals including Ambien, Atorvastatin and Propyrisulfuron. One promising method for streamlining the synthesis of complex heteroaromatic molecules is via aryne intermediates, due to their ability to undergo mono- or difunctionalization reactions, installing either one or two functional groups on arenes.

Despite its potential in organic synthesis, aryne methodology is afflicted with challenges. For instance, 5-membered O- or N-heterocyclic arynes provide scaffolds to build the essential molecules mentioned above but are underused because five-membered O- and N-heteroarynes have been largely inaccessible on account of the strain of a triple bond in that small of a ring. Thus, there remains a need for methods that allow researchers to synthesize these challenging building blocks.

### SUMMARY

In one aspect, the present disclosure provides a process for the preparation of a heteroaryne of formula (I):

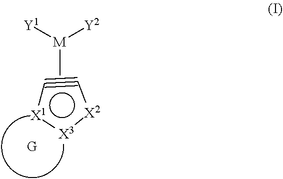

the process comprising the steps of:
(a) converting a compound of formula (II) to a compound of formula (III), and

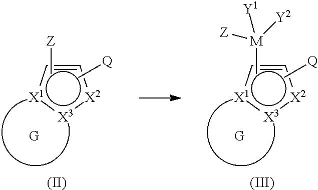

(b) converting the compound of formula (III) to the heteroaryne of formula (I), wherein
X¹, X², and X³ are independently C, CR¹, N, NR², S or O;
R¹ and R² at each occurrence are independently H, alkyl, alkoxy, aryl; —C¹⁻⁴alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR³R⁴R⁵, —SnR³R⁴R⁵, —BF₃Q⁰, —Li or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5- , 6- , or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In another aspect, the present disclosure provides a compound of Formula (II), or a salt thereof:

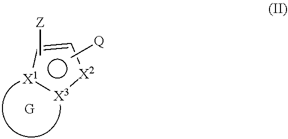

wherein:
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5- , 6- , or 7-membered ring optionally substituted with one or more alkyl groups; and
Z is halogen, —OSO₂R⁶, or —OC(O)R⁶, wherein R⁶ is alkyl, haloalkyl, aryl or amino.

In another aspect, the present disclosure provides a compound of Formula (III), or a salt thereof:

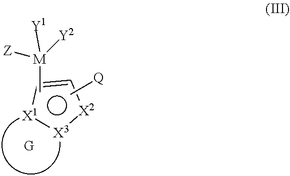

wherein:
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In another aspect, the present disclosure provides a heteroaryne of Formula (I), or a salt thereof:

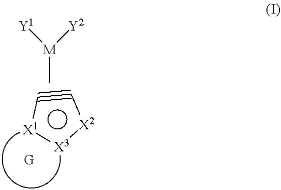

wherein
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In yet another aspect, the present disclosure provides a method for preparing a target molecule, comprising coupling the heteroaryne of Formula (I) as described herein with a partner molecule, thereby producing the target molecule.

### BRIEF DESCRIPTIONS OF THE DRAWINGS

FIG. 1 shows the ORTEP drawing of complex 14 with 50% thermal probability. The complex co-crystallized with benzene. Hydrogen atoms are omitted for clarity.

FIG. 2 shows the ORTEP drawing of complex 15 with 50% thermal probability. The complex co-crystallized with chloroform. Hydrogen atoms are omitted for clarity.

FIG. 3 shows the ORTEP drawing of complex 22 with 50% thermal probability. The complex co-crystallized with dichloromethane. Only the major orientation of disordered groups is shown. Hydrogen atoms and solvent molecules are omitted for clarity. Crystallographic data for compound 3a can be obtained from the Cambridge Crystallographic Data Centre via ccdc.cam.ac.uk, under reference no CCDC 2294627.

FIG. 4 shows the ORTEP drawing of complex 28 with 50% thermal probability. Hydrogen atoms are omitted for clarity.

FIG. 5 shows the ORTEP drawing of complex 31 with 50% thermal probability. Hydrogen atoms are omitted for clarity. Crystallographic data for compound 4a can be obtained from the Cambridge Crystallographic Data Centre via ccdc.cam.ac.uk, under reference no. CCDC 2294631.

FIG. 6 shows the ORTEP drawing of 53 with 50% thermal probability. The complex co-crystallized with DCM. Only the major orientation of disordered groups is shown. Hydrogen atoms and solvent molecules are omitted for clarity. Crystallographic data for compound 3b can be obtained from the Cambridge Crystallographic Data Centre via ccdc.cam.ac.uk, under reference no CCDC 2294628.

FIG. 7 shows the ORTEP drawing of 54 with 50% thermal probability. The complex co-crystallized with DCM. Only the major orientation of disordered groups is shown. Hydrogen atoms and solvent molecules are omitted for clarity. Crystallographic data for compound 3c can be obtained from the Cambridge Crystallographic Data Centre via ccdc.cam.ac.uk, under reference no CCDC 2294629.

FIG. 8 shows the ORTEP drawing of 55 with 50% thermal probability. Hydrogen atoms are omitted for clarity. Crystallographic data for compound 3d can be obtained from the Cambridge Crystallographic Data Centre via ccdc.cam.ac.uk, under reference no CCDC 2295323.

FIG. 9 shows the ORTEP drawing of 56 with 50% thermal probability. Hydrogen atoms are omitted for clarity. Crystallographic data for compound 4b can be obtained from the Cambridge Crystallographic Data Centre via ccdc.cam.ac.uk, under reference no. CCDC 2294630.

FIG. 10 shows the ORTEP drawing of 58 with 50% thermal probability. Hydrogen atoms are omitted for clarity. Crystallographic data for compounds 4d can be obtained from the Cambridge Crystallographic Data Centre via ccdc.cam.ac.uk, under reference no. CCDC 2294632.

FIG. 11 shows (A) ¹H NMR spectrum of 31 compared to (B) 1D ¹H NOESY NMR spectrum (500 MHz, C₆D₆) selectively irradiating the aromatic signal at 8.10 ppm which shows an NOE with a cyclohexyl methine signal at 2.21 ppm. Therefore, the downfield methine peak corresponds to the cyclohexyl ring on the P trans to C1 (highlighted area).

FIG. 12 shows (A) ¹H NMR spectrum of 31 compared to (B) 1D ¹H NOESY NMR spectrum (500 MHz, C₆D₆) selectively irradiating the N-Me signal at 4.17 ppm which shows an NOE with a cyclohexyl methine signal at 2.10 ppm. Therefore, the upfield methine corresponds to the cyclohexyl ring on the P trans to C2 (highlighted area).

FIG. 13 shows the ¹H—¹H NOESY NMR spectrum of complex 31 (500 MHz, C₆D₆). The downfield methine signal at 2.21 ppm shows an NOE correlation with the aromatic proton signal at 8.10 ppm, meaning the downfield methine corresponds to the cyclohexyl ring on the P trans to C1. Additionally, the upfield methine signal at 2.10 ppm shows an NOE correlation with the N-Me signal at 4.17 ppm. Therefore, the upfield methine corresponds to the cyclohexyl ring on the P trans to the C2.

FIG. 14 shows the ¹H—³¹P{¹H} HMBC NMR spectrum of complex 31 (400 MHz, C₆D₆). The downfield ³¹P signal at 91 ppm shows a correlation with the downfield methine signal at 2.21 ppm, therefore the ³¹P signal at 91 ppm corresponds to the P trans to C2. On the other hand, the upfield ³¹P signal at 86 ppm shows a correlation with the upfield methine signal at 2.10 ppm, therefore the ³¹P signal at 86 ppm corresponds to the P trans to C1.

FIG. 15 shows the ¹H-¹³C{¹H} HMBC NMR spectrum of 31 (400 MHz, C₆D₆). The N-Me signal at 4.17 ppm shows a correlation to the ¹³C signal at 166 ppm. Therefore, this signal corresponds to C1. On the other hand, the ¹³C signal at 121 ppm does not show a correlation to the N-Me signal at 4.17 ppm. Therefore, this signal corresponds to C2.

FIG. 16 shows the ³¹P{¹H}—³¹P{¹H} NOESY NMR spectrum of 31 (500 MHz, C₆D₆). No NOE correlation cross peaks were observed which rules out aryne dissociation or rotation on the timescale of the mixing period (3 s).

FIG. 17 shows the UV-Vis spectra of (A) dcpe σ-aryl complex 22 (dashed) compared to aryne complex 31 (solid), (B) dcpe σ-aryl complex 53 (dashed) compared to aryne complex 56 (solid), (C) dcpe σ-aryl complex 54 (dashed) compared to aryne complex 57 (solid), (D) dcpe σ-aryl complex 55 (dashed) compared to aryne complex 58 (solid). All spectra were collected in THF at a concentration of 0.2 mM.

FIG. 18 shows the room temperature solid-state FTIR (KBr) of dcpe σ-aryl complex 22 (dashed) compared to aryne complex 31 (solid).

FIG. 19 shows the image of 7-azaindole and atom coordinates modeled using Spartan V9.

FIG. 20 shows the image of 7-azaindolyne and atom coordinates modeled using Spartan V9.

FIG. 21 shows the ORTEP drawing of 6a with 50% thermal probability. Hydrogen atoms are omitted for clarity. Crystallographic data for compounds 6a can be obtained from the Cambridge Crystallographic Data Centre via ccdc.cam.ac.uk, under reference no. CCDC 2327003.

FIG. 22 shows the ORTEP drawing of 9a with 50% thermal probability. Hydrogen atoms are omitted for clarity. Crystallographic data for compound 9a can be obtained from the Cambridge Crystallographic Data Centre via ccdc.cam.ac.uk, under reference no. CCDC 2327002.

FIG. 23 shows the (A)¹H NMR spectrum of 7a compared to (B) 1D ¹H NOESY NMR spectrum (500 MHz, CDCl₃) selectively irradiating the N-Me proton signal at 3.83 ppm which shows an NOE with C1-H signal at 6.94 ppm. This confirms that 7a is the regioisomer where a Me group has added to C2.

FIG. 24 shows the (A)¹H NMR spectrum of 8a compared to (B) 1D ¹H NOESY NMR spectrum (500 MHz, C₆D₆) selectively irradiating the benzylic proton signal at 6.39 ppm which shows an NOE with a 7-azaindole aromatic signal at 7.48 ppm. This confirms that 8a is the regioisomer where benzaldehyde has added to C2.

FIG. 25 shows the ¹H-¹³C{¹H} HMBC NMR spectrum of compound 8a (500 MHz, C₆D₆) which shows that the N-Me and benzylic protons correlate to the C1 signal. For this spectrum, the NMR parameter cnst13=6 Hz.

FIG. 26 shows the (A) ¹H NMR spectrum of 10a′-CH₂Cy compared to (B) 1D ¹H NOESY NMR spectrum (400 MHz, CDCl₃) selectively irradiating the N-Me signal at 3.79 ppm which shows an NOE with both the methylene signal at 2.67 ppm and cyclohexyl C—H signals at 1.77 and 1.66 ppm. Therefore, the regioisomer formed has the methyl cyclohexyl group at the 2-position of the azaindole ring.

FIG. 27 shows the (A)¹H NMR spectrum of 10a′-CH₂Cy compared to (B) 1D ¹H NOESY NMR spectrum (400 MHz, CDCl₃) selectively irradiating the methylene C—H signal at 2.67 ppm which shows an NOE with N-Me signal at 3.79 ppm. Therefore, the regioisomer formed has the methyl cyclohexyl group at the 2-position of the azaindole ring.

FIG. 28 shows the ¹H-¹³C{¹H} HMBC NMR spectrum of compound 10a-py (500 MHz, CDCl₃).

FIG. 29 shows the ¹H-¹³C{¹H} HMBC NMR spectrum of compound 10a′-py (500 MHz, CDCl₃).

FIG. 30 shows the (A)¹H NMR spectrum of 11a compared to (B) 1D ¹H NOESY NMR spectrum (500 MHz, CDCl₃) selectively irradiating the N-Me signal at 3.88 ppm which only shows an NOE with the aryl methyl signal at 2.55 ppm. This confirms that this isolated product is 11a.

FIG. 31 shows the (A)¹H NMR spectrum of 11a′ compared to (B) 1D ¹H NOESY NMR spectrum (400 MHz, CDCl₃) selectively irradiating the N-Me signal at 3.49 ppm which only shows an NOE with the mesityl methyl signal at 1.99 ppm. This confirms that this isolated product is 11a′.

FIG. 32 shows the synthesis and accessibility of arynes and orbital interactions of metal π ligands. (A) Formation and accessibility of arynes through elimination. (B) Hypothesis of this work. [M] represents the transition metal complex. (C) Metal-π ligand interactions. (D) Comparison of computed and crystallographic bond lengths of benzene and benzyne. (E) Formation of Ni-benzyne. OTf, trifluoromethanesulfonate; LG, leaving group; M, metal; L, ligand; pin, pinacol; OA, oxidative addition; TM, transmetalation.

FIG. 33 shows the synthetic access to Ni-coordinated 7-azaindolynes. (A) Synthesis of heteroaryne precursors. (B) Development of a route to Ni-bound 7-azaindolyne complex. See Example 3 for reaction conditions. (C) Electronically and sterically differentiated Ni-bound 7-azaindolyne complexes. LDA, lithium diisopropylamide; Bn, benzyl; COD, 1,5-cyclooctadiene; O'Pent, tert-pentoxide.

FIG. 34 shows structural and spectroscopic characterization. (A) Comparison of computed and crystallographic bond lengths of 7-azaindole, 22, 31, and 7-azaindolyne. (B) Crystallographic comparison of electronically and sterically differentiated Ni-bound 7-azaindolyne complexes. (C)³¹P{¹H} NMR spectra of 31, 56-58 in THF-d₈. (D) UV-vis spectra of 22, 53-55 compared with that of 31, 56-58, 0.2 mM in THF.

FIG. 35 shows the studies of the transmetalation step during aryne formation. (A) Proposed pathway for transmetalation through an activated borate intermediate. (B) Reaction monitoring through ³¹P{¹H} and ¹¹B NMR spectroscopy in THF. *, unidentified borate species.

FIG. 36 shows the reactivity of Ni-bound 7-azaindolyne. (A) Reactivity of 31 with electrophilic and nucleophilic sources of methyl. (B) Underexplored classes of electrophilic coupling partners. (C) C(sp³), C(sp²), and C(sp) hybridized nucleophilic coupling partners. (D) Difunctionalization reactions that use aryne and σ-aryl intermediates to generate two regioisomers. (E) Annulation of 31 with alkyne coupling partners. R.r.'s are of functionalization at C2:C1; major regioisomers are depicted. Unless noted, all yields were calculated by ¹H NMR spectroscopy with a mesitylene or CH₂Br₂ internal standard. *, isolated yield; OTFA=trifluoroacetate; Mes=mesityl.

FIG. 37 illustrates the access to aryne of 7-azaindol-2,3-yne through alleviation of strain through metal binding, and expansion of the scope to other heteroaryne scaffolds.

FIG. 38 shows the synthesis of precursors for various heteroaryne scaffolds.

FIG. 39 shows A) synthesis of heteroaryne complexes through 2 routes, and B) borate formation with 4-azaindole.

FIG. 40 shows functionalization of the heteroarynes.

FIG. 41 shows the various ligands used during the formation of heteroarynes.

FIG. 42 shows the ORTEP drawing of 70 with 50% thermal probability. Hydrogen atoms are omitted for clarity. Only the major orientation of disordered groups is shown.

FIG. 43 shows the ORTEP drawing of 77 with 50% thermal probability. Hydrogen atoms and solvent molecules are omitted for clarity. Only the major orientation of the disorder groups is shown.

FIG. 44 shows the ORTEP drawing of 80 with 50% thermal probability. Hydrogen atoms are omitted for clarity.

FIG. 45 shows (A)¹H NMR spectrum of 13a compared to (B) 1D ¹H NOESY NMR spectrum (500 MHz, CDCl₃) selectively irradiating the C-Me proton signal at 2.61 ppm which does not show an NOE with the N-Me signal at 3.75 ppm This confirms that 13b is the regioisomer where a Me group has added to C2.

FIG. 46 shows (A)¹H NMR spectrum of 13b compared to (B) 1D ¹H NOESY NMR spectrum (500 MHz, CDCl₃) selectively irradiating the C-Me proton signal at 2.41 ppm which does not show an NOE with the N-Me signal at 3.80 ppm This confirms that 13b is the regioisomer where a Me group has added to C2.

FIG. 47 shows (A)¹H NMR spectrum of 14b compared to (B) 1D ¹H NOESY NMR spectrum (500 MHz, CDCl₃) selectively irradiating the C-Me proton signal at 2.42 ppm which does not show an NOE with the N—CH₂-Ph signal at 5.56 ppm This confirms that 14b is the regioisomer where a Me group has added to C2.

### DETAILED DESCRIPTION

O- and N-heteroarynes provide a scaffold as building blocks for many essential molecules but are underused due to their inaccessibility. On the basis of principles of metal-ligand interactions that are foundational to organometallic chemistry, stabilization of O- and N-heteroarynes in the transition metal coordination sphere and a series of heteroaryne complexes synthesized and characterized crystallographically and spectroscopically are disclosed herein. Ambiphilic reactivity of the heteroaryne complexes was observed with multiple nucleophilic, electrophilic, and enophilic coupling partners. See Humke et al., Nickel binding enables isolation and reactivity of previously inaccessible 7-aza-2,3-indolynes, Science, 384, 408-414(2024). DOI:10.1126/science.adil606. The contents of which are incorporated here by reference in its entirety.

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched chain saturated hydrocarbon. The alkyl can be a C₁₋₄alkyl. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkoxy” as used herein, refers to an alkyl group, as defined herein, appended to the parent molecular moiety through an oxygen atom. The alkoxy can be a C₁₋₄alkoxy. Representative examples of alkoxy include, but are not limited to, methoxy, ethoxy, propoxy, 2-propoxy, butoxy and tert-butoxy.

The term “alkylene,” as used herein, means a divalent group derived from a straight or branched chain saturated hydrocarbon. Representative examples of alkylene include, but are not limited to, —CH₂—, —CH₂CH₂—, —CH₂CH₂CH₂—, —CH₂CH(CH₃)CH₂—, and CH₂CH(CH₃)CH(CH₃)CH₂—.

The term “aryl,” as used herein, means phenyl or a bicyclic aryl. The bicyclic aryl can be a phenyl fused to a cycloalkyl moiety. Examples of aryl include naphthyl, dihydronaphthalenyl, tetrahydronaphthalenyl, indanyl, or indenyl. The phenyl and bicyclic aryls are attached to the parent molecular moiety through any carbon atom contained within the phenyl or bicyclic aryl.

The term “cycloalkyl” as used herein, means a monovalent group derived from an all-carbon ring system containing zero heteroatoms as ring atoms, and zero double bonds. The all-carbon ring system can be a monocyclic, bicylic, or tricyclic ring system, and can be a fused ring system, a bridged ring system, or a spiro ring system, or combinations thereof. Examples of cycloalkyls include, but are not limited to, cyclopropyl, cyclobutyl, cyclopentyl, cyclohexyl, cycloheptyl, cyclooctyl, and

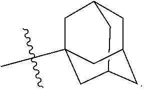 [SMILES: *C12CC3CC(CC(C3)C1)C2]

The cycloalkyl groups described herein can be appended to the parent molecular moiety through any substitutable carbon atom. As used herein, “Cy” means cyclohexyl.

The term “halogen” or “halo” means a chlorine, bromine, iodine, or fluorine atom.

A “ligand” as used herein refers to an organic group or moiety that form a complex with a metal. Representative examples of ligands include, but are not limited to, nitrogen-based ligands such as ethylenediamine, or bidentate-phosphine ligands such as dicyclohexylphosphinoethane (dcpe) and triphenylphoshine (PPh₃).

The terms “amino” and “amine” include both unsubstituted and substituted amines (e.g., mono-substituted amines or di-substituted amines), wherein substituents may include, for example, alkyl, alkoxy, silyl, carbonyl, sulfonyl, cycloalkyl, heterocycle, and aryl as defined herein).

The term “salt” means as used herein refers to acid addition salts and basic addition salts. It may also refer to those salts that may be prepared in situ during the isolation and purification of the compounds disclosed herein. Acid addition salts are salts of an amino group formed with inorganic acids (such as hydrochloric acid, hydrobromic acid, phosphoric acid, sulfuric acid and perchloric acid) or with organic acids (such as acetic acid, oxalic acid, maleic acid, tartaric acid, citric acid, succinic acid or malonic acid) or by using other methods used in the art such as ion exchange. Other examples of salts include adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C₁₋₄alkyl)₄ salts. This also includes the quaternization of any basic nitrogen-containing groups of the compounds disclosed herein. Representative alkali or alkaline earth metal salts include sodium, lithium, potassium, calcium, magnesium, and the like.

The term “nitro” means —NO₂.

The term “cyano” means —CN.

The term “haloalkyl,” as used herein, means an alkyl, as defined herein, in which one, two, three, four, five, six, or seven hydrogen atoms are replaced by halogen. For example, representative examples of haloalkyl include, but are not limited to, 2-fluoroethyl, difluoromethyl, trifluoromethyl, 2,2,2-trifluoroethyl, 2,2,2-trifluoro-1, 1-dimethylethyl, and the like.

The term “heteroaryl” as used herein, means an aromatic heterocycle, i.e., an aromatic ring that contains at least one heteroatom selected from O, N, or S. A heteroaryl may contain from 5 to 12 ring atoms. A heteroaryl may be a 5- to 6-membered monocyclic heteroaryl or an 8- to 12-membered bicyclic heteroaryl. A 5-membered monocyclic heteroaryl ring contains two double bonds, and one, two, three, or four heteroatoms as ring atoms. Representative examples of 5-membered monocyclic heteroaryls include, but are not limited to, furanyl, imidazolyl, isoxazolyl, isothiazolyl, oxadiazolyl, oxazolyl, pyrazolyl, pyrrolyl, tetrazolyl, thiadiazolyl, thiazolyl, thienyl, and triazolyl. A 6-membered heteroaryl ring contains three double bonds, and one, two, three or four heteroatoms as ring atoms. Representative examples of 6-membered monocyclic heteroaryls include, but are not limited to, pyridinyl, pyridazinyl, pyrimidinyl, pyrazinyl, and triazinyl. The bicyclic heteroaryl is an 8- to 12-membered ring system having a monocyclic heteroaryl fused to an aromatic, saturated, or partially saturated carbocyclic ring, or fused to a second monocyclic heteroaryl ring. Representative examples of bicyclic heteroaryl include, but are not limited to, benzofuranyl, benzoxadiazolyl, 1,3-benzothiazolyl, benzimidazolyl, benzothienyl, indolyl, indazolyl, isoquinolinyl, naphthyridinyl, oxazolopyridine, quinolinyl, thienopyridinyl, 5,6, 7,8-tetrahydroquinolinyl, and 6, 7-dihydro-5H-cyclopenta[b Jpyridinyl. The heteroaryl groups are connected to the parent molecular moiety through any substitutable carbon atom or any substitutable nitrogen atom contained within the groups.

The term “heteroaryne” as used herein, means a heteroaryl compound having a carbon-carbon triple bond. The term “heteroaryne” can further refer to a complex comprising a heteroaryl compound with a carbon-carbon triple bond bound to a transition metal as a metallacyclopropene with two X-type donors, or a complex comprising a heteroaryl compound with a carbon-carbon triple bond coordinated to a transition metal like an alkyne (i.e., L-type donor).

The terms “heterocycle” or “heterocyclic” refer generally to ring systems containing at least one heteroatom as a ring atom where the heteroatom is selected from oxygen, nitrogen, and sulfur. In some embodiments, a nitrogen or sulfur atom of the heterocycle is optionally substituted with oxo. Heterocycles may be a monocyclic heterocycle, a fused bicyclic heterocycle, or a spiro heterocycle. The monocyclic heterocycle is generally a 4, 5, 6, 7, or 8-membered non-aromatic ring containing at least one heteroatom selected from O, N, or S. The 4-membered ring contains one heteroatom and optionally one double bond. The 5-membered ring contains zero or one double bond and one, two or three heteroatoms. The 6, 7, or 8-membered ring contains zero, one, or two double bonds, and one, two, or three heteroatoms. Representative examples of monocyclic heterocycle include, but are not limited to, azetidinyl, azepanyl, diazepanyl, 1,3-dioxanyl, 1,4-dioxanyl, 1,3-dioxolanyl, 4,5-dihydroisoxazol-5-yl, 3,4-dihydropyranyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl, thiopyranyl, and trithianyl. The fused bicyclic heterocycle is a 7-12-membered ring system having a monocyclic heterocycle fused to a phenyl, to a saturated or partially saturated carbocyclic ring, or to another monocyclic heterocyclic ring, or to a monocyclic heteroaryl ring. Representative examples of fused bicyclic heterocycle include, but are not limited to, 1,3-benzodioxol-4-yl, 1,3-benzodithiolyl, 3-azabicyclo[3.1.0]hexanyl, hexahydro-1H-furo[3,4-c]pyrrolyl, 2,3-dihydro-1,4-benzodioxinyl, 2,3-dihydro-1-benzofuranyl, 2,3-dihydro-1-benzothienyl, 2,3-dihydro-1H-indolyl, 5,6,7,8-tetrahydroimidazo[1,2-a]pyrazinyl, and 1,2,3,4-tetrahydroquinolinyl. Spiro heterocycle means a 4-, 5-, 6-, 7-, or 8-membered monocyclic heterocycle ring wherein two of the substituents on the same carbon atom form a second ring having 3, 4, 5, 6, 7, or 8 members. Examples of a spiro heterocycle include, but are not limited to, 1,4-dioxa-8-azaspiro[4.5]decanyl, 2-oxa-7-azaspiro[3.5]nonanyl, 2-oxa-6-azaspiro[3.3]heptanyl, and 8-azaspiro[4.5]decane. The monocyclic heterocycle groups of the present invention may contain an alkylene bridge of 1, 2, or 3 carbon atoms, linking two nonadjacent atoms of the group. Examples of such a bridged heterocycle include, but are not limited to, 2,5-diazabicyclo[2.2.1]heptanyl, 2-azabicyclo[2.2.1]heptanyl, 2-azabicyclo[2.2.2]octanyl, and oxabicyclo[2.2.1]heptanyl. The monocyclic, fused bicyclic, and spiro heterocycle groups are connected to the parent molecular moiety through any substitutable carbon atom or any substitutable nitrogen atom contained within the group.

The term “alkylene-aryl” as used herein refers to an aryl, as defined herein, in which a hydrogen atom is replaced by an alkylene group. For example, representative examples of alkylene-aryl include, but are not limited to, C₁₋₄alkylene-aryls, such as —CH₂Ph (Bn group), —CH₂CH₂Ph, —CH₂CH₂CH₂Ph, —CH₂CH₂CH₂CH₂Ph, and the like.

The term “metallocene” as used herein refers to a compound comprising two cyclopentadienyl anions bound to a metal center. Suitable metals include, but are not limited to, iron, cobalt, chromium, nickel, and vanadium. In some embodiments, the metal center is iron (Fe) and the metallocene is ferrocene:

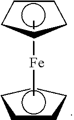

In some embodiments, the ferrocene may be attached to one or more compounds with the connectivity shown here:

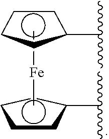

The term “hydroxy” as used herein, means an —OH group.

The term hydroxyalkyl as used herein means an alkyl, as defined herein, in which a hydrogen atom is replaced by —OH. For example, representative examples of hydroxyalkyl include, but are not limited to those derived from C₁₋₆ alkyls, such as —CH₂OH, —CH₂CH₂OH, —CH₂CH₂CH₂OH, and the like.

The term “oxo” as used herein refers to an oxygen atom bonded to the parent molecular moiety. An oxo may be attached to a carbon atom or a sulfur atom by a double bond. Alternatively, an oxo may be attached to a nitrogen atom by a single bond, i.e., an N-oxide.

Terms such as “alkyl”, “cycloalkyl”, “alkylene”, “cycloalkylene”, “polycycloalkylidene” etc. may be preceded by a designation indicating the number of atoms present in the group in a particular instance (e.g., “C₁-C₄alkyl”, “C₁₋₄alkyl”, “C₃₋₆cycloalkyl”, “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄,” the members of the group that follows may have any number of carbon atoms falling within the recited range. A “C₁-C₄alkyl” or “C₁₋₄alkyl”, for example, is an alkyl group having from 1 to 4 carbon atoms, however arranged (i.e., straight chain or branched).

If a group is described as being “substituted”, a non-hydrogen substituent group is in the place of hydrogen radical on a carbon or nitrogen of that group. Thus, for example, a substituted alkyl is an alkyl in which at least one non-hydrogen radical is in the place of a hydrogen radical on the alkyl. To illustrate, monofluoroalkyl is alkyl substituted with a fluoro radical, and difluoroalkyl is alkyl substituted with two fluoro radicals. It should be recognized that if there is more than one substitution on a substituent, each non-hydrogen radical may be identical or different (unless otherwise stated). Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

When a group is referred to as “unsubstituted” or not referred to as “substituted” or “optionally substituted”, it means that the group does not have any substituents. If a group is described as being “optionally substituted”, the group may be either (1) not substituted or (2) substituted. If a group is described as being optionally substituted with up to a particular number of non-hydrogen radicals, that group may be either (1) not substituted; or (2) substituted by up to that particular number of substituent groups or by up to the maximum number of substitutable positions on that group, whichever is less.

If substituents are described as being independently selected from a group, each substituent is selected independent of the other. Each substituent, therefore, may be identical to or different from the other substituent(s).

Unless otherwise stated, structures depicted herein are also meant to include all isomeric (e.g., enantiomeric, diastereomeric, regioisomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. The compound disclosed herein may exist as a regioisomer or a mixture of regioisomers. Unless otherwise stated, all tautomeric and regioisomeric forms of the compounds of the invention are within the scope of the invention.

The present disclosure generally relates to a method of synthesizing heteroarynes such as 5-membered O- or N-heterocyclic arynes, which provides an approach to these synthetically challenging compounds. In various embodiments, heteroarynes have been accessed, serving as important precursors to valuable molecules useful as pharmaceutical and agrochemical candidates. Utility of these heteroarynes have been demonstrated by successful coupling with various partner molecules.

### Process

In one aspect, the present disclosure provides a process for the preparation of a heteroaryne of formula (I)

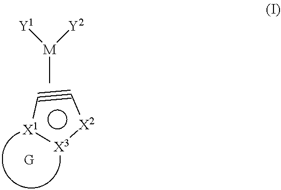

the process comprising the steps of:
(a) converting a compound of formula (II) to a compound of formula (III), and

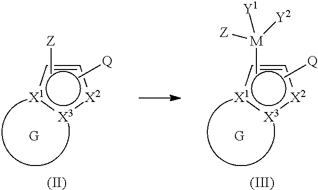

(b) converting the compound of formula (III) to the heteroaryne of formula (I), wherein
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR³R⁴R⁵, —SnR³R⁴R⁵, —BF₃Q⁰, —Li or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In some embodiments, formula (II) has a structure of formula II(a). For example, the process can comprise the steps of:
(a) converting the compound of formula II(a) to a compound of formula III(a), and

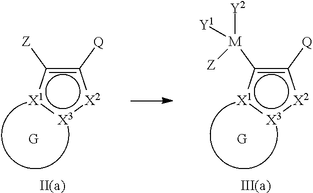

(b) converting the compound of formula III(a) to the heteroaryne of formula (I).

In some embodiments, formula (I) has a structure of formula I(a-1),

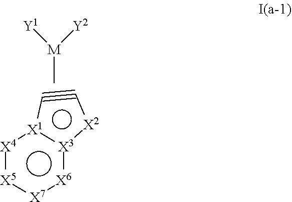

and formula II(a) has a structure of formula II(a-1). For example, the process can comprise the steps of:
(a) converting the compound of formula II(a-1) to a compound of formula III(a-1), and

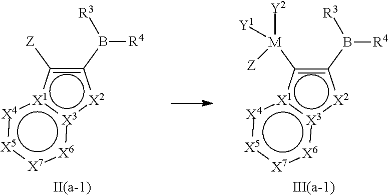

(b) converting the compound of formula III(a-1) to the heteroaryne of formula I(a-1), wherein X⁴, X⁵, X⁶, and X⁷ are independently CR¹⁰ or N, and R¹⁰ at each occurrence is independently H, alkyl, halo, alkoxy, or haloalkyl. The other groups are the same as defined herein. For example, X⁴, X⁵, X⁶, and X⁷ are independently CH or N.

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(iii) X¹ is C, X² is O, X³ is C, X⁴ is CR¹⁰ X⁵ is CR¹⁰, and X⁶ is N,
(iv) X¹ is C, X² is NR², X³ is C, X⁴ is N, X⁵ is CR¹⁰, and X⁶ is N,
(v) X¹ is N, X² is N, X³ is C, X⁴ is CR¹⁰, X⁵ is CR¹⁰, and X⁶ is CR¹⁰,
(vi) X¹ is C, X² is O, X³ is C, X⁴ is CR¹⁰, X⁵ is CR¹⁰ and X⁶ is CR¹⁰,
(vii) X¹ is C, X² is NR², X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰,
(ix) X¹ is C, X² is O, X³ is C, X⁴ is N, X⁵ is CR¹⁰, and X⁶ is CR¹⁰,
(x) X¹ is C, X² is N, X³ is N, X⁴ is CR¹⁰, X⁵ is CR¹⁰, and X⁶ is CR¹⁰,
(xi) X¹ is C, X² is CH, X³ is N, X⁴ is CR¹⁰, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰;
or X⁷ is N, and
(xiii) X¹ is C, X² is NR², X³ is C, X⁴ is CR¹⁰, X⁵ is CR¹⁰, and X⁶ is CR¹⁰.

In some such embodiments, X⁴, X⁵, X⁶, and X⁷ are independently CH or N.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH.

In some embodiments, X⁷ is N, and
(xiii) X¹ is C, X² is NR², X³ is C, X⁴ is CH, X⁵ is CH, and X⁶ is CH.

For example, in these structures, R² can be H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A}. R^{A}, R^{B}, and R^{C} at each occurrence are independently H, alkyl, haloalkyl, or aryl. In some embodiments, R² is methyl. In some embodiments, R² is benzyl (Bn).

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(vi) X¹ is C, X² is O, X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH
(viii) X¹ is C, X² is NR², X³ is C, X⁴ is N, X⁵ is CH, and X⁶ is CH, or
(xii) X¹ is C, X² is NR², X³ is C, X⁴ is CH, X⁵ is N, and X⁶ is CH.

G may or may not contain heteroatoms. In some embodiments, G is a 6-membered ring. In some embodiments, G is a phenyl ring. In some embodiments, G is a pyridine. In some embodiments, G is a pyrimidine. In some embodiments, G is a pyrazine. In some embodiments, G is substituted with one or more alkoxy, such as methoxy. In some embodiments, G is substituted with one or more alkyl, such as methyl. In some embodiments, G is substituted with one or more haloalkyl, such as —CF₃.

In some embodiments, Q⁰ is a metal counter ion, such as sodium, potassium, or lithium.

In some embodiments, Z is halogen. In some embodiments, Z is a group commonly understood as “pseudo halogen”, such as sulfonate, sulfamate, or ester. In some embodiments, Z is bromide, —OSO₂CF₃, —OSO₂Ph, —OSO₂Me, —OSO₂Nme₂, —OC(O)Me, or —OC(O)tBu.

In some embodiments, Q is —BR³R⁴, wherein R³ and R⁴ are independently hydroxy or alkoxy, or R³ and R⁴ together with the boron that they are attached to form a 5-, 6-, or 7-membered ring optionally substituted with one or more alkyl groups. In some particular embodiments, Q is

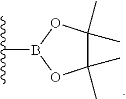 [SMILES: *B1OC(C)(C)C(C)(C)O1]

In some embodiments, M is transition metal. Transition metal includes chemical elements in the d-block of the periodic table (groups 3-12). In some embodiments, M is nickel, palladium, or zirconium.

In some embodiments, R⁷ is cyclohexyl or phenyl. In some embodiments, R⁷ is cyclohexyl. In some embodiments, R⁷ is phenyl. In some embodiments, R⁷ is phenyl and additional ligand exchange steps may be needed before converting the compound of formula (III), III(a), or III(a-1) to the heteroaryne of formula (I), I(a), or I(a-1).

In some embodiments, n is 2 or 3.

In some embodiments, -M(Y¹)(Y²) is

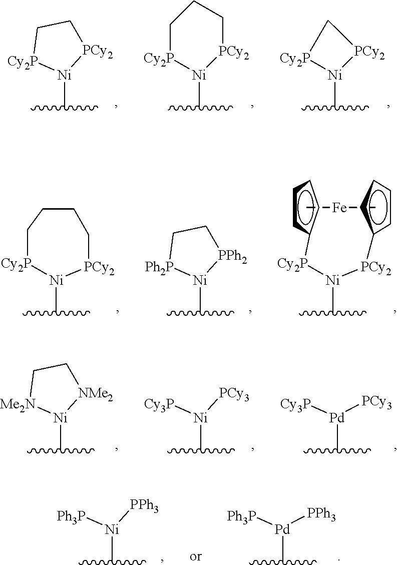

In some embodiments, -M(Y¹)(Y²) is

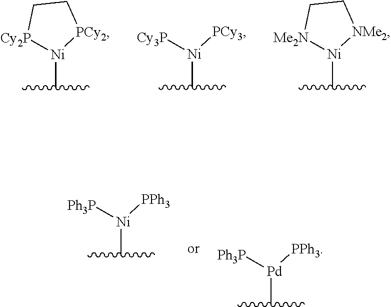

In some embodiments, -M(Y¹)(Y²) is

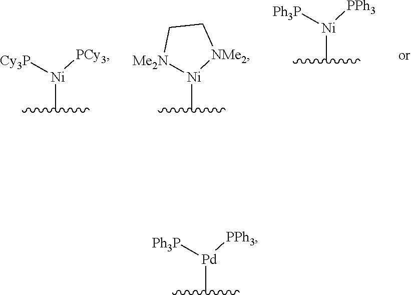

and additional ligand exchange steps may be needed before converting the compound of formula (III), III(a), or III(a-1) to the heteroaryne of formula (I), I(a), or I(a-1).

In some embodiments, the compound of formula (II), formula II(a), or formula II(a-1) is

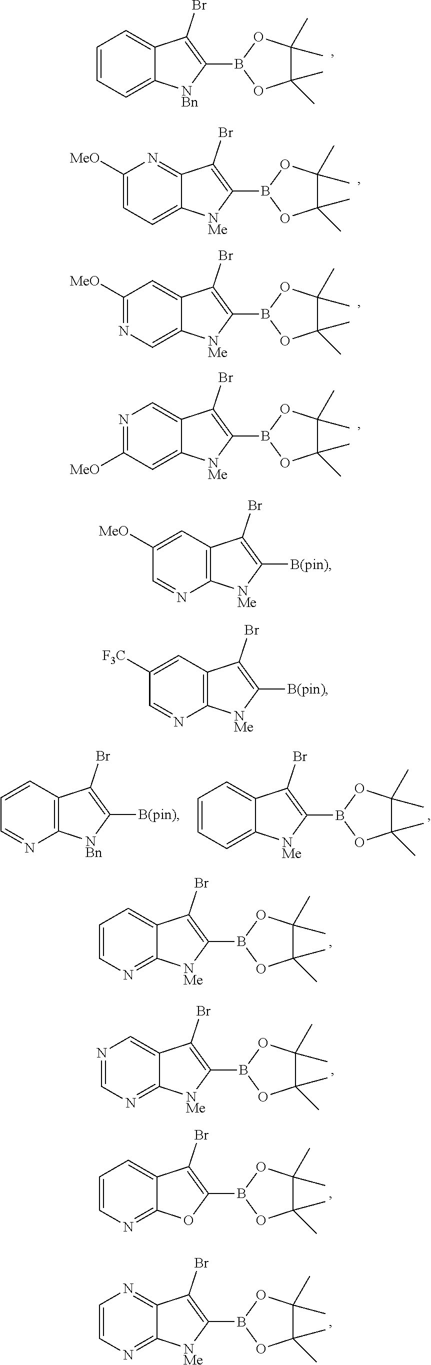

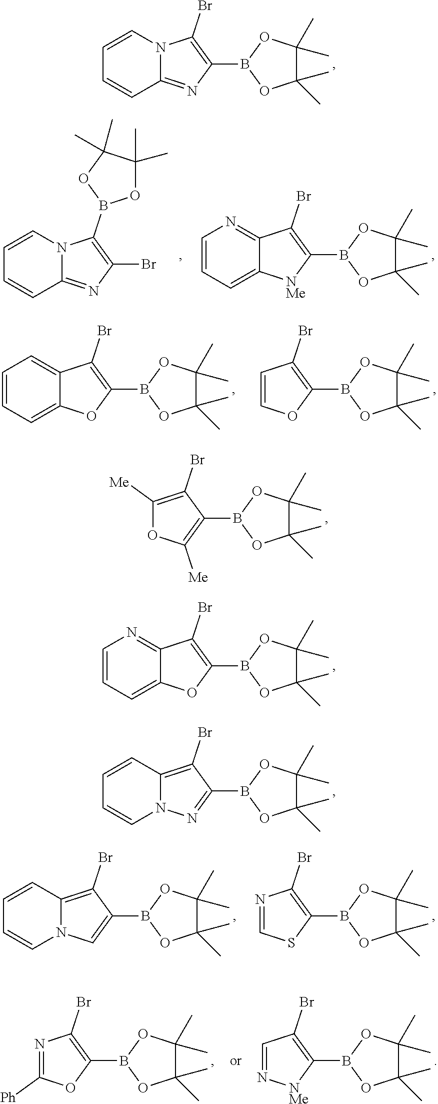

In some embodiments, step (a) is carried out in the presence of a complex comprising the transition metal M and a ligand. Representative examples of such complex include, but are not limited to, organometallic compounds such as bis(1,5-cyclooctadiene)nickel(0) (CAS registry number 1295-35-8) and bis(dibenzylideneacetone)palladium(0) (CAS registry number 32005-36-0). In some embodiments, the complex is bis(1,5-cyclooctadiene)nickel(0). In some embodiments, the complex is bis(Di benzylideneacetone)palladium(0).

The ligand can be used, for example, to form a complex with the transition metal M in formula (III). In some embodiments, M can be introduced in step (a) as a complex with a ligand (e.g., bis(cyclooctadiene)). For example, the M(Y¹)(Y²) moiety of formula (III) is formed by a reaction in which the (M-ligand) complex is used. In some embodiments, Y¹ and Y² are cyclohexyl or phenyl, and the M(Y¹)(Y²) moiety is formed by the reaction in which the (M-ligand) complex is used. In some embodiments, formula (III) needs to go through a ligand exchange reaction as described herein before being converted to formula (I). Such ligand exchange reaction may include, for example, converting a phenyl-containing ligand (e.g., PPh₃) to a cyclohexyl-containing ligand (e.g., —P(Cy)₂-CH₂CH₂—P(Cy)₂-). Suitable ligands include nitrogen-based ligands such as ethylenediamine, and bidentate-phosphine ligands such as dicyclohexylphosphinoethane (dcpe) and triphenylphoshine (PPh₃). In some embodiments, the process is carried out in the presence of dicyclohexylphosphinoethane. In some embodiments, the process is carried out in the presence of triphenylphosphine, followed by ligand exchange in the presence of dicyclohexylphosphinoethane.

In some embodiments, step (b) is carried out in the presence of a base. Suitable bases include known reagents useful as a base in organic synthesis. In some embodiments, the base is a tert-butoxide, a tert-pentoxide, or a fluoride. For example, the base can be potassium tert-butoxide, potassium tert-pentoxide, or potassium fluoride.

In some embodiments, the process further comprises converting a compound of formula (IV) to the compound of formula (II).

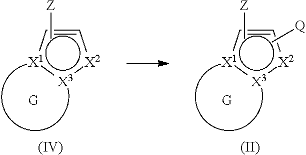

In some embodiments, formula (IV) has a structure of formula IV(a). For example, the process can comprise converting a compound of formula IV(a) to the compound of formula II(a).

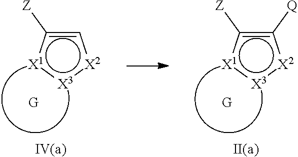

In some embodiments, formula IV(a) has a structure of formula IV(a-1). For example, the process can comprise converting a compound of formula IV(a-1) to the compound of formula II(a-1).

The process disclosed herein may be carried out on a variety of scales and is suitable for scaling up. For example, the process may be carried out on a milligram scale, a gram scale, or a kilogram scale.

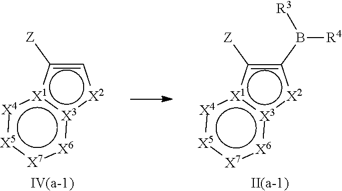

### Compounds

In another aspect, the present disclosure provides a compound of formula (II), or a salt thereof, which may be useful for the present process. In particular, the present disclosure provides a compound of formula (II), or a salt thereof,

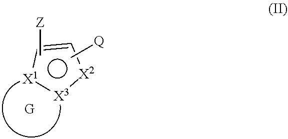

wherein:
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups; and
Z is halogen, —OSO₂R⁶, or —OC(O)R⁶, wherein R⁶ is alkyl, haloalkyl, aryl or amino.

In some embodiments, formula (II) has a structure of formula II(a).

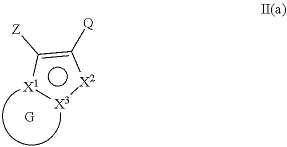

In some embodiments, formula II(a) has a structure of formula II(a-1)

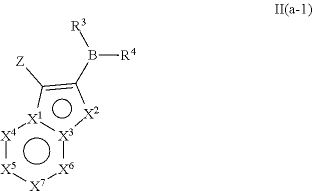

wherein X⁴, X⁵, X⁶, and X⁷ are independently CR¹⁰ or N, and R¹⁰ at each occurrence is independently H, alkyl, halo, alkoxy, or haloalkyl. The other groups are the same as defined herein. For example, X⁴, X⁵, X⁶, and X⁷ are independently CH or N.

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(iii) X¹ is C, X² is O, X³ is C, X⁴ is CR¹⁰ X⁵ is CR¹⁰, and X⁶ is N,
(iv) X¹ is C, X² is NR², X³ is C, X⁴ is N, X⁵ is CR¹⁰, and X⁶ is N,
(v) X¹ is N, X² is N, X³ is C, X⁴ is CR¹⁰, X⁵ is CR¹⁰, and X⁶ is CR¹⁰,
(vi) X¹ is C, X² is O, X³ is C, X⁴ is CR¹⁰, X⁵ is CR¹⁰ and X⁶ is CR¹⁰,
(vii) X¹ is C, X² is NR², X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰,
(ix) X¹ is C, X² is O, X³ is C, X⁴ is N, X⁵ is CR¹⁰, and X⁶ is CR¹⁰,
(x) X¹ is C, X² is N, X³ is N, X⁴ is CR¹⁰, X⁵ is CR¹⁰, and X⁶ is CR¹⁰,
(xi) X¹ is C, X² is CH, X³ is N, X⁴ is CR¹⁰, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.
or X⁷ is N, and
(xiii) X¹ is C, X² is NR², X³ is C, X⁴ is CR¹⁰, X⁵ is CR¹⁰, and X⁶ is CR¹⁰.

In some such embodiments, X⁴, X⁵, X⁶, and X⁷ are independently CH or N.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH.

In some embodiments, X⁷ is N, and
(xiii) X¹ is C, X² is NR², X³ is C, X⁴ is CH, X⁵ is CH, and X⁶ is CH.

For example, in these structures, R² can be H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A}. R^{A}, R^{B}, and R^{C} at each occurrence are independently H, alkyl, haloalkyl, or aryl. In some embodiments, R² is methyl. In some embodiments, R² is benzyl (Bn).

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(vi) X¹ is C, X² is O, X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH
(viii) X¹ is C, X² is NR², X³ is C, X⁴ is N, X⁵ is CH, and X⁶ is CH, or
(xii) X¹ is C, X² is NR², X³ is C, X⁴ is CH, X⁵ is N, and X⁶ is CH.

G may or may not contain heteroatoms. In some embodiments, G is a 6-membered ring. In some embodiments, G is a phenyl ring. In some embodiments, G is a pyridine. In some embodiments, G is a pyrimidine. In some embodiments, G is a pyrazine. In some embodiments, G is substituted with one or more alkoxy, such as methoxy. In some embodiments, G is substituted with one or more alkyl, such as methyl. In some embodiments, G is substituted with one or more haloalkyl, such as —CF₃.

In some embodiments, Q⁰ is a metal counter ion, such as sodium, potassium, or lithium.

In some embodiments, Z is halogen. In some embodiments, Z is a group commonly understood as “pseudo halogen”, such as sulfonate, sulfamate, or ester. In some embodiments, Z is bromide, —OSO₂CF₃, —OSO₂Ph, —OSO₂Me, —OSO₂Nme₂, —OC(O)Me, or —OC(O)tBu.

In some embodiments, Q is —BR³R⁴, wherein R³ and R⁴ are independently hydroxy or alkoxy, or R³ and R⁴ together with the boron that they are attached to form a 5-, 6-, or 7-membered ring optionally substituted with one or more alkyl groups. In some particular embodiments, Q is

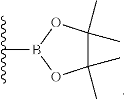 [SMILES: *B1OC(C)(C)C(C)(C)O1]

In some embodiments, the compound of formula (II), II(a), or II(a-1) is

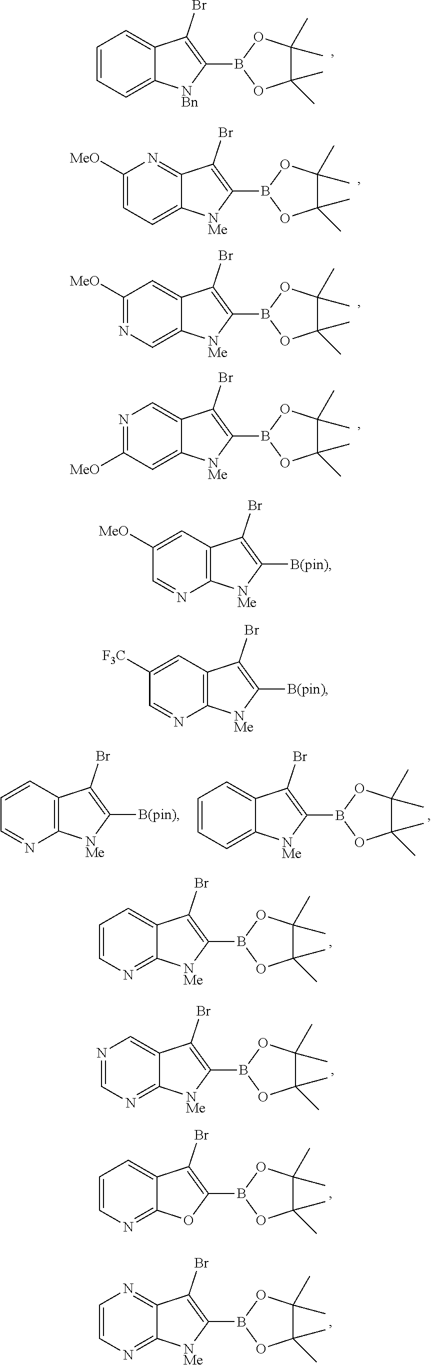

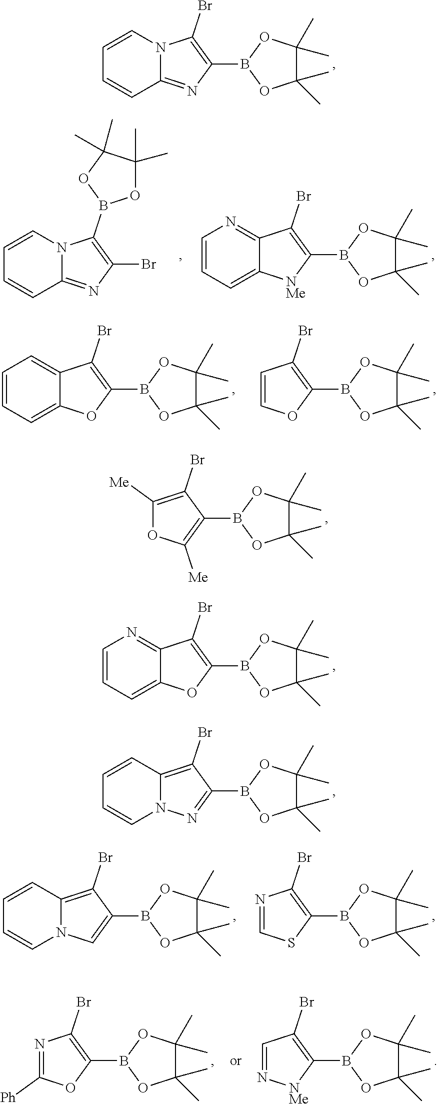

In another aspect, the present disclosure provides a compound of formula (III), or a salt thereof, which may be useful for and produced during the present process. In particular, the present disclosure provides a compound of formula (III), or a salt thereof

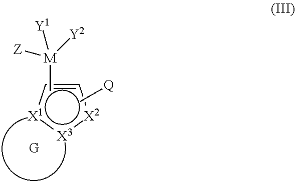

wherein:
X¹, X², and X³ are independently C, CR¹, N, NR², S or O;
R¹ and R² at each occurrence are independently H, alkyl, alkoxy, aryl; —C₁₋₄ alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li or —Ag,
wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In some embodiments, formula (III) has a structure of formula III(a).

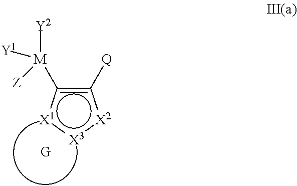

In some embodiments, formula III(a) has a structure of formula III(a-1).

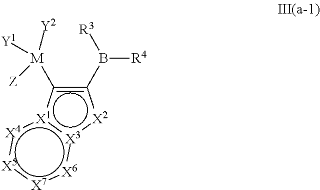

wherein X⁴, X⁵, X⁶, and X⁷ are independently CR¹⁰ or N, and R¹⁰ at each occurrence is independently H, alkyl, halo, alkoxy, or haloalkyl. The other groups are the same as defined herein. For example, X⁴, X⁵, X⁶, and X⁷ are independently CH or N.

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(iii) X¹ is C, X² is O, X³ is C, X⁴ is CR¹⁰ X⁵ is CR¹⁰, and X⁶ is N,
(iv) X¹ is C, X² is NR², X³ is C, X⁴ is N, X⁵ is CR¹⁰, and X⁶ is N,
(v) X¹ is N, X² is N, X³ is C, X⁴ is CR¹⁰, X⁵ is CR¹⁰, and X⁶ is CR¹⁰,
(vi) X¹ is C, X² is O, X³ is C, X⁴ is CR¹⁰, X⁵ is CR¹⁰ and X⁶ is CR¹⁰,
(vii) X¹ is C, X² is NR², X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰,
(ix) X¹ is C, X² is O, X³ is C, X⁴ is N, X⁵ is CR¹⁰, and X⁶ is CR¹⁰,
(x) X¹ is C, X² is N, X³ is N, X⁴ is CR¹⁰, X⁵ is CR¹⁰, and X⁶ is CR¹⁰,
(xi) X¹ is C, X² is CH, X³ is N, X⁴ is CR¹⁰, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.
or X⁷ is N, and
(xiii) X¹ is C, X² is NR², X³ is C, X⁴ is CR¹⁰, X⁵ is CR¹⁰, and X⁶ is CR¹⁰.

In some such embodiments, X⁴, X⁵, X⁶, and X⁷ are independently CH or N.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X1 is C, X2 is NR2, X3 is C, X4 is CH, X5 is N, and X6 is CH.

In some embodiments, X⁷ is N, and
(xiii) X¹ is C, X² is NR², X³ is C, X⁴ is CH, X⁵ is CH, and X⁶ is CH.

For example, in these structures, R² can be H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A}. R^{A}, R^{B}, and R^{C} at each occurrence are independently H, alkyl, haloalkyl, or aryl. In some embodiments, R² is methyl. In some embodiments, R² is benzyl (Bn).

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(vi) X¹ is C, X² is O, X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X¹ is C, X² is NR², X³ is C, X⁴ is N, X⁵ is CH, and X⁶ is CH, or
(xii) X¹ is C, X² is NR², X³ is C, X⁴ is CH, X⁵ is N, and X⁶ is CH.

G may or may not contain heteroatoms. In some embodiments, G is a 6-membered ring. In some embodiments, G is a phenyl ring. In some embodiments, G is a pyridine. In some embodiments, G is a pyrimidine. In some embodiments, G is a pyrazine. In some embodiments, G is substituted with one or more alkoxy, such as methoxy. In some embodiments, G is substituted with one or more alkyl, such as methyl. In some embodiments, G is substituted with one or more haloalkyl, such as —CF₃.

In some embodiments, Q⁰ is a metal counter ion, such as sodium, potassium, or lithium.

In some embodiments, Z is halogen. In some embodiments, Z is a group commonly understood as “pseudo halogen”, such as sulfonate, sulfamate, or ester. In some embodiments, Z is bromide, —OSO₂CF₃, —OSO₂Ph, —OSO₂Me, —OSO₂Nme₂, —OC(O)Me, or —OC(O)tBu.

In some embodiments, Q is —BR³R⁴, wherein R³ and R⁴ are independently hydroxy or alkoxy, or R³ and R⁴ together with the boron that they are attached to form a 5-, 6-, or 7-membered ring optionally substituted with one or more alkyl groups. In some particular embodiments, Q is

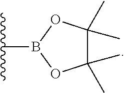 [SMILES: *B1OC(C)(C)C(C)(C)O1]

In some embodiments, M is transition metal. Transition metal includes chemical elements in the d-block of the periodic table (groups 3-12). In some embodiments, M is nickel, palladium, or zirconium.

In some embodiments, R⁷ is cyclohexyl or phenyl.

In some embodiments, n is 2 or 3.

In some embodiments, -M(Y¹)(Y²) is

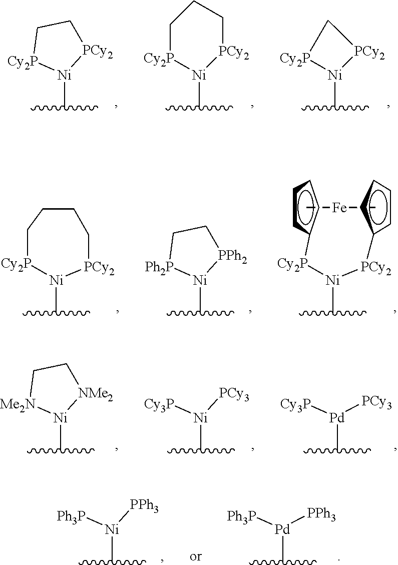

In some embodiments, the compound of formula (III), III(a), or III(a-1) is

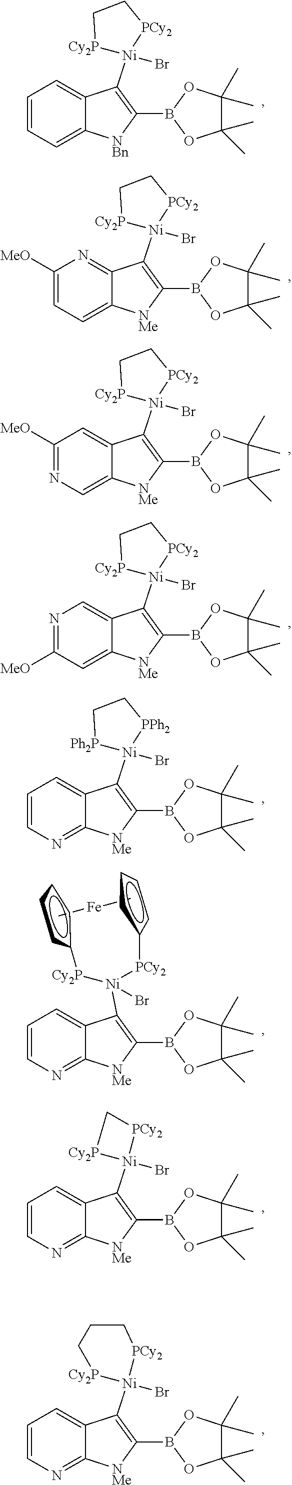

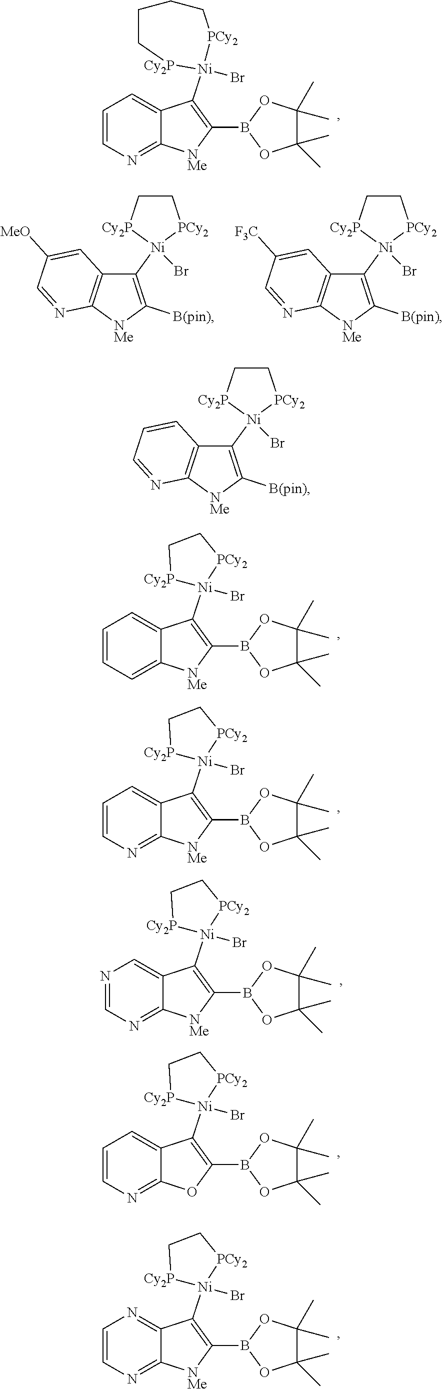

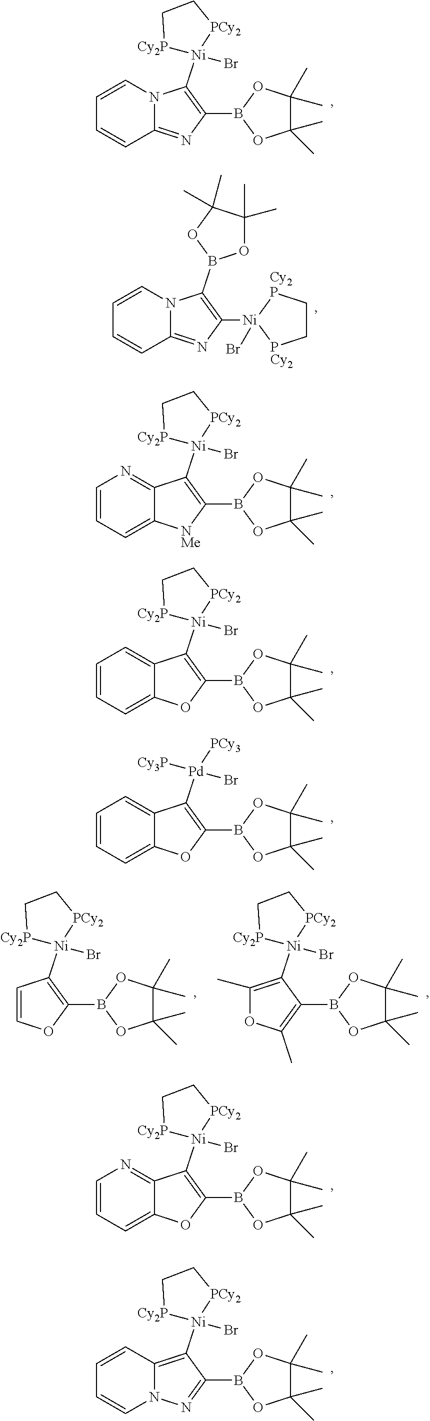

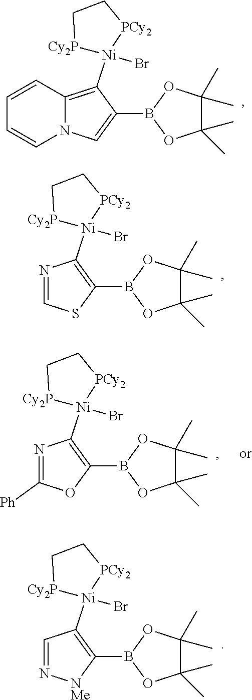

In another aspect, the present disclosure provides a heteroaryne of formula (I), or a salt thereof, which may be produced by the present process. In particular, the present disclosure provides a heteroaryne of formula (I), or a salt thereof:

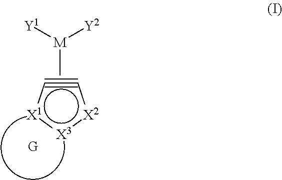

wherein
X¹, X², and X³ are independently C, CR¹, N, NR², S or O;
R¹ and R² at each occurrence are independently H, alkyl, alkoxy, aryl; —C₁₋₄ alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li or —Ag,
   wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

In some embodiments, formula (I) has a structure of formula I(a-1).

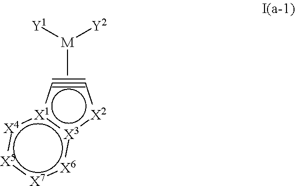

wherein X⁴, X⁵, X⁶, and X⁷ are independently CR¹⁰ or N, and R¹⁰ at each occurrence is independently H, alkyl, halo, alkoxy, or haloalkyl. The other groups are the same as defined herein. For example, X⁴, X⁵, X⁶, and X⁷ are independently CH or N.

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(iii) X¹ is C, X² is O, X³ is C, X⁴ is CR¹⁰ X⁵ is CR¹⁰, and X⁶ is N,
(iv) X¹ is C, X² is NR², X³ is C, X⁴ is N, X⁵ is CR¹⁰, and X⁶ is N,
(v) X¹ is N, X² is N, X³ is C, X⁴ is CR¹⁰, X⁵ is CR¹⁰, and X⁶ is CR¹⁰,
(vi) X¹ is C, X² is O, X³ is C, X⁴ is CR¹⁰, X⁵ is CR¹⁰ and X⁶ is CR¹⁰,
(vii) X¹ is C, X² is NR², X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰,
(ix) X¹ is C, X² is O, X³ is C, X⁴ is N, X⁵ is CR¹⁰, and X⁶ is CR¹⁰,
(x) X¹ is C, X² is N, X³ is N, X⁴ is CR¹⁰, X⁵ is CR¹⁰, and X⁶ is CR¹⁰,
(xi) X¹ is C, X² is CH, X³ is N, X⁴ is CR¹⁰, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.
or X⁷ is N, and
(xiii) X¹ is C, X² is NR², X³ is C, X⁴ is CR¹⁰, X⁵ is CR¹⁰, and X⁶ is CR¹⁰.

In some such embodiments, X⁴, X⁵, X⁶, and X⁷ are independently CH or N.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH.

In some embodiments, X⁷ is N, and
(xiii) X¹ is C, X² is NR², X³ is C, X⁴ is CH, X⁵ is CH, and X⁶ is CH.

For example, in these structures, R² can be H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A}. R^{A}, R^{B}, and R^{C} at each occurrence are independently H, alkyl, haloalkyl, or aryl. In some embodiments, R² is methyl. In some embodiments, R² is benzyl (Bn).

In some embodiments, X⁷ is CR¹⁰, and
(i) X¹ is C, X² is NR², X³ is C, X⁴ is CR¹⁰, X⁵ is CR¹⁰, and X⁶ is N,
(ii) X¹ is C, X² is NR², X³ is C, X⁴ is CR¹⁰, X⁵ is N, and X⁶ is N,
(vi) X¹ is C, X² is O, X³ is C, X⁴ is CR¹⁰, X⁵ is CR¹⁰, and X⁶ is CR¹⁰,
(viii) X¹ is C, X² is NR², X³ is C, X⁴ is N, X⁵ is CR¹⁰, and X⁶ is CR¹⁰, or
(xii) X¹ is C, X² is NR², X³ is C, X⁴ is CR¹⁰, X⁵ is N, and X⁶ is CR¹⁰.

For example, in some embodiments, X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X¹ is C, X² is NR², X³ is C, X⁴ is N, X⁵ is CH, and X⁶ is CH, or
(xii) X¹ is C, X² is NR², X³ is C, X⁴ is CH, X⁵ is N, and X⁶ is CH.

G may or may not contain heteroatoms. In some embodiments, G is a 6-membered ring. In some embodiments, G is a phenyl ring. In some embodiments, G is a pyridine. In some embodiments, G is a pyrimidine. In some embodiments, G is a pyrazine. In some embodiments, G is substituted with one or more alkoxy, such as methoxy. In some embodiments, G is substituted with one or more alkyl, such as methyl. In some embodiments, G is substituted with one or more haloalkyl, such as —CF₃.

In some embodiments, M is transition metal. Transition metal includes chemical elements in the d-block of the periodic table (groups 3-12). In some embodiments, M is nickel, palladium, or zirconium.

In some embodiments, R⁷ is cyclohexyl or phenyl.

In some embodiments, n is 2 or 3.

In some embodiments, -M(Y¹)(Y²) is

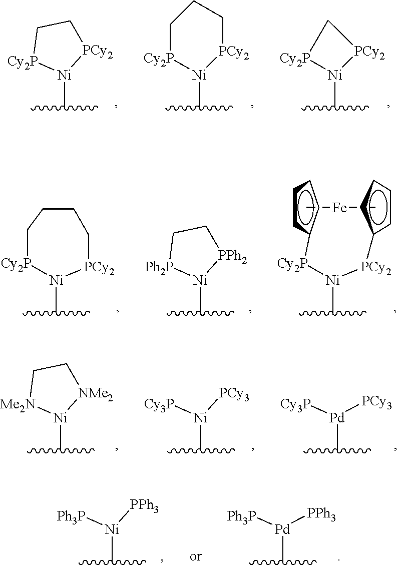

In some embodiments, the heteroaryne of formula (I), I(a), or I(a-1) is

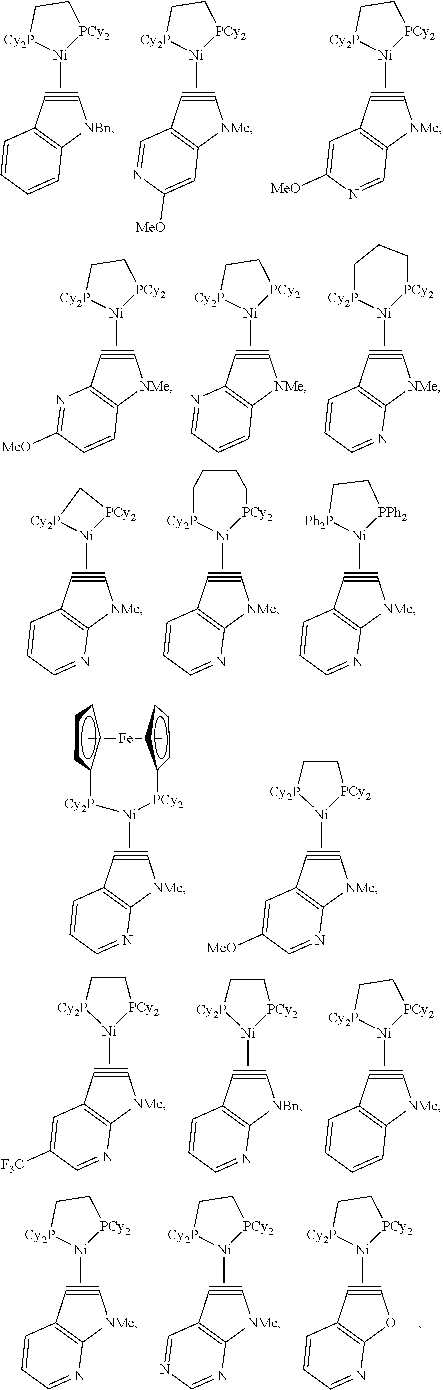

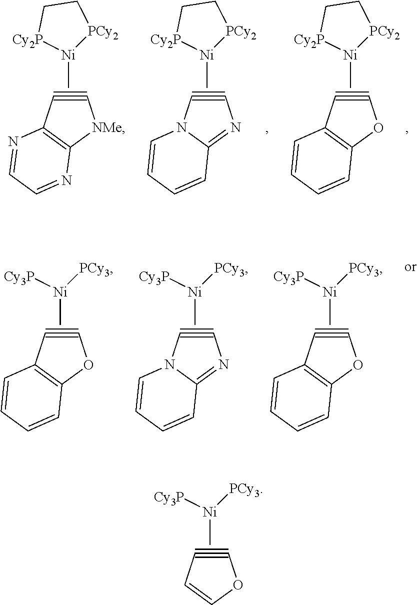

### Synthesis of Molecules of Interest

The compounds disclosed herein and/or prepared according to the methods disclosed herein may serve as synthetic building blocks to build libraries of biologically active molecules with different heterocyclic cores.

In another aspect, the present disclosure provides a method of preparing a target molecule, comprising coupling a compound of formula (I) as described herein with a partner molecule, thereby producing the target molecule.

In some embodiments, the compound of formula (I) as described herein possesses ambiphilic reactivity and may undergo reactions with a variety of partner molecules. Suitable partner molecules include, but are not limited to, nucleophilic, electrophilic, and enophilic coupling partners.

In some embodiments, the coupling reaction is cyclization or nucleophilic addition.

In some embodiments, the partner molecule is

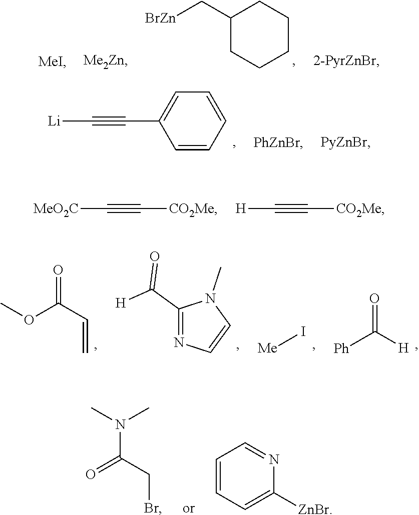

In some embodiments, the target molecule is

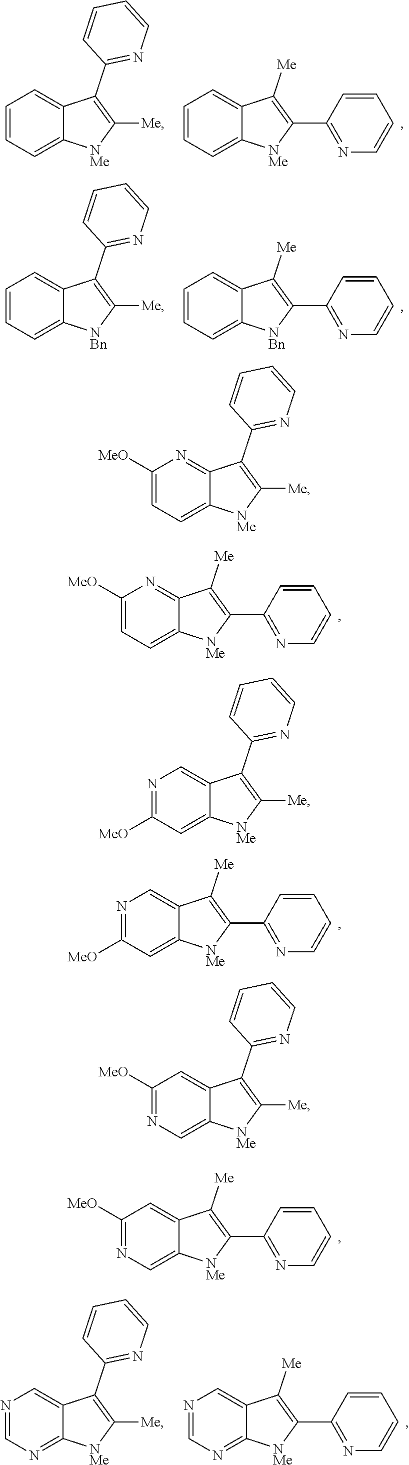

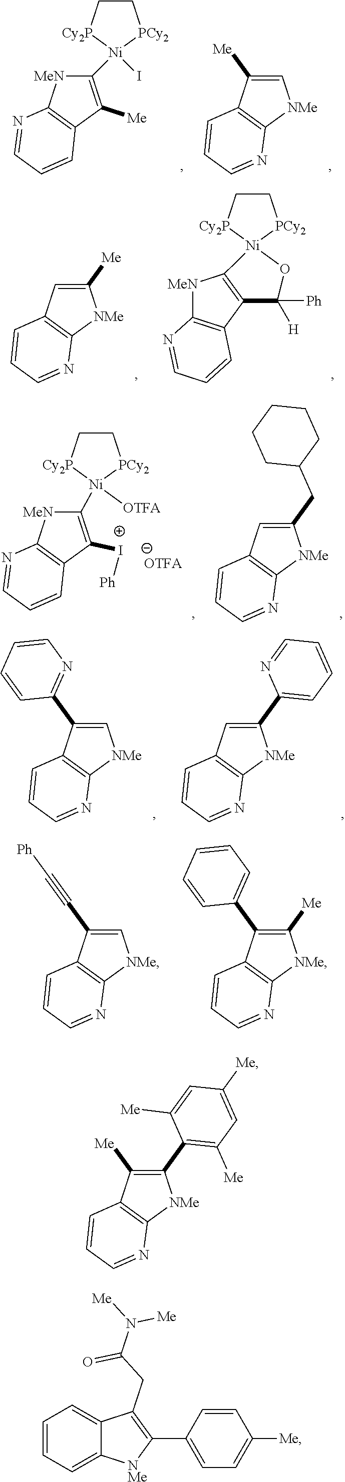

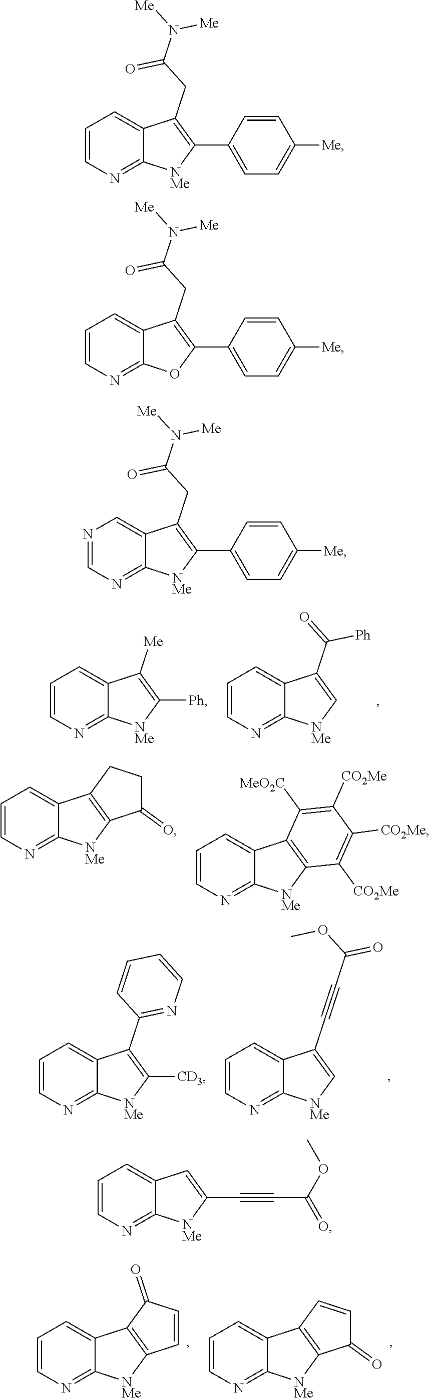

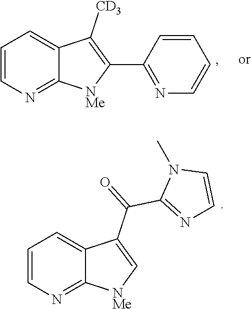 [SMILES: [2H]C([2H])([2H])c1c(-c2ccccn2)n(C)c2ncccc12 ; Cn1ccnc1C(=O)c1cn(C)c2ncccc12]

Other features and advantages of the invention will be apparent from the description of the preferred embodiments thereof, and from the claims. Some aspects and features of the present disclosure are included in the Appendix. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1

Heteroaromatic compounds are essential building blocks for pharmaceuticals, agrochemicals, and materials. N-Heteroarynes, aromatic rings containing a triple bond and an N atom, are attractive synthons because they can undergo difunctionalization in a single step. For example, a variety of six-membered heteroarynes, such as 3,4-pyridyne and 4,5-indolyne, have been used as key intermediates in the synthesis of natural products. Despite its widespread use in the synthesis of natural products, aryne methodology is constrained by the scope of arynes that can be formed.

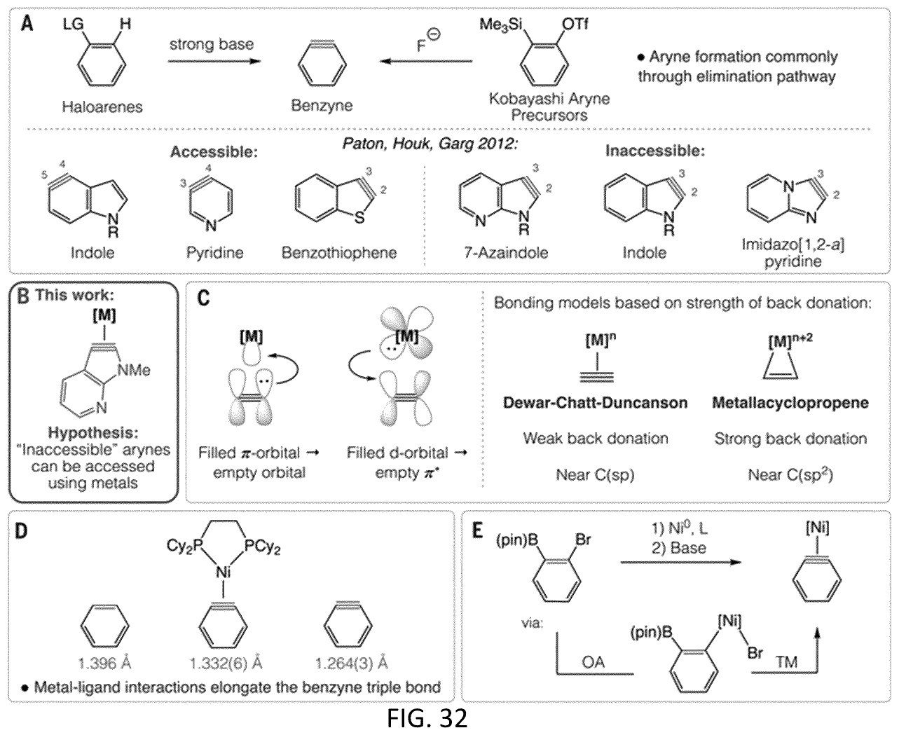

Typical methods to access arynes involve the formation of an anion followed by the expulsion of an appropriate leaving group to generate the triple bond through an elimination reaction (FIG. 32, panel A). Formation of the anion generally requires a harsh base to deprotonate a haloarene for elimination. Alternatively, silylaryl triflate precursors originally developed by Kobayashi's group have been widely used. These aryne precursors can be activated under mild conditions by using a fluoride activator and undergo elimination through a similar mechanism.

A computational model was developed by Paton, Houk, Garg, and colleagues to determine the accessibility of arynes for use in synthesis (FIG. 32, panel A). The model distinguishes accessible from inaccessible arynes by using the heat of dehydrogenation as a metric for alkyne stability. Heteroarynes within six-membered rings, such as 3,4-pyridyne or 4,5-indolyne, were predicted to be accessible. These predictions were confirmed experimentally by Garg and colleagues. Five-membered heteroarynes have been invoked as intermediates for more than 120 years, but their intermediacy has been contested. Based on the computational model, the only five-membered heteroarynes predicted to be accessible contain sulfur, which relieves strain by its size and comparatively long C—S bond length. However, there has been limited success in the formation of free arynes. This computational model also predicts that five-membered N-heteroarynes are inaccessible, meaning that they cannot be formed because of the strain associated with forming a triple bond within a five-membered ring (FIG. 32, panel A). This represents a major limitation of aryne chemistry, as functionalized five-membered N-heterocycles are abundant in pharmaceutically relevant compounds and agrochemicals. Owing to the utility of aryne chemistry and the ubiquity of N-heteroaromatics in drug molecules, agrochemicals, and materials, there remains a need to overcome the limitation and access five-membered N-heterocyclic arynes.

### Hypothesis

Transition metal complexation may alleviate strain in five-membered arynes through metal back-bonding as well as ligand donation (FIG. 32, panel B). When unsaturated C-based ligands bind to metal centers, there are two bonding interactions to consider: i) the donation of electrons from the filled π orbital of the C—C unsaturated bond into an empty metal s or d orbital and ii) the back-donation of a filled metal d orbital to the empty π* orbital of the C—C unsaturated bond (FIG. 32, panel C). Both interactions change the bond order of the unsaturated bond. This phenomenon is well-studied for alkenes and alkynes. Depending on the electronics of the complex, these interactions with an alkyne are described by the Dewar-Chatt-Duncanson model or the metallacyclopropene model (FIG. 32, panel C).

When an aryne is bound to a transition metal, the bond length of the triple bond is elongated through similar bonding interactions to those described above, with the added complexity of the strain induced by the cyclic nature of the aryne and hybridization required to accommodate that. The crystal structure of 1,2-bis(dicyclohexylphosphino)ethane (dcpe) Ni-benzyne shows that the triple bond [1.332(6) Å] is longer than the calculated value in free benzyne [1.264(3) Å] but shorter than a double bond in benzene (1.396 Å) (FIG. 32, panel D). This example demonstrates that the triple bond of an aryne is elongated through metal binding. Wenger and colleagues developed an ortho-borylaryl bromide precursor that can be used to synthesize the Ni-benzyne complex under mild conditions (FIG. 32, panel E). Whereas mechanisms have been proposed that involve aryne precursors that undergo an elimination reaction followed by capture of the aryne by a metal, ortho-borylaryl bromides can be used to definitively generate metal-bound arynes by an inner-sphere oxidative addition followed by intramolecular transmetalation. This distinct mechanism for metal-bound aryne formation opens up the opportunity to address limitations of otherwise inaccessible N-heteroarynes.

### Synthesis

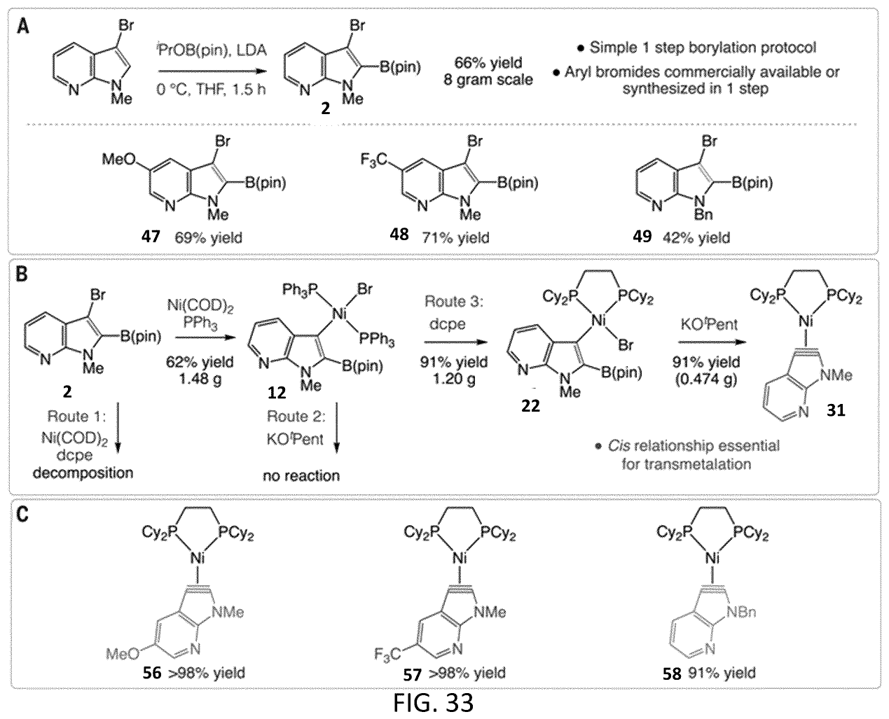

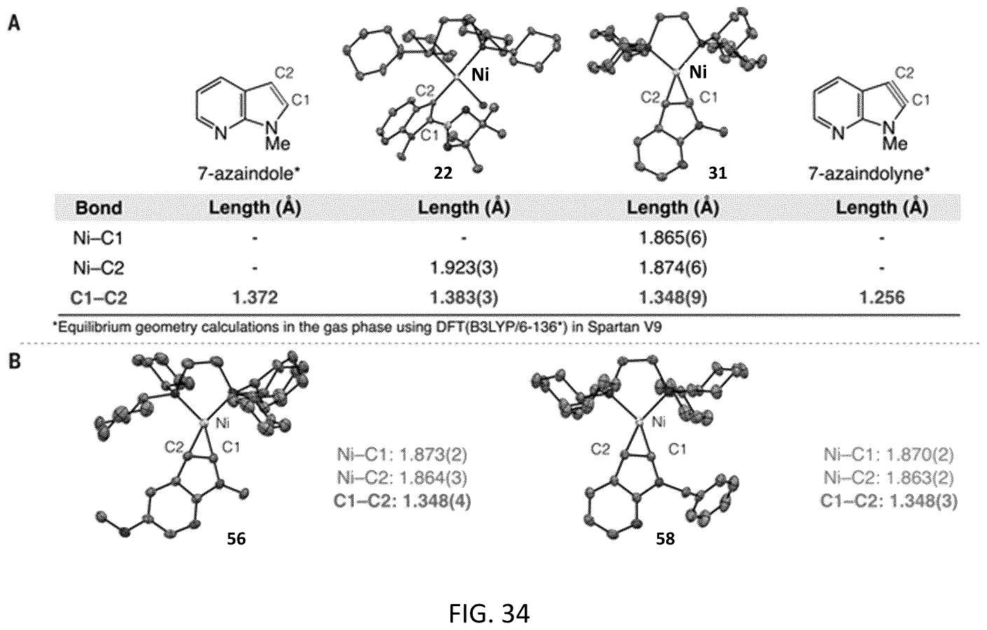

7-azaindole was first targeted because it is the most common azaindole core found in bioactive compounds owing to its hinge H-bonding abilities. A one-pot route to 7-azaindole o-borylaryl bromide precursor 2 was developed from commercially available aryl bromide starting materials (FIG. 33, panel A). This route involves a lithium diisopropylamide (LDA)-promoted borylation with 2-isopropoxy-4,4,5,5-tetramethyl-1,3,2-dioxaborolane [ⁱPrOB(pin)], which avoids the intermediacy of a highly unstable boronic acid (FIG. 33, panel A). Model aryne precursor 2 Was prepared on an 8-g scale with this method. To vary the electronics, 5-Ome- (47) and 5-CF₃-7-azaindole (48) precursors were also synthesized, as was benzyl (Bn)-protected 7-azaindole precursor 49 to study the impact of the N-substituent.

Initial attempts at direct oxidative addition of Ni(COD)₂ with dcpe and 2 did not yield the desired σ-aryl complex 22 (FIG. 33, panel B, route 1). Therefore, the use of a less electron-rich monodentate ligand was necessary when using Ni(COD)₂. Compound 2 underwent oxidative addition with Ni(COD)₂ and two equivalents of PPh₃ to generate an isolable σ-aryl complex 12. The PPh₃ ligands in 12 are in a trans relationship to each other, which prevents transmetalation and results in no reaction upon addition of an activator (route 2). This PPh₃ complex 12 subsequently underwent ligand exchange with a more donating, commercially available bidentate phosphine ligand, dcpe, to generate the desired σ-aryl complex 22 (route 3). The bidentate ligand forces a cis geometry of the arene and bromide ligands. Subsequently, the addition of potassium tert-pentoxide as a soluble activator promoted intramolecular transmetalation, resulting in the formation of the desired Ni-bound 7-azaindolyne complex 31 This complex was characterized by x-ray crystallography, ¹H, ¹³C{¹H}, and ³¹P{¹H} nuclear magnetic resonance (NMR) spectroscopy, and ultraviolet-visible (UV-vis) spectroscopy.

### Crystallographic Characterization

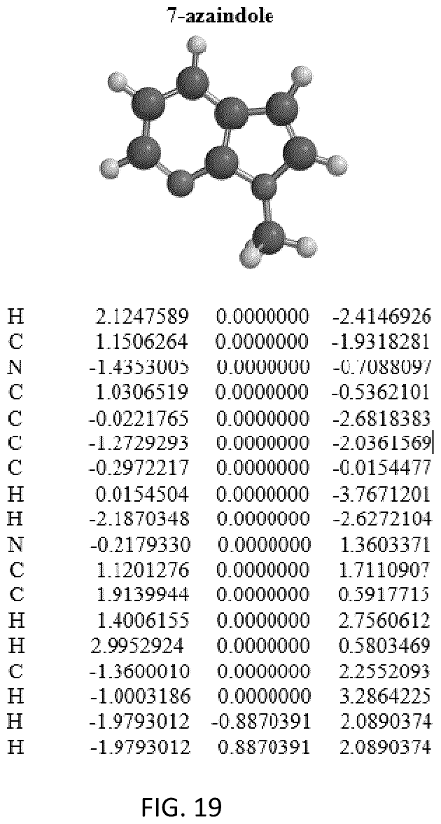

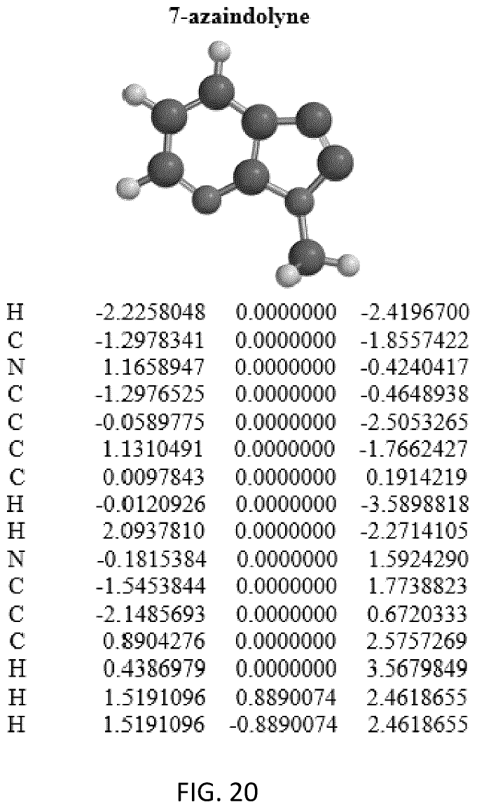

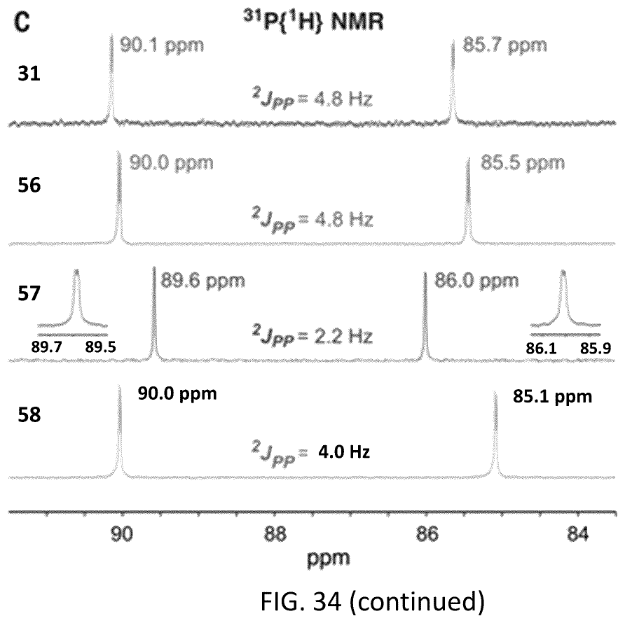

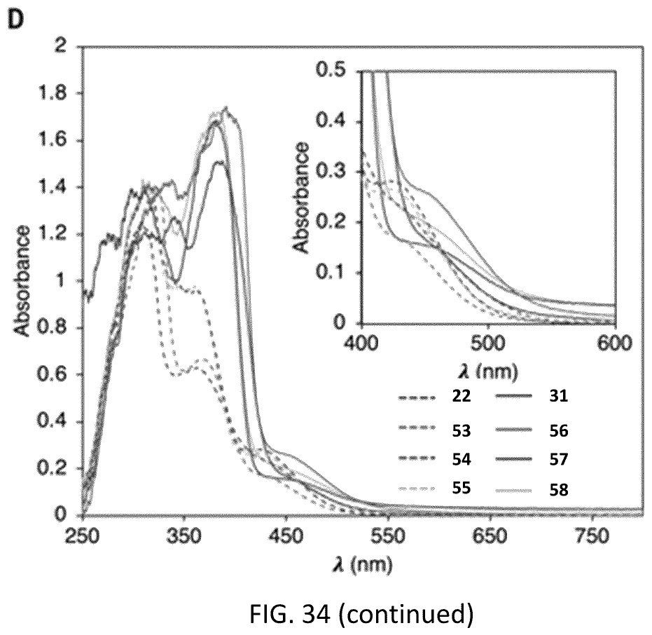

To evaluate the hypothesis that metal-bound five-membered arynes could be formed through strain relief from metal back-bonding, the crystal structures of 22 and 31 were compared with calculated structures of 7-azaindole and 7-azaindolyne (FIG. 34, panel A). Compounds 22 and 31 were crystallized by layering CH₂Cl₂ with pentane and pentane vapor diffusion in a tetrahydrofuran (THF) solution, respectively. The 7-azaindole and 7-azaindolyne structures (FIGS. 19 and 20) were simulated by using equilibrium geometry calculation workflow developed by Paton, Houk, and Garg. The C1 and C2 bond lengths were compared across 7-azaindole, 22, 31, and 7-azaindolyne. As was hypothesized, the C1-C2 aryne bond exhibits elongation in the metal-bound structure 31 [1.348(9) Å] compared with the calculated bond length of the triple bond of 7-azaindolyne (1.256 Å). The C1-C2 bond of 31 is still shorter than the calculated bond length of the double bond in 7-azaindole (1.372 Å) and of the σ-aryl complex 22 [1.383(3) Å]. This demonstrates the aryne character of 31. The C1-C2 bond of 31 is also elongated compared with the previously reported dpce-Ni benzothiophyne structure [average of 1.332(7) Å], indicating that more back-donation is necessary to form the smaller arynes. The distance from the Ni center to the C1 terminus of the aryne bond of 31 [1.865(6) Å] is shorter than the Ni—C2 distance [1.874(6) Å] by 0.009 Å, demonstrating a slight difference in donation of the ancillary ligands and back-donation to the two aryne carbons in 31.

The C1-C2 aryne bond length is the same across structures 31, 56, and 58 (FIG. 34, panel B), which vary electronically and sterically. However, the Ni—C distances vary. In 31, the longer Ni—C distance is Ni—C2, whereas in 56 and 58, the longer Ni—C distance is Ni—C1. Multiple attempts to crystallize 57 failed to produce sufficiently high-quality single crystals. However, the difference in electronics across the whole series of 31, 56, 57, and 58 can be observed in the ³¹P{¹H} NMR spectra.

### Spectroscopic Characterization

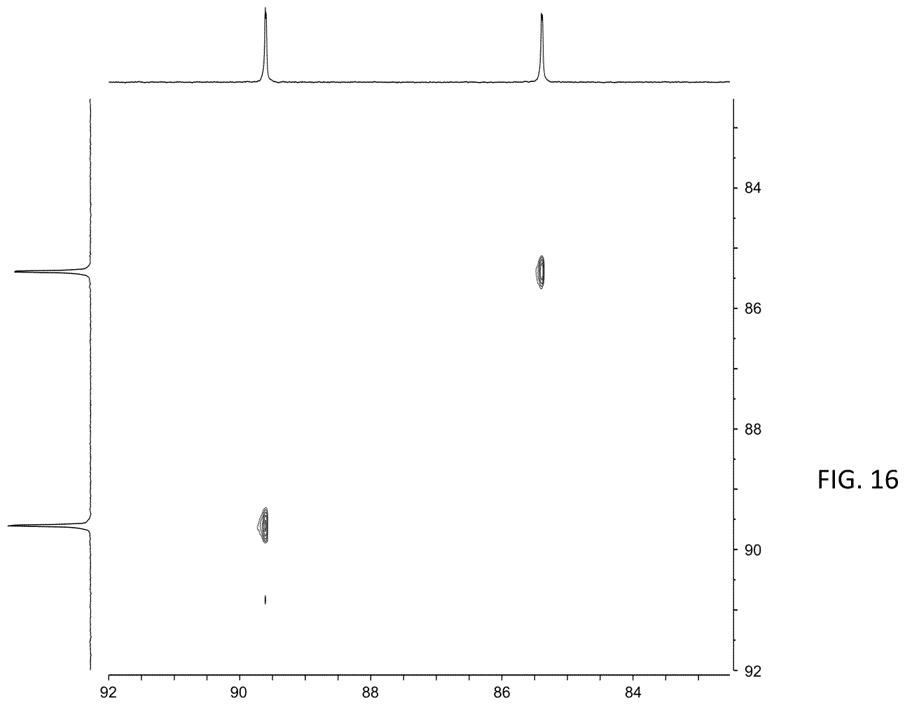

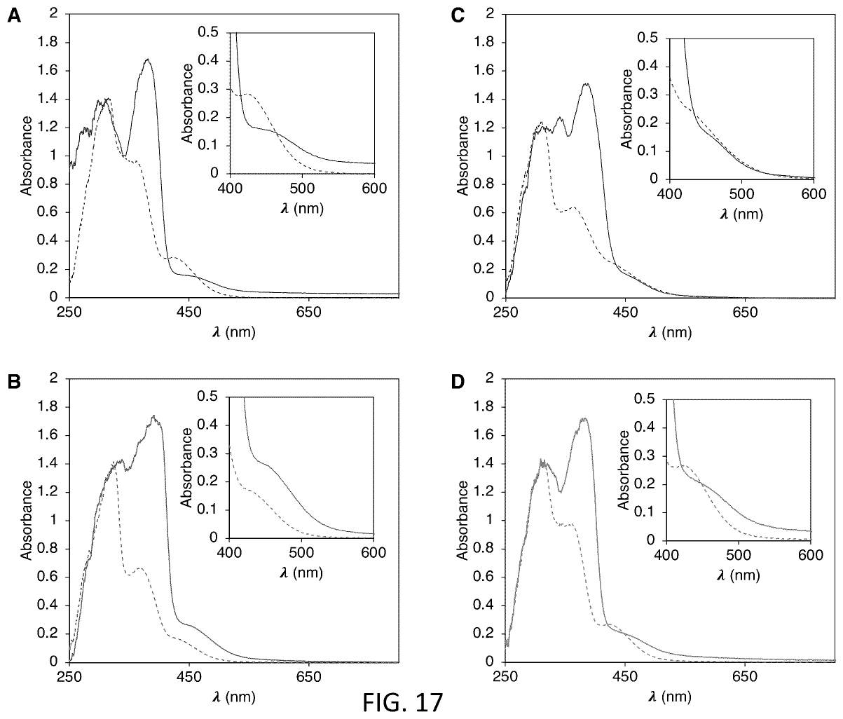

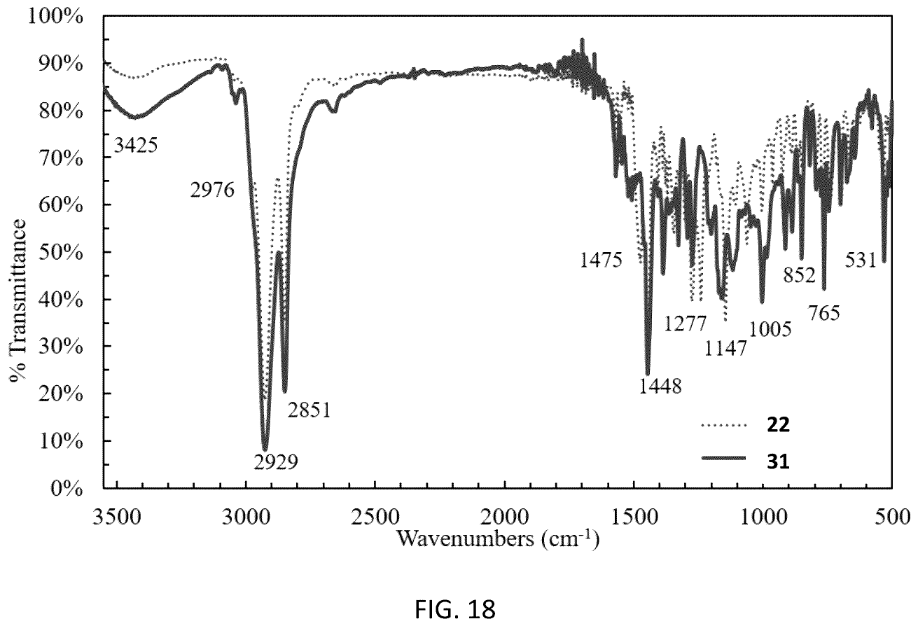

The chemical shift and coupling constants in ³¹P{¹H} NMR spectroscopy can be an indication of donation by the phosphine ligands. Regarding chemical shift, complexes 31 to 56-58 [peaks in the 90 to 85 parts per million (ppm) range] are far more deshielded than the dcpe-Ni complex of benzyne, which has a chemical shift of 78.4 ppm (FIG. 34, panel C). The most downfield ³¹P shift (90.1 to 89.6 ppm, P trans to C1) only changes by 0.5 ppm across all four arynes 31 to 56-58, and the upfield peak (86.0 to 85.1 ppm, P trans to C2) manifests a nearly 1.0-ppm shift difference, indicating a stronger change in the donation of the upfield P donor. Both signals are downfield from those reported for the dcpe Ni-benzothiophyne, which appear at 87.7 and 83.5 ppm, indicating the increased phosphine donation to the Ni and resulting back-donation to the aryne in 31 to 56-58. The ²J_{PP} coupling constant is an indication of σ donation of a phosphine to the Ni center. Notably, the ²J_{PP} coupling constants show very little variation (4.0 to 4.8 Hz range) across 31, 56, and 58. By contrast, the CF₃-substituted aryne 57 has a ²J_{PP} coupling constant of 2.2 Hz, indicating a large difference in electronic structure compared with 31, 56, and 58. This smaller coupling constant is attributable to the electron-deficient CF₃ aryne withdrawing more electron density from the phosphine donors. The ²J_{PP} coupling constants (average of 7.1 Hz) of the dcpe Ni-benzothiophyne complex are much larger, indicating a difference in the phosphine a donation into a sulfur versus nitrogen heteroaryne, the latter of which requires more strain relief. This observation is in agreement with the crystallographic analysis vide supra. In contrast to a recently reported Ni-bound six-membered aryne, the room temperature two-dimensional (2D)³¹P{¹H}—³¹P{¹H} nuclear Overhauser effect spectroscopy exchange spectrum indicates that 31 does not undergo aryne dissociation or exchange in a mixing period of 3 s (FIG. 16).

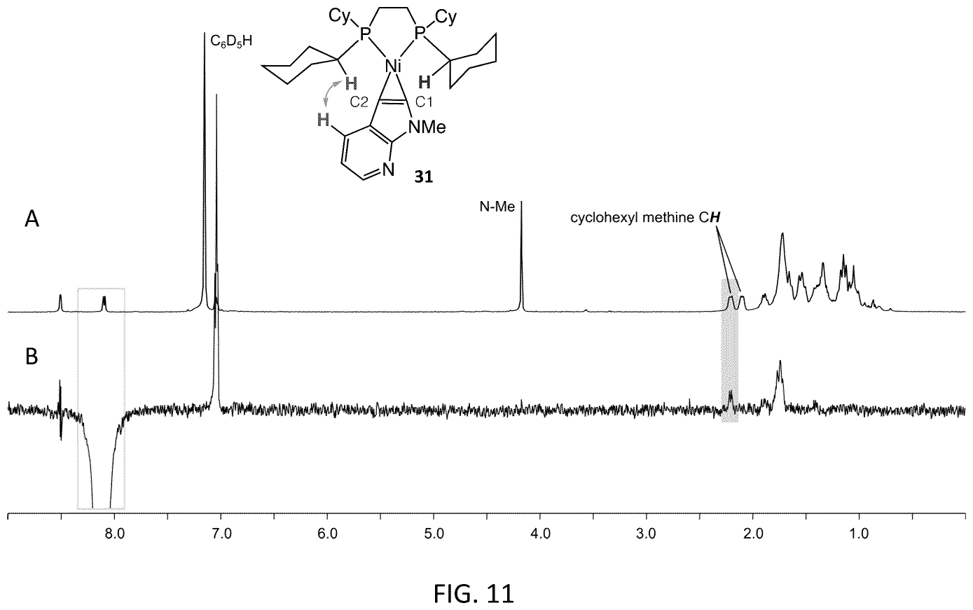

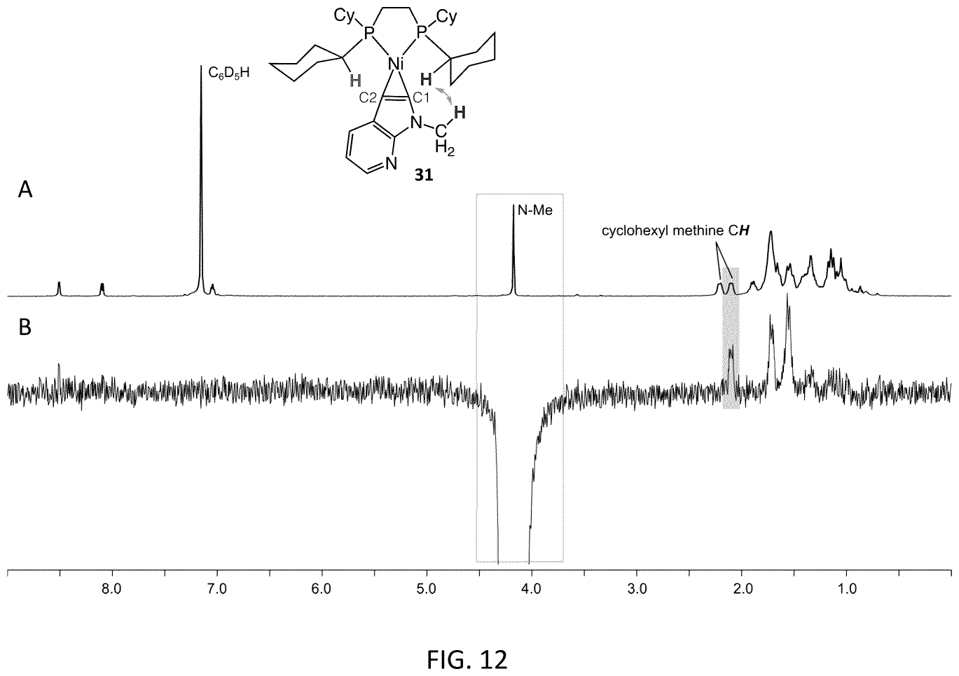

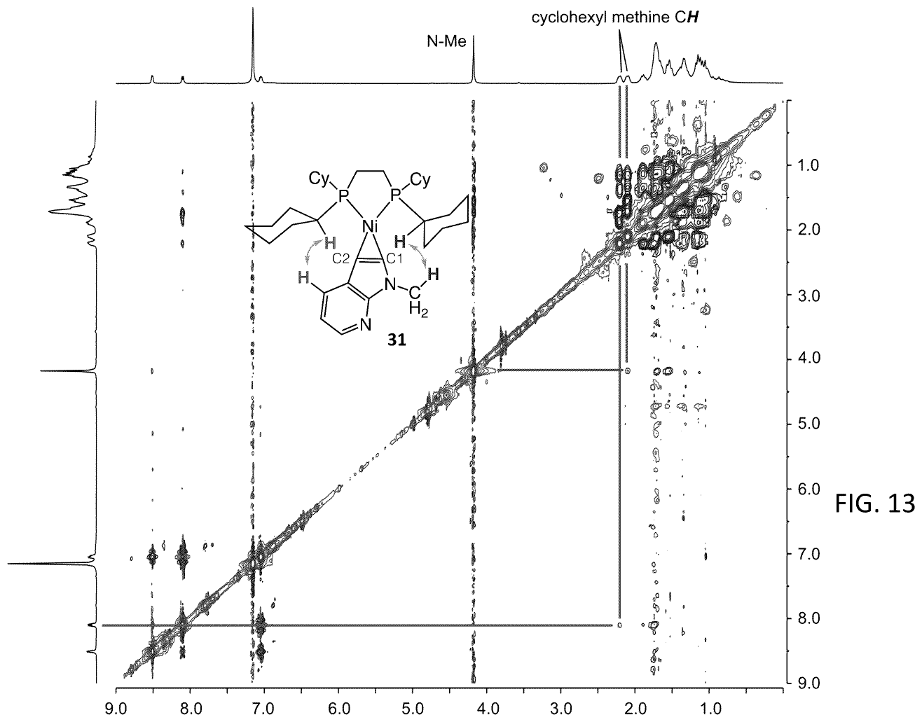

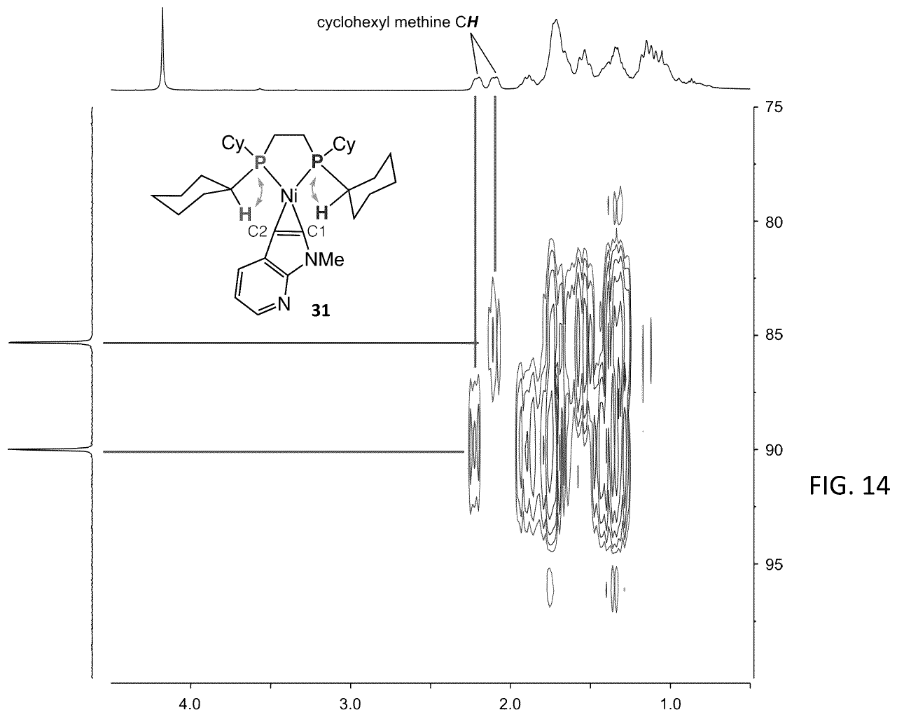

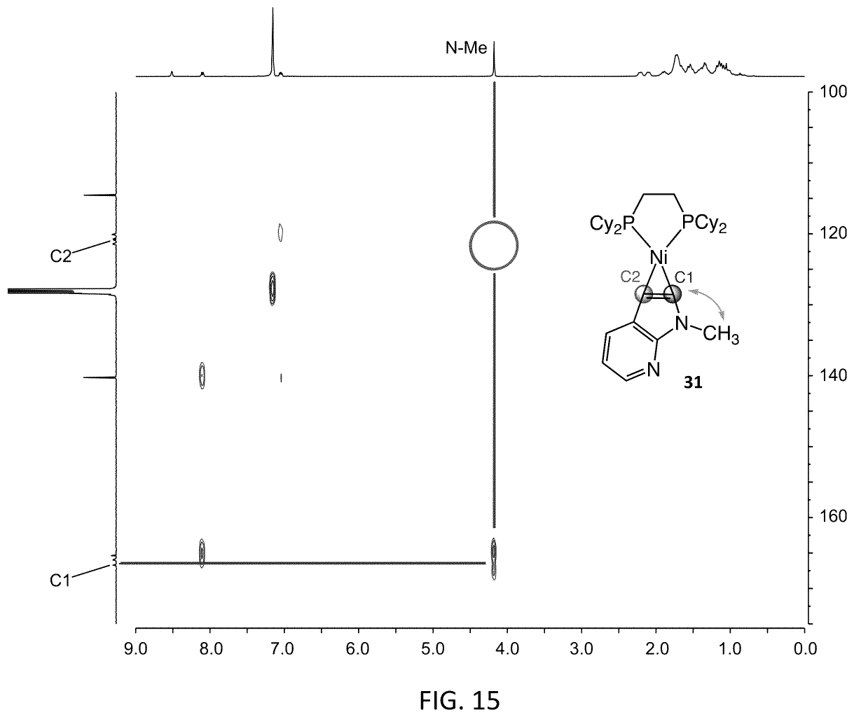

The ¹³C{¹H} NMR chemical shifts of the two aryne carbons also indicate how electronically distinct these five-membered N-heterocyclic arynes are from other known arynes. The C1 and C2 signals are doublets of doublets owing to coupling to both chemically inequivalent P donors. Across the series of four complexes 31 to 56-58, C1 has a resonance range of 170.6 to 166.0 ppm, and C2 ranges from 120.7 to 119.9 ppm, as confirmed by 2D NMR spectroscopy (FIG. 11 to FIG. 15). This contrasts with the ¹³C{¹H} NMR chemical shift reported for dcpe Ni-benzyne, which appears at 146.0 ppm, and the dcpe Ni-benzothiophyne complex, where C1 and C2 have chemical shifts of 154.9 and 152.6 ppm, respectively.

Fairly little is known about how the electronic structure of metal-aryne complexes manifests in UV-vis spectroscopy. Therefore, the spectra of the σ-aryl complexes 22 to 53-55 were compared with the aryne complexes 31 to 56-58 (FIG. 34, panel D). Ligand-based π-π transitions (250 to 350 nm) were observed in all the spectra. The peaks at 400 to 500 nm with ε≈700 to 1300 M⁻¹ cm⁻¹ redshift slightly in the arynes compared with those of the σ-aryl complexes. The most distinguishing feature of the aryne spectra of 31 to 56-58 is a strong band with maximum wavelength range of 385 to 400 nm absent for 22 to 53-55.

### Transmetallation Studies

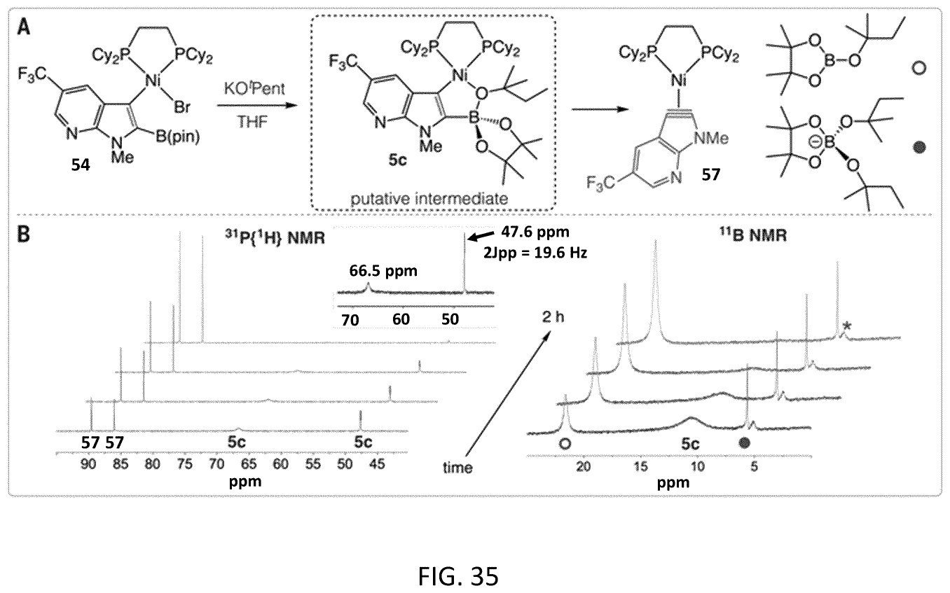

The transformation of 54 to 57 was slow enough to observe an alkoxide-activated intermediate 5c prior to transmetalation (FIG. 35, panel A). The reaction was monitored by ³¹P{¹H} and ¹¹B NMR spectroscopy over a 2-hour period (FIG. 35, panel B). The ³¹P{¹H} NMR spectrum showed a small amount of aryne formation within the time it took to collect the initial spectrum, approximately 10 min. Two additional resonances were observed: a broad peak at 66.5 ppm and a sharp doublet at 47.6 ppm with a ²J_{PP} value of 19.6 Hz. Over time, these two additional resonances, which maintained a 1:1 ratio, disappeared, and the aryne signals grew in. The ¹¹B NMR also showed the ^{t}PentO-B(pin) product of transmetalation as well as the [(^{t}PentO)₂—B(pin)]⁻ borate by-product at the first time point. An additional broad resonance was also observed in the borate region at 10.4 ppm. This shift and broad peak are consistent with a structure that Thomas, Denmark, and colleagues were able to observe of an activated borate prior to transmetalation. As with the ³¹P{¹H} NMR spectra, this broad resonance at 10.4 ppm in the ¹¹B NMR spectra disappears over time in proportion to growth of the by-product of transmetalation. With this evidence and precedent from Thomas, Denmark, and colleagues, we proposed the activated borate species with a bound alkoxide 5c. The Ni-bound alkoxide could alternatively be one portion of the pinacolate, leading to a mixture of structures and a broad ³¹P{¹H} resonance for the phosphine trans to that position. Regardless of which oxygen is bound, the NMR spectra indicate an activated pretransmetalation complex at room temperature.

### Reactivity

Late transition metal-bound arynes are generally regarded as nucleophilic in contrast to their non-metal-bound counterparts, which are electrophilic. As demonstrated above, there is far more elongation of the aryne bond in the x-ray analysis of 31 compared with that of other Ni-bound arynes, and the ³¹P{¹H} NMR spectroscopic analysis demonstrates remarkable differences as well. These structural and electronic characteristics could lead to late transition metal aryne reactivity, which is underexplored. Additionally, there could be inherent regioselectivity based on the electronic difference between the two termini of the aryne bond, as evidenced by the ¹³C{¹H} NMR spectra. Indeed, these arynes demonstrate notable reactivity as ambiphiles, reacting not only with electrophiles but also with nucleophiles. The ambiphilic reactivity of this functional group for five-membered N-heterocycles provides a versatile common intermediate that can couple with a wide variety of partners. The ligand can be designed to stabilize the aryne or enhance reactivity of the aryne.

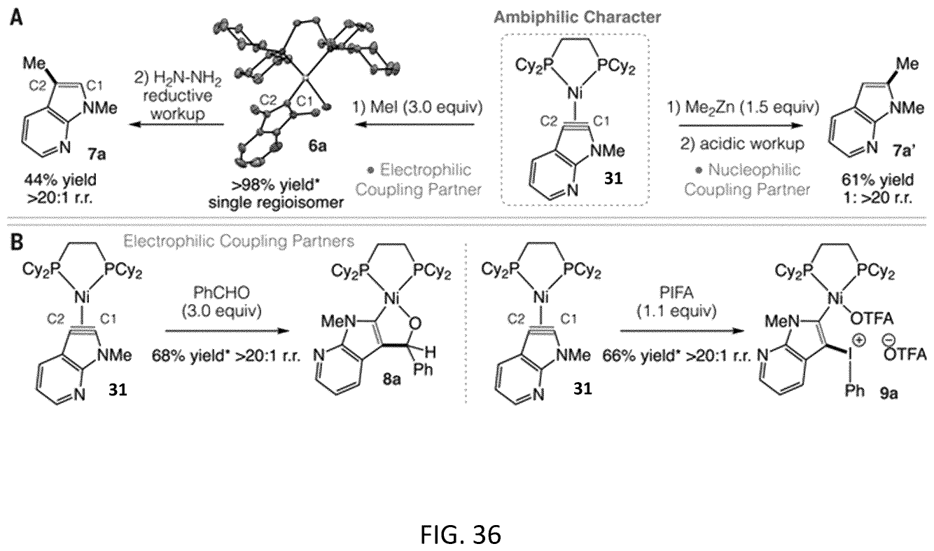

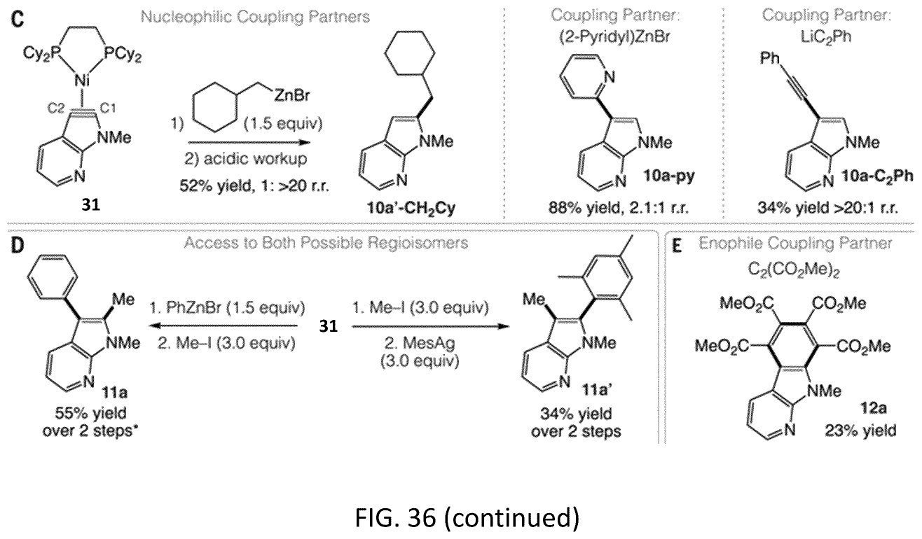

Complex 31 was found to exhibit ambiphilic reactivity in the presence of either electrophilic or nucleophilic methylation reagents. In alignment with known nickel-aryne reactivity, methyl iodide reacts with aryne complex 31 to generate monofunctionalized σ-aryl complex 6a in >98% conversion as a single regioisomer (FIG. 36, panel A). Crystallographic analysis confirmed functionalization at the C2 position. Reaction preferentially at this position aligns with the Ni—C2 distance being longer in keeping with a weaker interaction than the Ni—C1 contact (FIG. 34, panel A). This distinct regiocontrol is not observed in six-membered arynes with C2-symmetric phosphine ligand environments. Reductive workup with hydrazine released product 7a. Complex 31 also reacts with the nucleophile Me₂Zn; an acidic workup to quench excess Me₂Zn generated 7a′ in 61% yield and 1:>20 regioisomeric ratio (r.r.) with reaction at the C1 position of the azaindole favored (FIG. 36, panel A). This notable reactivity with nucleophiles maintains the electrophilic character that is prototypical of non-metal-bound arynes but is understudied in metal-bound arynes. This result also illustrates that there is potential to preferentially obtain either regioisomer depending on the philicity of the coupling partner.

Other classes of electrophiles were also explored (FIG. 36, panel B). Carbonyls react to generate metal-alkoxide complex 8a in 68% yield and >20:1 r.r. (Bis(trifluoroacetoxy)iodo)benzene (PIFA) is known to oxidize low-valent Ni as well as provide an electrophilic source of trifluoroacetoxy groups. We hypothesized that upon exposure to PIFA, aryne complex 31 would undergo trifluoroacetoxylation. However, upon exposure to PIFA, complex 31 generated 7-azaindoleiodonium Ni complex 9a in 66% isolated yield. Complex 9a was characterized crystallographically. This reaction outcome is likely an indication that the back-donation into the aryne bond restricts the Ni center from undergoing facile oxidation.

Markedly for a Ni-bound aryne, complex 31 reacts with the nucleophile (CyCH₂)ZnBr to generate (after an acidic workup) 10a′—CH₂Cy in 52% yield and as a single regioisomer with the reaction at the C1 position of the azaindole favored in a 1:>20 r.r. (FIG. 36, panel C). Whereas alkyl organometallic coupling partners preferentially reacted at the C1 position, C(sp²) coupling partner (2-pyridyl)ZnBr formed 10a-py upon acidic workup in 86% yield and 2.1:1 r.r. favoring the C2 substituted position. Following this same hybridization trend, Li phenylacetylide can be used to install an alkynyl group in 34% yield with a >20:1 r.r. favoring substitution at C2.

Following each of these monofunctionalizations, a σ-aryl complex (such as 6a, 8a, and 9a) is formed and can react further to generate difunctionalized products. These σ-aryl complexes have been widely studied as intermediates in cross-coupling, Buchwald Hartwig amination, halogenation reactions, and many others. There have been notable reports of the utility of stoichiometric organometallic metal complexes for applications in drug development. Therefore, one-pot multicomponent difunctionalizations were undertaken (FIG. 36, panel D). Upon the addition of PhZnBr, complex 31 underwent insertion to give a putative σ-aryl intermediate, which allowed for a second functionalization upon the addition of MeI to give 11a in 55% isolated yield of a single regioisomer. It is likely that the r.r. is enhanced in the isolated difunctionalized product compared with that of the monofunctionaled product through both the preferential reaction of one regioisomer of the intermediate σ-aryl complex and chromatography during separation. Because complex 31 is ambiphilic, the reverse addition of the coupling partners of MeI to form complex 6a followed by redox-active transmetalation organometallic reagent MesAg generated the opposite regioisomer of 11a′ exclusively in 34% yield over two steps, which further highlights the potential to expand chemical space from a single starting point.

Reactivity of complex 31 was explored with prototypical aryne enophile dimethyl acetylenedicarboxylate (DMAD) to generate trace amounts of annulated product 12a. The side-product of this reaction is trimerized DMAD, which forms when the dpce-Ni(0) complex is released following reaction of the aryne, leading to low yields. To control the formation of a Ni(0) intermediate, which leads to this side-product, an oxidant can be used to increase the yield slightly to generate 12a in 23% yield (FIG. 36, panel E).

More examples on functionalization of the aryne complexes are disclosed in Example 4.

### CONCLUSION

Five-membered 7-azaindolyne can be accessed through relieving ring strain from binding to Ni. Computational collaborations can help to fully understand and expand the scope of now-accessible N-heterocyclic arynes following the path of Paton, Houk, and Garg. This would provide a universal synthon that can be derivatized to build libraries of potentially biologically active molecules with different heterocyclic cores. New reactions involving five-membered N-heteroarynes can now be developed with additional heteroatom and carbon coupling partners. For example, regioselectivity in metal-bound aryne functionalizations can be enhanced, and five-membered aryne transformations can be carried out in catalytic reactions (e.g., using o-borylaryl pseudohalide aryne precursors). These studies will provide the synthetic community a new platform for synthesizing decorated heterocycles.

### Example 2 Modification of the Reaction Scope

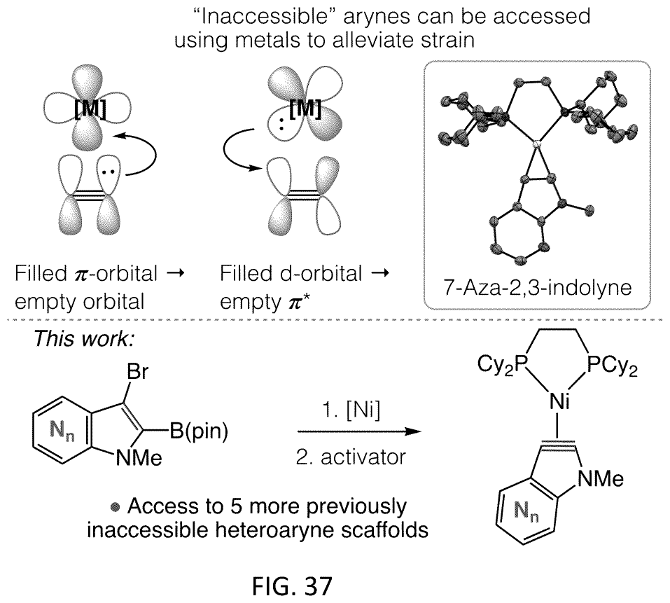

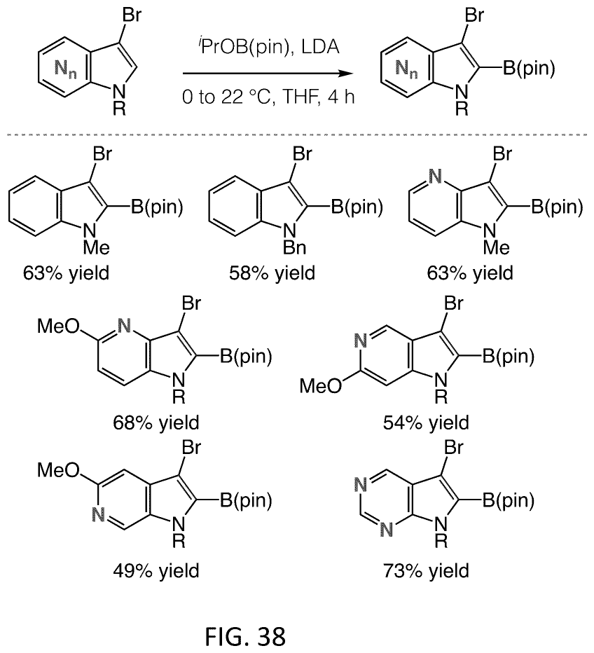

5-membered inaccessible arynes, such as Ni-bound 7-aza-2,3-indolyne, can be accessed using a metal to relieve the strain associated with a triple bond in a 5-membered ring through metal-ligand interactions such as σ-donation and π-back donation, as disclosed in Example 1. A crystal structure revealed that Ni-bound 7-aza-2,3-indolyne had a significantly elongated aryne bond compared to the calculated value but was still shorter than a typical arene double bond, demonstrating that a large amount of π-back donation is necessary to access 5-membered N-heterocyclic arynes. This complex displayed remarkable ambiphilic reactivity as compared to the typical nucleophilic character of Ni-bound benzyne, further demonstrating the unique electronic structure of 5-membered N-heteroarynes (FIG. 37).

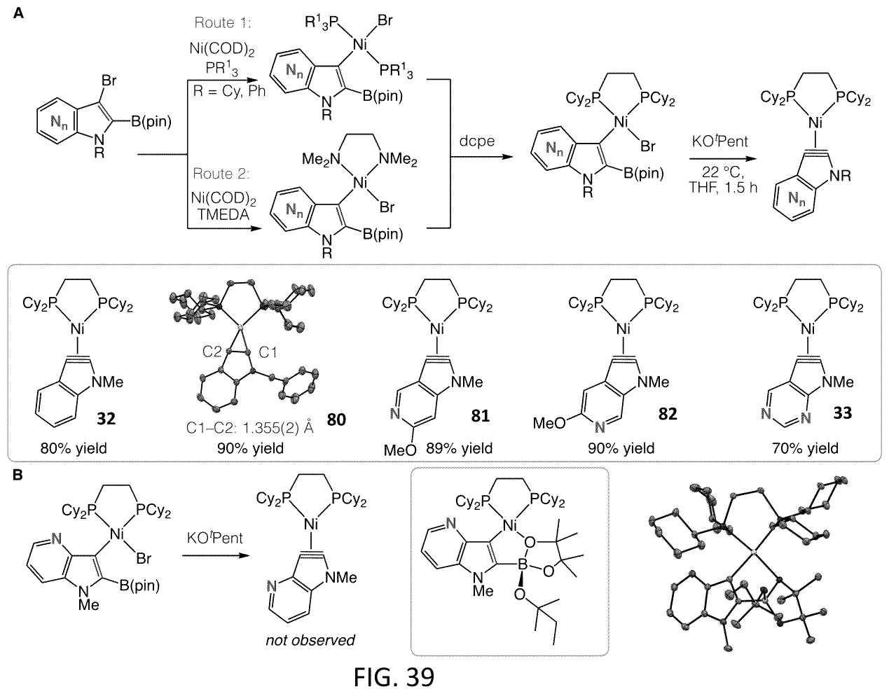

The newly accessed 7-azaindolyne can be extended to other classes of 5-membered N-heterocyclic arynes (FIG. 37). This would help to make 5-membered N-heteroarynes a more universal synthon, aiding in the synthesis of libraries of biologically active molecules. Disclosed herein are 5 new classes of N-heteroarynes that have never been accessed before (FIG. 37). Aryne formation occurs at different rate and efficiencies depending on the heterocycle identity (FIG. 39). The electronics of the aryne clearly play an important role as electronically activating groups can be used to tune reactivity. Solid state structural studies reveal that the aryne bond length changes with heterocycle identity as well.

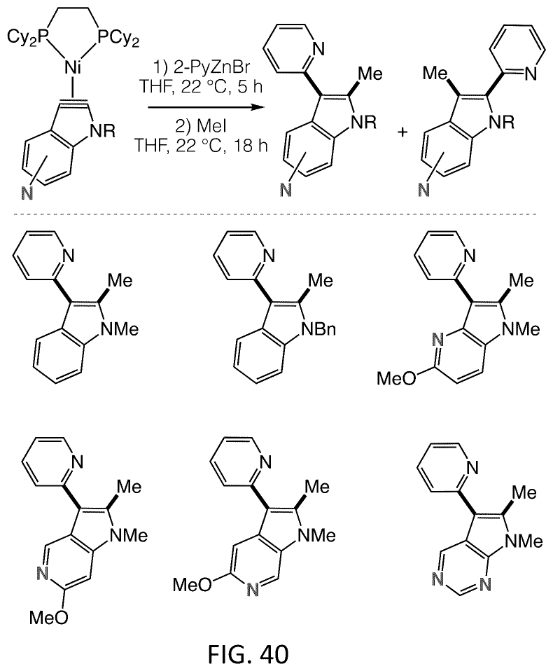

Additionally, functionalization of the 5-membered N-heterocyclic arynes disclosed herein can be carried out, giving rise to a variety of di-substituted heterocyclic products (FIG. 40 and Example 4). The functionalization reactions also reveal differences in aryne structure depending on the heterocycle as different regioselectivities are observed.

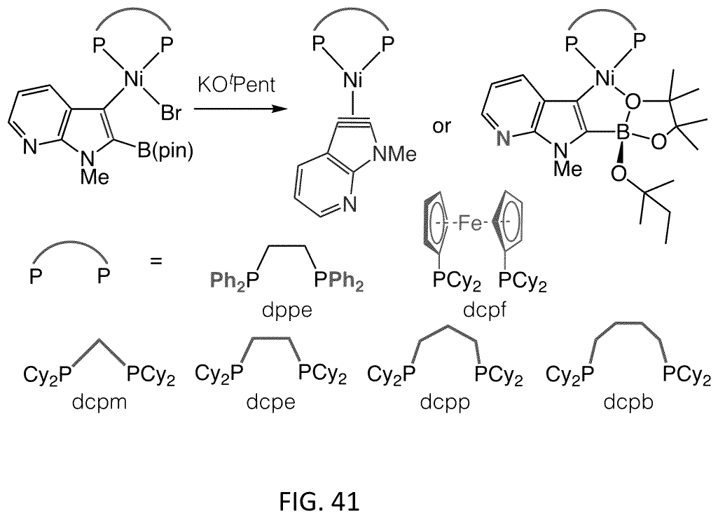

During functionalization of the 7-azaindolyne, it was observed that most coupling partners (e.g., C(sp³) nucleophiles and electrophiles, C(sp) nucleophiles, aldehydes, iodoniums) gave rise to excellent regioselectivity. This is in alignment with the Ni—C1 and Ni—C2 bond lengths being different meaning one site is electronically differentiated from the other. However, with C(sp²) nucleophiles such as 2-PyZnBr, poor regioselectivity was observed. It was hypothesized that changing the identity of the bidentate phosphine ligand could impact both reactivity and selectivity. Reactivity could be impacted by the donor ability of the phosphine ligand which is controlled by the linker length and linker identity, and thus geometry and orbital overlap of the metal and phosphine ligand which consequently impacts the amount of back donation into the aryne bond. Additionally, the non-linker groups on the phosphine impact the amount of donation the phosphine can give which in turn also effects the amount of electron density on the metal center which can be back donated to the aryne. Regioselective addition could also be impacted by these two phenomenon by changing the Ni—C bond lengths. Various bidentate ligands are disclosed herein (FIG. 41).

### Example 3 Synthesis and Characterization

Unless specified, all chemical transformations were performed in a glovebox and using standard Schlenk line techniques. Dimethyl acetylenedicarboxylate (DMAD) and potassium persulfate (K₂S₂O₈) was purchased from Thermo Fischer Scientific and used as received. Hydrazine (H₂NNH₂) was purchased from Acros Organics and stored in the fridge. N-bromosuccinamide (NBS) was purchased from Alfa Aeser and stored in the freezer. Sodium hydride (NaH) (90% emulsion in celite) was purchased from Aldrich and stored in a dessicator. Dibromomethane (DBM) was purchased from Oakwood Chemical and used as I. Lithium diisoproylamide (LDA) solution (1 M in THF), 2-isopropoxy-4,4,5,5-tetramethyl-1,3,2-dioxaborolane (ⁱPrOB(pin)), dimethyl zinc (Me₂Zn) (1 M in heptane), benzaldehyde (PhCHO), (bis(trifluoroacetoxy)iodo)benzene (PIFA), (cyclohexylmethyl)zinc bromide solution (0.5 M in THF), phenylzinc bromide (PhZnBr) solution (0.5 M in THF), 2-pyridylzinc bromide solution (0.5 M in THF), lithium phenylacetylide solution (1 M in THF), and mesitylene were purchased from Sigma-Aldrich and used as received. Ni(cod)₂, and dicyclohexylphosphinoethane (dcpe) and potassium tert-butoxide (KO^{t}Bu) were purchased from Strem and used as received. Triphenylphoshine (PPh₃) and 2-Isopropoxy-4,4,5,5-tetramethyl-1,3,2-dioxaborolane (ⁱPrOB(pin)) were purchased from Sigma-Aldrich and used as received. Potassium tert-pentoxide (Kotpent) solution was purchased from Sigma-Aldrich. To prevent decomposition and preserve the accuracy of the potassium tert-pentoxide solution concentration, in a glovebox under N₂ atmosphere, 5 mL aliquots were taken from the bottle at a time and stored in a glass vial sealed with a Teflon-lined cap in the glovebox for up to 2 months. Aliquots to be used for aryne forming reactions were taken from the glass vial. After 2 months, the tert-pentoxide solution in the vial began to discolor and results in aryne reactions became unreproducible. Substrate precursors (3-Bromo-1-methyl-1H-pyrrolo[2,3-b]pyridine, 3-Bromo-4,7-diazaindole, 3-Bromo-1H-pyrrolo[3,2-b]pyridine, 5-Bromo-7-methyl-71-pyrrolo[2,3-d]pyrimidine, 3-Bromo-1H-1-pyrrolo[3,2-c]pyridine, 2-Bromoimidazo[1,2-a]pyridine) were purchased from Ambeed. Diethyl ether, tetrahydrofuran, toluene, dichloromethane, and pentane were dried on a Pure Process Technology solvent purification system and stored over activated 4 Å molecular sieves under an N₂ atmosphere. Anhydrous N,N-dimethylformamide (DMF) was purchased from Sigma-Aldrich. Celite and molecular sieves were dried by heating in an oven at 120° C. for 18 hours then at 200° C. under reduced pressure for 48-72 hours. (NOTE: The aryne complexes are very sensitive to the presence of water. Vigorous drying of all solvents, filter materials, and coupling patterns are vital to preventing decomposition.) 2-bromo-dimethyl acetamide was purchased from Ambeed and stored over activated 4 Å molecular sieves. 4-methylphenylboronic acid was purchased from TCI (Tokyo Chemical Industry Co. Ltd) and used as received.

Isolated yields of compounds were obtained on a Teledyne ISCO CombiFlash NextGen 300+ instrument using gold high performance columns. Isolations by preparatory TLC were performed using a Miles Scientific UNIPLATE 20×20 cm 1000 micron preparatory TLC plate.

¹H, ¹³C and ¹⁹F NMR spectra were recorded on Bruker AVANCE 400 MHz and 500 MHx spectrometers. Chemical shifts are referenced with respect to residual protiosolvents: 7.26 ppm CD₂HCl₃, 7.16 (C₆D₅H), and 5.32 (CDHCl₂) for ¹H NMR; 77.16 ppm (CDCl₃), 128.06 (C₆D₆), and 53.84 (CD₂Cl₂) for ¹³C.

IR spectra of compounds were collected using a Bruker model Alpha II IR spectrometer, with sample prepared as a thin film and data collection via ATR mode. IR spectra of complexes 22 and 31 were collected using a Bruker Tensor 37 FTIR with OPUS 6.5 software using samples prepared by pressing into a pellet with KBr salt. MS data was collected on a Bruker BioTOF II ESI/TOF-MS and an Agilent 7200 GC/QTOF-MS. UV-Visible spectra were collected at room temperature on a Cary 300 Bio UV-Visible spectrophotometer.

X-ray data for compounds 14, 15, and 28 were collected using a Bruker Photon III CPAD diffractometer for data collection at 125(2) K using Mo Kα radiation (normal parabolic mirrors). The data intensity was corrected for absorption and decay (SADABS). Final cell constants were obtained from least-squares fits of all measured reflections and the structure was solved and refined using SHELXL-2014/7.39. All non-hydrogen atoms were refined with anisotropic displacement parameters. All hydrogen atoms were geometrically placed. Details regarding refined data and cell parameters for specific structures are available in sections below.

Single crystal X-ray structure determinations for the rest of the compounds were performed by mounting a single crystal onto the tip of a 0.5 mm MiTeGen loop and mounted on a Bruker APEX II CCD diffractometer for a data collection at 130 K using Mo Kα radiation (normal parabolic mirrors). The data intensity was corrected for absorption and decay (SADABS). All calculations were performed using Pentium computers using the current SHELXTL suite of programs. All non-hydrogen atoms were refined with anisotropic displacement parameters. All hydrogen atoms were geometrically placed or were placed in ideal positions and refined as riding atoms with relative isotropic displacement parameters. Disordered components and hydrogens are omitted in ORTEP drawings for clarity. Data collection and structure solution were conducted at the X-Ray Crystallographic S10 Laboratory, supervised by Dr. Victor Young Jr. at the University of Minnesota. Details regarding refined data and cell parameters are available in Tables 1-12.

Bond length calculations were performed using Spartan Student V9 on a Mac computer following the simple equilibrium geometry calculations developed by Paton, Houk, and Garg. The structures were minimized using Molecular Mechanics, and then further minimized using Density Functional Theory B3LYP/6-31G* in the gas phase.

Bond length calculations of 7-azaindole and 7-azaindolyne are summarized in FIG. 19 and FIG. 20.

### General Procedure A for the Synthesis of Brominated Precursors

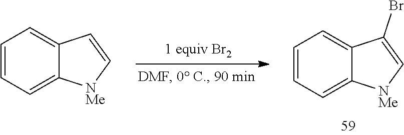 [SMILES: Cn1ccc2ccccc21 ; Cn1cc(Br)c2ccccc21]

The bromination of precursors using bromine was performed according to the procedure reported in the literature with minor modifications. A solution of Br₂ (0.410 mL, 8.00 mmol, 1 equiv) in anhydrous DMF (40 mL) was prepared. A separate round bottom flask was charged with 1-methyl-1H-indole (1.05 g, 8.00 mmol, 1 equiv) and anhydrous DMF (40 mL) and cooled in an ice bath. The Br₂ solution was added slowly to the indole flask and the reaction was allowed to stir at 0° C. Once the reaction was complete by TLC (90 min), ice was added to the reaction mixture. The mixture was diluted with EtOAc (200 mL) and washed with 1M aqueous LiCl solution (200 mL), dried (Na₂SO₄), filtered and concentrated. Purification by column chromatography (20% EtOAc/Hex) yielded compound 59 in 77% yield. The material obtained from this method provided identical 1H NMR spectra to those reported in the literature.

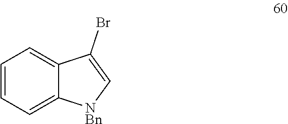 [SMILES: Brc1cn(Cc2ccccc2)c2ccccc12]

General procedure A was followed to yield compound 60 in 82% yield. The material obtained from this method provided identical 1H NMR spectra to those reported in the literature.

### General Procedure B for the Synthesis of Brominated Precursors

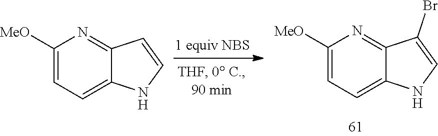 [SMILES: COc1ccc2[nH]ccc2n1 ; COc1ccc2[nH]cc(Br)c2n1]

The bromination of precursors using N-bromosuccinimide (NBS) was performed according to the procedure reported in the literature with minor modifications.⁷ To a solution of 5-methoxy-1H-pyrrolo[2,3-b]pyridine (2.0 g, 13.5 mmol, 1 equiv) in tetrahydrofuran (40 mL) was added N-bromosuccinimide (2.40 g, 13.5 mmol, 1 equiv) at 0° C. Completion of the reaction was monitored by TLC. After 90 minutes at 0° C., the reaction was quenched with 30% aqueous Na₂S₂O₃ (200 mL) and extracted with EtOAc (2×200 mL). The combined organic layers were washed with water (200 mL), dried with Na₂SO₄ and concentrated. The residue was triturated with EtOAc/diethyl ether (1:1) to give compound 61 in 87% yield. ¹H NMR (500 MHz, CDCl₃) δ 8.43 (br s, 1H), 7.56 (dd, J=8.8, 2.6 Hz, 1H), 7.35-7.32 (s, 1H), 6.66 (dd, J=8.8, 1.6 Hz, 1H), 4.05 (d, J=2.2 Hz, 3H). ¹³C{¹H} NMR (126 MHz, CDCl₃) δ 161.03, 139.90, 125.64, 124.14, 122.54, 107.26, 91.54, 53.68, 53.66. IR (cm⁻¹) 3406, 3107, 2944, 2835, 1709, 1606, 1487, 1444, 1333, 1311, 1202, 1174, 1122, 1076, 1028, 766, 731, 637, 617, 568, 511. HRMS (EI): 225.9742 and 227.9721 calculated for C₈H₇BrN₂O [M], found 225.9738 and 227.9721.

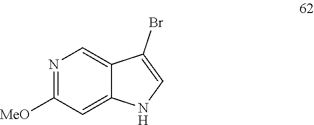 [SMILES: COc1cc2[nH]cc(Br)c2cn1]

General procedure B was followed to yield compound XX in 90% yield. ¹H NMR (500 MHz, CDCl₃) δ 8.44 (d, J=0.9 Hz, 1H), 8.18 (s, 1H), 7.11 (d, J=2.3 Hz, 1H), 6.65 (d, J=0.9 Hz, 1H), 3.99 (s, 3H). ¹³C{¹H} NMR (126 MHz, CDCl₃) δ 161.46, 143.23, 139.26, 124.18, 120.87, 90.72, 89.70, 54.33. IR (cm⁻¹) 3074, 3007, 2953, 2879, 2766, 2245, 2170, 2138, 2062, 2009, 1996, 1976, 1961, 1636, 1622, 1580, 1412, 1337, 1314, 1261, 1054, 944, 908, 847, 778, 679, 631, 621, 580, 550, 516, 497, 458, 446, 432, 417, 410. HRMS (EI) 225. 9742 and 227.9721 calculated for C₈H₇BrN₂O, found 225.9734 and 227.9718.

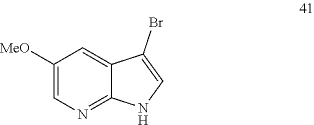 [SMILES: COc1cnc2[nH]cc(Br)c2c1]

Compound 41 was prepared via reported conditions in 56% yield. The material obtained from this method provided identical 1H NMR spectra to those reported. ¹H NMR (500 MHz, CDCl₃) δ 9.38 (s, 1H), 8.10 (d, J=2.7 Hz, 1H), 7.35 (d, J=2.7 Hz, 1H), 7.32 (d, J=2.5 Hz, 1H), 3.92 (s, 3H).

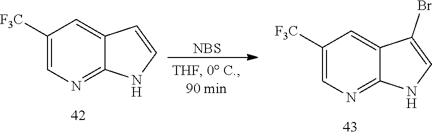

Compound 43 was prepared via reported conditions in 91% yield. The material obtained from this method provided identical 1H NMR and ¹⁹F{¹H} spectra to those reported. ¹H NMR (500 MHz, CDCl₃) δ 9.25 (s, 1H), 8.62 (d, J=2.6 Hz, 1H), 8.17 (d, J=2.1 Hz, 1H), 7.49 (d, J=2.5 Hz, 1H). ¹⁹F{¹H} NMR (471 MHz, CDCl₃) δ −60.16 (s).

General Procedure for Methyl Protection of Brominated 7-azaindoles

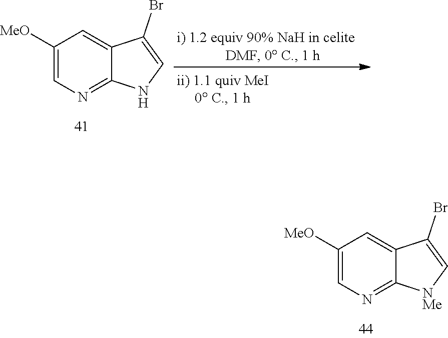 [SMILES: COc1cnc2[nH]cc(Br)c2c1 ; COc1cnc2c(c1)c(Br)cn2C]

The methyl protection of brominated azaindoles was performed according to the procedure reported in the literature by Penning et al. with minor modifications. 41 (1.3 g, 4.91 mmol, 1 equiv) was dissolved in anhydrous DMF (20 mL) and then cooled in an ice bath. NaH (157 mg, 90% in celite, 5.89 mmol, 1.2 equiv) was added and the mixture was stirred for 1 h at 0° C. MeI (336 μL, 5.4 mmol, 1.1 equiv) was then added and the mixture was stirred for an additional 1 h at 0° C. Water (50 mL) was carefully added to the reaction mixture while still in the ice bath to quench the reaction. The product was extracted with EtOAc (3×50 mL) and the combined organic layers were washed with 1M LiCl (3×50 mL), dried with Na₂SO₄, filtered and concentrated under vacuum. Column chromatography (hexane:EtOAc 5:1) afforded 44 in 82% yield (1.12 g).

NOTE: The hazards associated with the thermal decomposition of chemically incompatible NaH and DMF have been detailed in the literature. Decomposition of NaH/DMF occurs at 76.1° C. To address these concerns, the reaction mixture and subsequent quenching was kept in an ice bath. Additionally, careful consideration was taken when increasing the scale of the reaction.

¹H NMR (500 MHz, CDCl₃) δ 8.12 (d, J=2.7 Hz, 1H), 7.29 (d, J=2.7 Hz, 1H), 7.18 (s, 1H), 3.90 (s, 3H), 3.85 (s, 3H). ¹³C{¹H} NMR (126 MHz, CDCl₃) δ 151.69, 142.16, 135.18, 128.29, 119.47, 109.10, 86.60, 56.22, 31.37. IR (cm⁻¹) 3112, 2999, 2939, 2907, 2833, 1602, 1565, 1514, 1494, 1431, 1400, 1369, 1353, 1282, 1218, 1180, 1147, 1031, 977, 849, 798, 779, 760, 677, 622, 572, 558, 497. HRMS (ESI⁺) 240.9971 and 242.9951 calculated for C₉H₉BrN₂O [M+H]⁺, found 240.9976 and 240.9956.

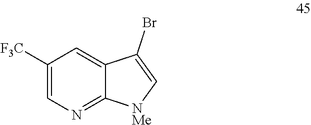 [SMILES: Cn1cc(Br)c2cc(C(F)(F)F)cnc21]

The general procedure was followed on 4.91 mmol (1.30 g) of compound 43 to give compound 45 in 82% yield (1.12 g).

¹H NMR (500 MHz, CDCl₃) δ 8.60 (d, J=2.1 Hz, 1H), 8.09 (d, J=2.1 Hz, 1H), 7.32 (s, 1H), 3.91 (s, 3H). ¹³C{¹H} NMR (126 MHz, CDCl₃) δ 148.08, 141.16 (q, J_{CF}=4 Hz), 130.15, 125.40 (q, J_{CF}=4 Hz), 124.81 (q, J_{CF}=271 Hz), 119.75 (q, J_{CF}=33 Hz), 119.05, 88.99, 31.69. ¹⁹F{¹H} NMR (471 MHz, CDCl₃): −59.99 (s). IR (cm⁻¹) 3123, 2945, 1612, 1564, 1522, 1501, 1445, 1409, 1373, 1340, 1308, 1265, 1238, 1141, 1111, 1168, 975, 901, 822, 811, 772, 726, 659, 619, 600, 580, 543. HRMS (ESI⁺) 278.9739 and 280.9718 calculated for C₉H₆BrF₃N₂ [M+H]⁺, found 278.9749 and 280.9725.

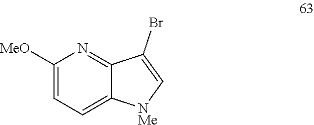 [SMILES: COc1ccc2c(n1)c(Br)cn2C]

The general procedure for methyl protection was followed to prepare compound 63 in 82% yield. ¹H NMR (500 MHz, CDCl₃) δ 7.52 (d, J=8.8 Hz, 1H), 7.18 (s, 1H), 6.66 (d, J=8.8 Hz, 1H), 4.05 (s, 3H), 3.77 (s, 3H). ¹³C{H} NMR (126 MHz, CDCl₃) δ 160.69, 139.95, 129.43, 125.52, 120.60, 88.96, 53.54, 33.64. IR (cm⁻¹) 3111, 3007, 2945, 2895, 1610, 1568, 1511, 1492, 1436, 1381, 1355, 1337, 1145, 1100, 1028, 863, 791, 774, 664, 605, 576, 513. HRMS (ESI⁺) 240.9977 and 242.9956 calculated for C₉H₉BrN₂O [M+Na]⁺, found 240.9973 and 242.9949.

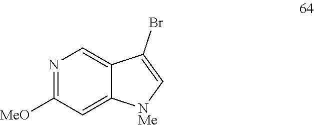 [SMILES: COc1cc2c(cn1)c(Br)cn2C]

The general procedure for methyl protection was followed to prepare compound 64 in 86% yield. ¹H NMR (400 MHz, CDCl₃) δ 8.40 (d, J=1.0 Hz, 1H), 6.94 (s, 1H), 6.55 (d, J=1.0 Hz, 1H), 4.00 (s, 3H), 3.67 (s, 3H). ¹³C{¹H} NMR (101 MHz, CDCl₃) δ 161.21, 143.91, 139.29, 128.64, 121.15, 88.92, 87.81, 54.23, 32.88. IR (cm⁻¹) 3093, 3030, 3003, 2949, 2856, 2051, 1971, 1798.8, 1623, 1529, 1472, 1460, 1439, 1349, 1281, 1169, 952, 902, 865, 786, 707, 627, 575, 551, 451, 440. HRMS (EI) 238.9815 and 240.9795 calculated for C₉H₉BrN₂O [M]^{+•} found 238.9813 and 240.9800.

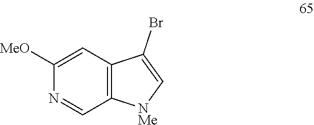 [SMILES: COc1cc2c(Br)cn(C)c2cn1]

The general procedure for methyl protection was followed to prepare compound 65 in 89% yield. ¹H NMR (500 MHz, CDCl₃) δ 8.32 (s, 1H), 7.16 (s, 1H), 6.81 (s, 1H), 3.98 (s, 3H), 3.82 (s, 3H). ¹³C{¹H} NMR (126 MHz, CDCl₃) δ 158.99, 136.35, 133.16, 130.96, 129.20, 95.94, 87.61, 54.46, 33.58. IR (cm⁻¹) 3112, 2999, 2943, 1619, 1560, 1514, 1355, 1244, 1226, 1151, 1123, 843, 790, 703, 613, 573, 448. HRMS (ESI⁺) 262.9796 and 264.9775 calculated for C₉H₉BrN₂O [M+Na]⁺, found 262.9792 and 264.9775.

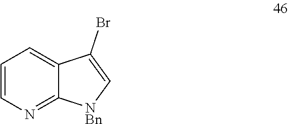 [SMILES: Brc1cn(Cc2ccccc2)c2ncccc12]

Compound 46 was prepared via reported conditions by Wang et al. Purification by column chromatography, (2:1 hexane to ethyl acetate) afforded compound 46 in 34% yield. Characterization data for this compound has been reported by Laha et al., The material obtained from this method provided identical 1H NMR spectra to those reported. ¹H NMR (500 MHz, CDCl₃) δ 8.39 (dd, J=4.7, 1.5 Hz, 1H), 7.87 (dd, J=7.9, 1.5 Hz, 1H), 7.30 (m, 3H), 7.23 (m, 2H), 7.20 (s, 1H), 7.16 (dd, J=7.9, 4.7 Hz, 1H), 5.48 (s, 2H).

### General Procedure for the Synthesis of Borylaryl Bromide Precursor

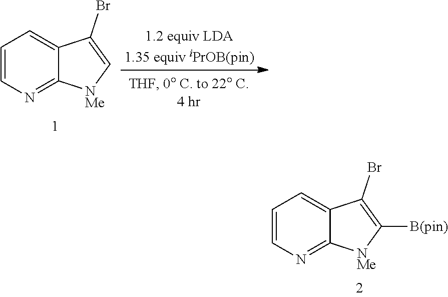

Synthesis of 3-bromo-1-methyl-7-azaindole (2)

An oven-dried flask equipped with a stir bar and sealed with a rubber septum under N₂ was charged with lithium diisopropylamide (LDA) solution (0.5 M in THF, 19.9 mmol, 1.2 equiv) and anhydrous THF (20 mL). The flask was cooled to 0° C. in an ice bath. A separate flask sealed with a rubber septum under N₂ was charged with anhydrous THF (25 mL), compound 3-bromo-1-methyl-1H-pyrrolo[2,3-b]pyridine (2.06 mL, 14.2 mmol, 1.0 equiv), and 2-isopropoxy-4,4,5,5-tetramethyl-1,3,2-dioxaborolane (ⁱPrOB(pin)) (3.92 mL, 19.2 mmol, 1.35 equiv). The flask was swirled to mix the solution, and the substrate mixture was transferred dropwise to the LDA flask via syringe. The substrate flask was rinsed with anhydrous THF (2×3 mL) and the rinse was added to the flask containing LDA. The flask was removed from the ice bath and stirring was continued. After 4 hrs, the reaction mixture was added to a separatory funnel charged with sat. NH₄Cl solution (20 mL) and EtOAc (20 mL). The layers were separated, and the organic layer was washed with water (20 mL), then brine (20 mL), dried (MgSO₄) and concentrated under reduced pressure. The resulting residue was purified by column chromatography using Teledyne ISCO CombiFlash (10% EtOAc in hexanes) to give compound 2 in 64% yield (3.05 g) as an off-white solid. The same general procedure was followed on 36.8 mmol scale (8.00 g) of compound 3-bromo-1-methyl-1H-pyrrolo[2,3-b]pyridine to give compound 2 in 54% (6.63 g) and 77% (9.54 g) yield (66% yield as average).

¹H NMR (500 MHz, CDCl₃) δ 8.42 (dd, J=4.6, 1.6 Hz, 1H), 7.89 (dd, J=7.9, 1.6 Hz, 1H), 7.10 (dd, J=7.9, 4.6 Hz, 1H), 4.05 (s, 3H), 1.40 (s, 12H). ¹³C{¹H} NMR (126 MHz, CDCl₃) δ 149.13, 145.96, 128.66, 120.44, 116.33, 100.76, 84.39, 31.39, 25.00. A signal for C¹—B was not observed. ¹¹B NMR (161 MHz, CDCl₃) δ 28.61. IR (cm⁻¹) 3051, 2978, 2936, 1594, 1564, 1507, 1459, 1407, 1374, 1327, 1307, 1258, 1214, 1142, 1083, 964, 947, 868, 846, 792, 770, 697, 670, 596, 550. HRMS (ESI⁺) 337.0718 and 339.0697 calculated for C₁₄H₁₈BBrN₂O₂[M+H]⁺, found 337.0723 and 339.0711.

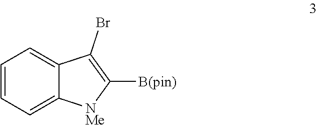

The general procedure was followed. Final purification by column chromatography (gradient 0-10% EtOAc in hexanes) afforded compound 3 in 58% yield. ¹H NMR (500 MHz, CDCl₃): 7.63 (dd, ³J_{HH} 8.0, 1.0 Hz, 1H), 7.37-7.29 (m, 2H), 7.18 (ddd, ³J_{HH} 8.0, 6.4, 1.5 Hz, 1H), 3.98 (s, 3H), 1.42 (s, 12H). ¹³C{¹H} NMR (126 MHz, CDCl₃): 139.2 (s), 127.7 (s), 124.2 (s), 120.3 (s), 120.1 (s), 109.8 (s), 102.8 (s), 84.0 (s), 32.8 (s), 24.9 (s). ¹¹B{¹H} NMR (161 MHz, CDCl₃): 28.5 (s). IR (cm⁻¹): 3058, 2978, 2935, 1742, 1612, 1510, 1464, 1432, 1381, 1327, 1310, 1285, 1265, 1232, 1143, 1110, 1084, 1009, 963, 945, 865, 845, 742, 694, 670, 595, 446. HRMS (EI) 335.0692 and 337.0672 calculated for C₁₅H₁₉BbrNO₂, found 335.0708 and 337.0656.

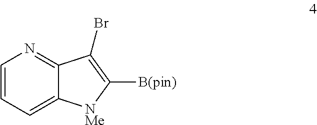

The general procedure was followed. Final purification by column chromatography (gradient 0-10% EtOAc in hexanes) afforded compound 4 in 63% yield. ¹H NMR (500 MHz, CDCl₃): 8.59 (dd, ³J_{HH} 4.4, 1.3 Hz, 1H), 7.66 (dd, ³J_{HH} 8.4, 1.3 Hz, 1H), 7.22 (dd, ³J_{HH} 8.4, 4.5 Hz, 1H), 3.98 (s, 3H), 1.43 (s, 12H). ¹³C{¹H} NMR (126 MHz, CDCl₃): 144.5 (s), 143.3 (s), 132.3 (s), 118.6 (s), 117.4 (s), 103.1 (s), (s), 84.4 (s), 32.9 (s), 24.9 (s). ¹¹B{¹H} NMR (161 MHz, CDCl₃): 28.6 (s). IR (cm⁻¹): 3045, 2978, 2938, 1597, 1507, 1510, 1446, 1415, 1385, 1321, 1283, 1260, 1203, 1141, 1085, 958, 868, 846, 787, 768, 698, 670, 581, 553.

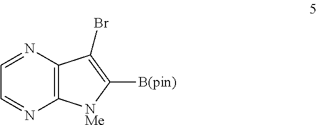

The general procedure was followed. Final purification by column chromatography (gradient 0-10% EtOAc in hexanes) afforded compound 5 in 16% yield. ¹H NMR (500 MHz, CDCl₃): 8.53 (d, ³J_{HH} 2.6 Hz, 1H), 8.36 (d, ³J_{HH} 2.6 Hz, 1H), 4.07 (s, 3H), 1.44 (s, 12H). ¹³C{¹H}NMR (126 MHz, CDCl₃): 142.6 (s), 139.5 (s), 139.4 (s), 136.9 (s), 101.1 (s), 84.7 (s), 31.3 (s), 24.9 (s). ¹¹B{¹H} NMR (161 MHz, CDCl₃): 28.5 (s). IR (cm⁻¹): 2979, 1542, 1505, 1455, 1408, 1381, 1331, 1265, 1221, 1139, 1081, 1047, 960, 847, 799, 945, 702, 669, 603, 587, 555, 432.

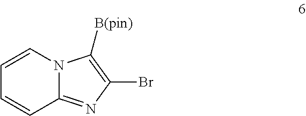

The general procedure was followed. Final purification by column chromatography (gradient 0-10% EtOAc in hexanes) afforded compound 6 in 43% yield. ¹H NMR (500 MHz, CDCl₃): 8.88 (dd, ³J_{HH} 6.8, 1.2 Hz, 1H), 7.60 (dd, ³J_{HH} 9.0, 1.2 Hz, 1H), 7.31 (m, 1H), 6.90 (dd, ³J_{HH} 6.9, 1.3 Hz, 1H), 1.42 (s, 12H).

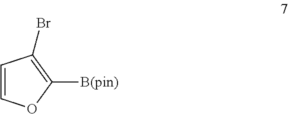

The general procedure was followed. Final purification by column chromatography (gradient 0-10% EtOAc in hexanes) afforded compound 7 as a pale-yellow solid in 53% yield. ¹H NMR (500 MHz, CDCl₃): δ 7.54 (d, J=1.8 Hz, 1H), 6.50 (d, J=1.8 Hz, 1H), 1.36 (s, 12H). ¹³C{¹H} NMR (126 MHz, CDCl₃): δ 147.80, 115.05, 113.83, 84.58, 24.92. ¹¹B{¹H} NMR (161 MHz, CDCl₃): δ 27.04.

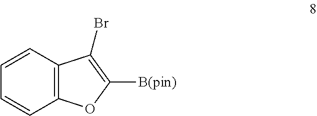

The general procedure was followed. Final purification by column chromatography (gradient 0-10% EtOAc in hexanes) afforded compound 8 as a pale-yellow solid in 86% yield. ¹H NMR (400 MHz, CDCl₃): δ 7.59 (dd, J=7.7, 1.3 Hz, 1H), 7.55 (d, J=8.3 Hz, 1H), 7.40 (ddd, J=8.4, 7.1, 1.4 Hz, 1H), 7.34-7.29 (m, 1H), 1.41 (s, 12H). ¹³C{¹H} NMR (126 MHz, CDCl₃): δ 156.69, 128.04, 127.23, 123.51, 120.78, 112.34, 111.51, 85.01, 24.96. ¹¹B{¹H} NMR (161 MHz, CDCl₃): δ 27.65.

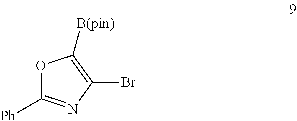

The general procedure was followed. Final purification by column chromatography (gradient 0-10% EtOAc in hexanes) afforded compound 9 as a pale-yellow solid. ¹H NMR (500 MHz, CDCl₃): δ [8.15-7.97 (m) and 7.51-7.42 (m) together total 5H], 1.39 (s, 12H).

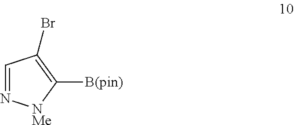

The general procedure was followed with minor modifications. NOTE: The product was observed to decompose slowly (˜6 hr) at room temperature therefore the product was stored in the freezer between work up and purification. Additionally, the product was stored in the glovebox freezer when not in use. Final purification by column chromatography (gradient 0-10% EtOAc in hexanes) afforded compound 10 as a pale-yellow solid in 39% yield. ¹H NMR (400 MHz, CDCl₃): δ 7.44 (s, 1H), 4.04 (s, 3H), 1.35 (s, 12H).

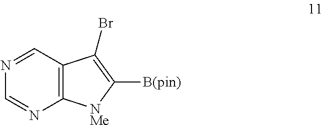

The general procedure was followed with minor modifications. The product was stored in the glovebox freezer when not in use. Final purification by washing crude product mixture with cold hexanes afforded compound 11 as a tan solid in 73% yield. ¹H NMR (400 MHz, CDCl₃): δ 8.94 (s, 1H), 8.93 (s, 1H), 4.01 (s, 3H), 1.41 (s, 12H). ¹³C{¹H} NMR (126 MHz, CDCl₃): δ 153.41, 151.83, 150.01, 118.80, 100.57, 85.63, 31.20, 24.84. ¹¹B{¹H} NMR (161 MHz, CDCl₃) δ 28.67. IR (cm⁻¹) 2976, 2942, 2240, 1581, 1557, 1507, 1487, 1442, 1407, 1381, 1372, 1351, 1331, 12912, 1267, 1212, 1109, 1081, 967, 876, 863, 784, 695, 667, 646, 628, 586, 550, 449, 406. HRMS (ESI⁺) 389.0643 and 391.0622 calculated for C₁₃H₁₇BBrN₃O₂[M+Na]⁺, found 360.0495 and 362.0474.

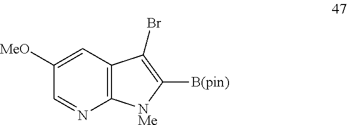

The general procedure was followed on a 4.98 mmol (1.20 g) scale of 44 to afford compound 47 in 69% yield (1.26 g). ¹H NMR (500 MHz, CDCl₃) δ 8.18 (d, J=2.7 Hz, 1H), 7.28 (d, J=2.8 Hz, 1H), 4.01 (s, 3H), 3.89 (s, 3H), 1.39 (s, 12H). ¹³C{¹H} NMR (126 MHz, CDCl₃) δ 151.80, 144.71, 137.95, 119.89, 109.15, 99.50, 84.29, 56.31, 31.48, 24.98. A signal for C¹—B was not observed. ¹¹B NMR (161 MHz, CDCl₃) δ 28.49. IR (cm⁻¹) 2976, 2935, 2837, 1563, 1505, 1451, 1402, 1378, 1314, 1235, 1208, 1170, 1136, 1083, 1031, 984, 963, 892, 852, 820, 765, 711, 695, 670, 581, 521. HRMS (ESI⁺) 389.0643 and 391.0622 calculated for C₁₅H₂₀BBrN₂O₃ [M+Na]⁺, found 389.0636 and 391.0631.

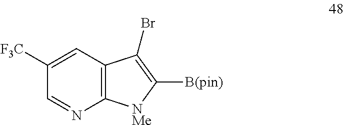

The general procedure was followed on a 2.69 mmol (750 mg) scale of compound 45 to afford compound 48 in 71% yield (770 mg). ¹H NMR (500 MHz, CDCl₃) δ 8.64 (d, J=2.1 Hz, 1H), 8.14 (d, J=2.1 Hz, 1H), 4.07 (s, 3H), 1.41 (s, 12H). ¹³C{¹H} NMR (126 MHz, CDCl₃) δ 150.13, 142.74 (q, J_{CF}=3.7 Hz), 126.45 (q, J_{CF}=3.9 Hz), 124.82 (q, J_{CF}=271.6 Hz), 119.63 (q, J_{CF}=32.6 Hz), 119.35, 101.64, 84.73, 31.71, 24.99. A signal for C¹—B was not observed. ¹¹B NMR (161 MHz, CDCl₃) δ 28.36. ¹⁹F{¹H} NMR (471 MHz, CDCl₃) δ −59.97. IR (cm⁻¹) 2981, 2937, 1608, 1561, 1515, 1459, 1381, 1343, 1314, 1258, 1222, 1143, 1121, 1083, 973, 909, 850, 779, 735, 800, 659, 604. HRMS (ESI⁺) 405.0591 and 407.0571 calculated for C₁₅H₁₇BBrF₃N₂O₂ [M+H]⁺, found 405.0608 and 407.0586.

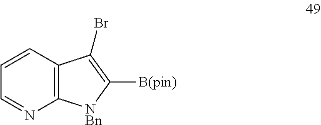

The general procedure was followed on a 4.88 mmol (1.40 g) scale of compound 46 to afford compound 49 as a tan solid in 42% yield (840 mg). ¹H NMR (500 MHz, CDCl₃) δ 8.42 (dd, J=4.6, 1.6 Hz, 1H), 7.92 (dd, J=8.0, 1.5 Hz, 1H), 7.22-7.12 (m, 4H), 7.06 (d, J=7.5 Hz, 2H), 5.84 (s, 2H), 1.25 (s, 12H) ¹³C{¹H} NMR (126 MHz, CDCl₃) δ 149.46, 146.48, 139.62, 128.99, 128.63, 127.31, 127.09, 120.63, 117.00, 101.93, 84.64, 47.33, 25.04. A signal for C¹—B was not observed. ¹¹B NMR (161 MHz, CDCl₃) δ 28.39. IR (cm⁻¹) 3031, 2977, 2933, 1593, 1564, 1506, 1483, 1446, 1379, 1357, 1328, 1288, 1268, 1243, 1214, 1140, 1108, 1076, 1031, 961, 940, 865, 845, 792, 770, 732, 696, 669, 639, 602, 569, 456. HRMS (ESI⁺) 413.1030 and 415.1043 calculated for C₂₀H₂₂BBrN₂O₂[M+H]⁺, found 413.1031 and 415.1030.

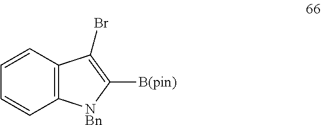

The general procedure was followed to afford compound 66 in 58% yield. H NMR (500 MHz, CDCl₃) δ 7.68 (d, J=8.0 Hz, 1H), 7.26 (m, 8H), 7.04 (d, J=7.0 Hz, 2H), 5.70 (s, 2H), 1.31 (s, 12H). ¹³C{¹H} NMR (126 MHz, CDCl₃) δ 139.10, 138.91, 128.56, 128.06, 127.17, 126.34, 124.59, 120.49 (d, J=9.1 Hz), 110.36, 49.32, 24.79. ¹¹B{¹H} NMR (161 MHz, CDCl₃) δ 28.59. IR (cm⁻¹) 3061, 2977, 1608, 1510, 1481, 1452, 1374, 1352, 1288, 1266, 1182, 1139, 1118, 1097, 1075, 1028, 991, 961, 942, 907, 864, 843, 727, 692, 669, 635, 578, 425. HRMS (ESI⁺) 412.1078 and 414.1057 calculated for C₂₁H₂₃BbrNO₂ [M+H]⁺, found 412.1067 and 414.1072.

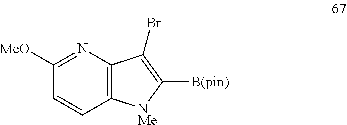

The general procedure was followed afford compound 67 in 68% yield. ¹H NMR (500 MHz, CDCl₃) δ 7.54 (d, J=8.9 Hz, 1H), 6.69 (d, J=8.9 Hz, 1H), 4.06 (s, 3H), 3.93 (s, 3H), 1.39 (s, 13H). ¹³C{¹H} NMR (126 MHz, CDCl₃) δ 160.80, 139.87, 128.94, 121.04, 108.79, 101.69, 84.23, 53.63, 33.32, 25.00. ¹¹B{¹H} NMR (161 MHz, CDCl₃) 28.48. IR (cm⁻¹) 2979, 2947, 1600, 1571, 1445, 1411, 1317, 1227, 1167, 1106, 1088, 1029, 994, 964, 801, 764, 698, 649, 592, 525, 501, 469. HRMS (ESI⁺) 389.0648 and 391.0607 calculated for C₁₅H₂₀BBrN₂O₃ [M+Na]⁺, found 389.0480 and 391.0621.

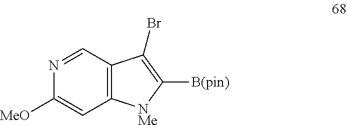

The general procedure was followed to afford compound 68 in 57% yield. ¹H NMR (400 MHz, CDCl₃) δ 8.45 (d, J=0.9 Hz, 1H), 6.50 (d, J=0.9 Hz, 1H), 3.99 (s, 4H), 3.81 (s, 4H), 1.38 (s, 15H). ¹³C{¹H} NMR (126 MHz, CDCl₃) δ 162.05, 146.26, 140.91, 121.82, 102.70, 87.28, 84.27, 54.30, 32.77, 24.95, 24.92. ¹¹B {¹H} (128 MHz, CDCl₃) δ 27.40. IR (cm⁻¹) 2976, 2933, 1762, 1618, 1564, 1514, 1418, 1379, 1345, 1312, 1285, 1268, 1201, 1166, 1107, 1079, 983, 962, 950, 913, 883, 856, 843, 813, 771, 831, 692, 626, 578, 560, 520, 451. HRMS (EI-MS) 366.0750 and 368.0730 calculated for C₉H₈BrN₂O [M], found 366.0732 and 368.0716.

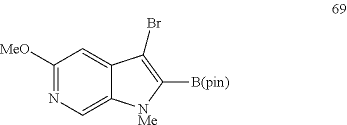

The general procedure was followed to yield compound 69 as in 74% yield. ¹H NMR (500 MHz, CDCl₃): δ 8.37 (s, 1H), 6.82 (s, 1H), 3.97 (two overlapping singlets, 6H), 1.40 (s, 12H). ¹³C{¹H} NMR (126 MHz, CDCl₃): δ 158.90, 136.23, 133.55, 130.14, 99.75, 95.85, 84.65, 54.58, 33.29, 25.01. A signal for C¹—B was not observed. ¹¹B{¹H} NMR (161 MHz, CDCl₃) δ 28.54. HRMS (ESI⁺) 367.0879 and 369.0934 calculated for C₁₅H₂₀BBrN₂O₃[M+H]⁺, found 367.0763 and 369.0955.

### General Procedure for the Synthesis of Metal-Aryne Complexes

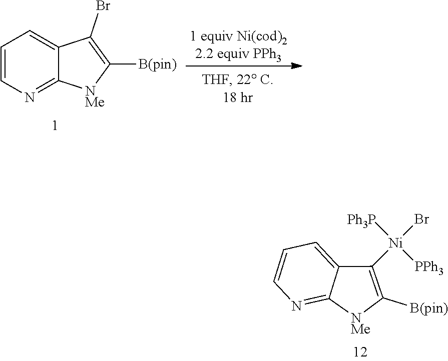

Synthesis of PPh₃ σ-aryl Complex 12

In a glovebox under N₂ atmosphere, Ni(COD)₂ (200 mg, 0.727 mmol, 1 equiv), precursor 1 (270 mg, 0.73 mmol, 1.1 equiv) and PPh₃ (420 mg, 1.6 mmol, 2.2 equiv) were added to a 20 mL vial and dissolved in anhydrous THF (5 mL, 145 mM). The reaction was stirred at room temperature for 18 hours. If precipitation occurred after 18 hours, the solid was collected by filtration through a frit. If the reaction mixture was still homogenous after 18 hours solvent was evaporated to approximately 1 mL and the desired complex was precipitated by adding Et₂O dropwise while stirring. The precipitated solid was subsequently collected by filtration through a frit. The solid collected over the frit was washed with Et₂O until the eluent ran clear (5-10 mL). The solid was collected from the frit and residual solid was dissolved using DCM. The solid was evaporated to dryness to give complex 12 as an orange powder in 62% yield (416 mg). The same procedure was run on a 2.6 mmol scale of Ni(COD)₂ to yield 62% (1.48 g) of complex 12.

Note: No clear peaks were observed via ¹¹B NMR analysis of all nickel complexes containing a B(pin) group. This is consistent with what is observed with similar complexes in the literature.

¹H NMR (400 MHz, CDCl₃) δ 8.04 (d, J=4.6 Hz, 1H), 7.86 (d, J=7.7 Hz, 1H), 7.69-7.38 (m, 11H), 7.38-7.23 (m, 8H), 7.16 (t, J=7.6 Hz, 11H), 6.46 (dd, J=7.9, 4.8 Hz, 1H), 3.30 (s, 3H), 1.28 (s, 12H). ¹³C{H} NMR (126 MHz, CDCl₃) δ 143.28, 134.88 (t, J_{C-P}=4.2 Hz), 132.30 (t, J_{C-P}=20.7 Hz), 131.88, 129.59, 127.38 (t, J_{C-P}=4.5 Hz), 112.75, 83.30, 30.65, 25.41. Signals for C¹—B, C²—Ni, and bridgehead carbons C³ and C⁸ were not observed. ³¹P{¹H}NMR (162 MHz, CDCl₃) δ 21.39. HRMS (ESI⁺) 557.1726 calculated for C₅₀H₄₈BBrN₂NiO₂P₂ [M−Br—PPh₃]⁺, found 557.1722. 839.2638 calculated for C₅₀H₄₈BBrN₂NiO₂P₂[M−Br]⁺, found 839.2640.

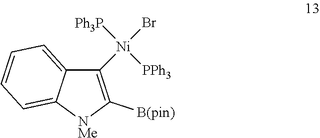

The general procedure was followed to give complex 13 in a 48% yield. ¹H NMR (400 MHz, CDCl₃): δ 7.79 (d, J=7.9 Hz, 1H), 7.52 (s, 10H), 7.33-7.22 (m, 8H), 7.15 (d, J=7.7 Hz, 12H), 6.92 (t, J=7.5 Hz, 1H), 6.74 (d, J=8.2 Hz, 1H), 6.54 (t, J=7.4 Hz, 1H), 3.20 (s, 3H), 1.27 (s, 12H). ¹³C{¹H} NMR (101 MHz, CDCl₃): δ 134.88, 132.52, 129.20, 127.10, 124.87, 121.72, 116.37, 108.49, 82.87, 77.36, 77.04, 76.72, 31.92, 25.31. ³¹¹P{¹H} NMR (162 MHz, CDCl₃) δ 21.00 (s). HRMS (ESI⁺) 838.2685 calculated for C₅₁H₄₉BbrNNiO₂P₂[M−Br]⁺, found 838.2659.

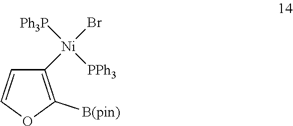

The general procedure was followed to give complex 14 in a 92% yield. Single crystals suitable for X-ray diffraction analysis were obtained from an NMR solution in C₆D₆. ¹H NMR (400 MHz, CDCl₃): δ 7.65 (m, 13H), 7.40-7.25 (m, 17H), 6.97 (d, J=1.6 Hz, 1H), 6.01 (d, J=1.5 Hz, 1H), 1.21 (s, 12H). ³¹P{¹H} NMR (162 MHz, CDCl₃): δ 22.89.

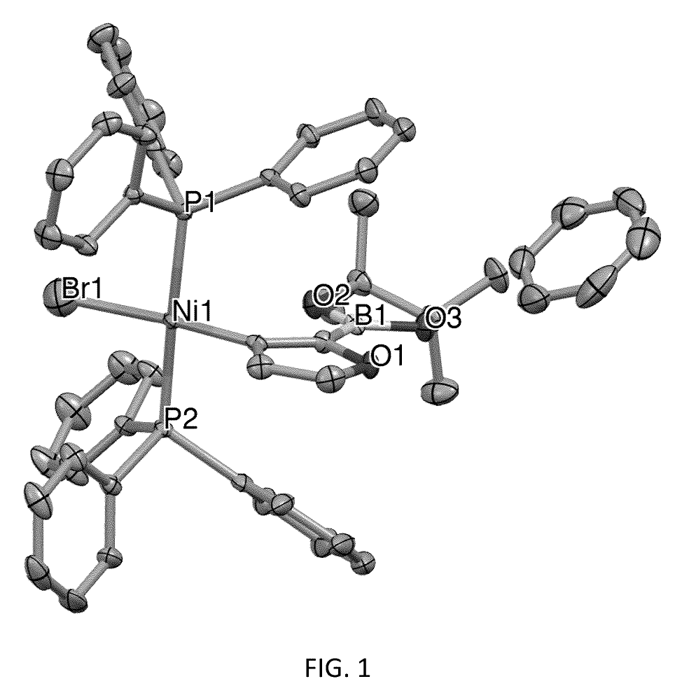

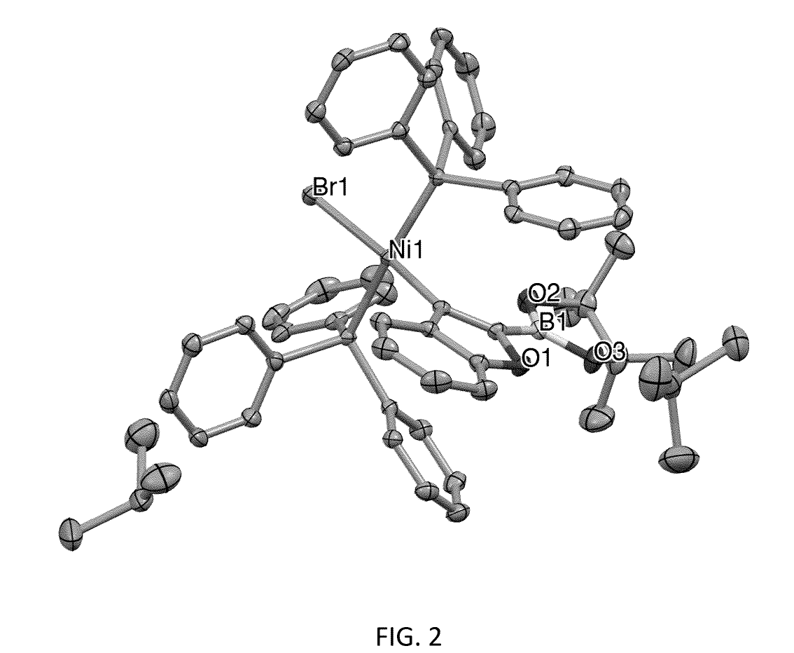

The crystal data for complex 14 is summarized below. The ORTEP drawing is depicted in FIG. 1.
Empirical formula: C₅₂H₅₀BbrNiO₃P₂
Formula weight: 934.29
Temperature: 150(2) K
Wavelength: 0.71073
Crystal system: Monoclinic
Space group: P 2₁/c
Unit cell dimensions (Å): a=20.8845(9)
   α=90°
   b=10.3461(5)
   β=105.253(2°)
   c=22.0837(11)
   γ=90°
Volume (Å³), 4603.6(4)
Z: 4
Density (calculated): 1.348 Mg/m³
Absorption coefficient: 1.401 mm⁻¹
F(000): 1936
Crystal color, morphology: yellow, cubic
Crystal size: 0.2×0.2×0.2 mm
Theta range for data collection: 1.912 to 30.540
Index ranges: −29≤h≤27, −14≤k≤14, −31≤1≤31
Reflections collected: 167618
Independent reflections: 14073 [R(int)=0.0603]
Observed reflections: 11476
Completeness to theta: 100.0%
Absorption correction: Multi-scan
Refinement method: Full-matrix least-squares on F2
Data/restraints/parameters: 14073/0/545
Goodness-of-fit on F2: 1.971
Final R indices [I>2sigma(I)]: R1=0.1195, wR2=0.3991
R indices (all data): R1=0.1385, wR2=0.4301
Extinction coefficient: n/a
Largest diff. peak and hole: 6.361 and −6.621 e Å³

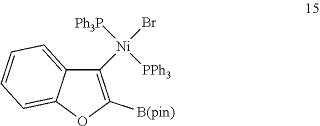

The general procedure was followed to give complex 15 in a 54% yield. Single crystals suitable for X-ray diffraction analysis were obtained from an NMR solution in CDCl₃. ¹H NMR (400 MHz, C₆D₆): δ 8.46-8.41 (m, 1H), 7.89 (s, 13H), 7.05 (d, J=7.9 Hz, 1H), 7.02-6.95 (m, 20H), 6.90 (dd, J=7.9, 6.6 Hz, 1H), 1.21 (s, 12H). ³¹P{¹H} NMR (162 MHz, C₆D₆): δ 24.26.

The crystal data for complex 15 is summarized below. The ORTEP drawing is depicted in FIG. 2.
Empirical formula: C₅₂H₄₈BBrCl₆NiO₃P₂
Formula weight: 1144.97
Temperature: 130(2) K
Wavelength: 0.71073 Å
Crystal system: Orthorhombic
Space group: Pbca
Unit cell dimensions: a=21.9255(10) Å
   α=900
   b=18.4128(9) Å
   β=90°
   c=25.8756(10) Å
   γ=90°
Volume: 10446.2(8) Å³
Z: 8
Density (calculated): 1.456 Mg/m³
Absorption coefficient: 1.546 mm
F(000): 4672
Crystal color, morphology: yellow, plate
Crystal size: 0.10×0.08×0.06 mm³
Theta range for data collection: 2.136 to 26.3880
Index ranges: −27≤h≤27, −23≤k≤22, −27≤1≤32
Reflections collected: 77893
Independent reflections: 10683 [R(int)=0.0663]
Observed reflections: 8269
Completeness to theta=25.242°: 99.9%
Absorption correction: Multi-scan
Refinement method: Full-matrix least-squares on F2
Data/restraints/parameters: 10683/0/599
Goodness-of-fit on F2: 1.022
Final R indices [I>2sigma(I)]: R1=0.0405, wR2=0.0919
R indices (all data): R1=0.0616, wR2=0.1027
Extinction coefficient: n/a
Largest diff. peak and hole: 0.948 and −0.749 e Å³

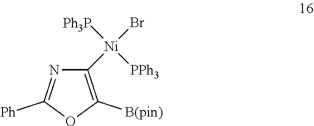

The general procedure was followed to give complex 16 in a 23% yield. ¹H NMR (400 MHz, CDCl₃): δ 7.70 (dh, J=6.9, 1.5 Hz, 11H), 7.51-7.43 (m, 2H), 7.33-7.26 (m, 7H), 7.23 (tt, J=8.2, 1.1 Hz, 14H), 1.35 (s, 12H). ³¹P{¹H} NMR (162 MHz, CDCl₃): δ 21.49.

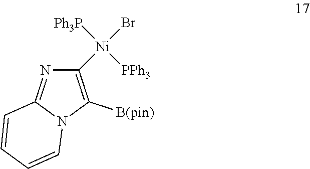

The general procedure was followed to give complex 17 in a 55% yield.

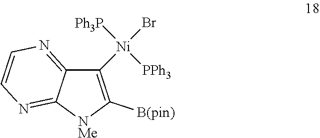

The general procedure was followed to give complex 18 in a 20% yield.

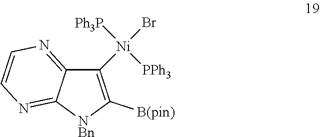

The general procedure was followed to give complex 19 in a 72% yield. Due to the heteroatoms in the aromatic ring there are likely bridged dimers or potential rotamers which cause duplication of NMR signals—both sets of signals are reported. ¹H NMR (400 MHz, CDCl₃): δ 8.13 (d, J=2.6 Hz, 2H), 8.09 (d, J=2.6 Hz, 1H), 7.82 (d, J=2.6 Hz, 2H), 7.81 (d, J=2.5 Hz, 1H), 7.70 (s, 29H), 7.35-7.27 (m, 6H), 7.22-7.14 (m, 25H), 7.07 (d, J=7.8 Hz, 31H), 4.96 (s, 2H), 4.94 (s, 4H), 1.30 (s, 21H), 1.27 (s, 12H). ³¹P NMR (162 MHz, CDCl3) δ 22.19, 20.65.

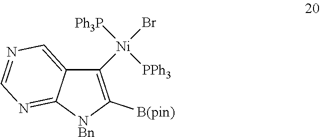

The general procedure was followed to give complex 20 in a 58% yield.

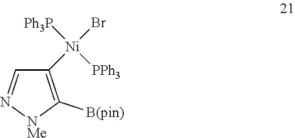

The general procedure was followed to give complex 21 in a 72% yield. ¹H NMR (400 MHz, CDCl₃): δ 7.57 (dh, J=6.8, 1.5 Hz, 12H), 7.36-7.30 (m, 9H), 7.25 (td, J=7.6, 7.1, 1.2 Hz, 10H), 3.42 (s, 3H), 1.19 (s, 12H). ³¹P{¹H} NMR (162 MHz, CDCl₃) δ 20.74.

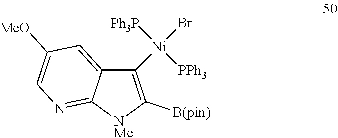

The general procedure was followed on 0.73 mmol scale of Ni(COD)₂ to give complex 50 in a 57% yield (397 mg). ¹H NMR (400 MHz, CDCl₃) δ 7.87 (d, J=2.8 Hz, 1H), 7.53 (m, 11H), 7.36-7.27 (m, 7H), 7.18 (m, 13H), 3.41 (s, 3H), 3.25 (s, 3H), 1.30 (s, 12H). ¹³C{¹H}NMR (126 MHz, CDCl₃) δ 148.92, 147.60, 135.00, 134.86, 132.33 (t, J_{C-P}=21.3 Hz), 129.63, 127.42 (t, J_{C-P}=4.8 Hz), 126.64, 121.30 (t, J_{C-P}=40.5 Hz), 112.79, 83.22, 55.62, 30.76, 25.53. A signal for C¹—B was not observed. ³¹P{¹H} NMR (162 MHz, CDCl₃) δ 21.26. HRMS (ESI⁺) 607.1831 calculated for C₅₁H₅₀BBrN₂NiO₃P₂[M-PPh₃-Br]⁺ found 607.1856. 869.2743 calculated for C₅₁H₅₀BBrN₂NiO₃P₂[M−Br]⁺ found 869.2754.

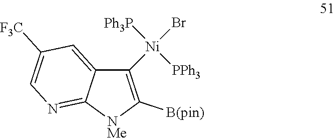

The general procedure was followed on 0.73 mmol scale of Ni(COD)₂ to give complex 51 in a 56% yield (400 mg). ¹H NMR (400 MHz, C₆D₆) δ 8.68 (s, 1H), 8.54 (d, J=1.9 Hz, 1H), 7.76 (s, 12H), 6.97 (m, 18H), 3.62 (s, 3H), 1.18 (s, 12H). ¹³C{¹H} NMR (126 MHz, CDCl₃) δ 153.07, 139.96 (q, J_{C-F}=3.0 Hz), 134.76, (t, J_{C-P}=4.8 Hz), 131.84 (t, J=21.7 Hz), 129.79, 129.55 (d, J=4.1 Hz), 127.49 (t, J=4.8 Hz), 125.95 (q, J_{C-F}=2.1 Hz), 115.52 (q, J=32.1 Hz), 83.58, 68.11, 25.41. ³¹P{¹H} NMR (162 MHz, C₆D₆) δ 21.82. ¹⁹F{¹H} NMR (376 MHz, C₆D₆) δ −59.09. HRMS (ESI⁺) 645.1600 calculated for C₅₁H₄₇BBrF₃N₂NiO₂P₂[M−PPh₃−Br]⁺ found 645.1627. 907.2511 calculated for C₅₁H₄₇BBrF₃N₂NiO₂P₂[M−Br]⁺ found 907.2555.

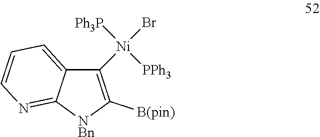

The general procedure was followed on 0.73 mmol scale of Ni(COD)₂ to give complex 52 in a 62% yield (451 mg). ¹H NMR (400 MHz, CDCl₃): 8.09 (d, J=4.8 Hz, 1H), 7.93 (d, J=7.7 Hz, 1H), 7.51 (m, 11H), 7.35-7.27 (m, 3H), 7.25-6.97 (m, 21H), 6.53 (dd, J=7.7, 4.7 Hz, 1H), 4.90 (s, 2H), 1.26 (s, 12H). ¹³C{¹H} NMR (126 MHz, CDCl₃) δ 134.81, 132.19, 129.58, 127.40, 25.15, 25.08. Note: Complex is completely insoluble in PhMe, benzene, acetonitrile, and THF while only minimally soluble in warm DCM and CHCl₃. Due to this minimal solubility and poor concentration, only PPh₃ and pinacol borane carbons are visible by ¹³C{¹H}NMR. ³¹P{¹H} NMR (162 MHz, CDCl₃) 21.39. HRMS (ESI⁺) 653.2039 calculated for C₅₆H₅₂BBrN₂NiO₂P₂[M−Br—PPh₃]⁺, found 653.2067. 915.2951 calculated for C₅₆H₅₂BBrN₂NiO₂P₂[M−Br]⁺, found 915.2979.

### Procedure for Preparation of TMEDA σ-Aryl Complexes.

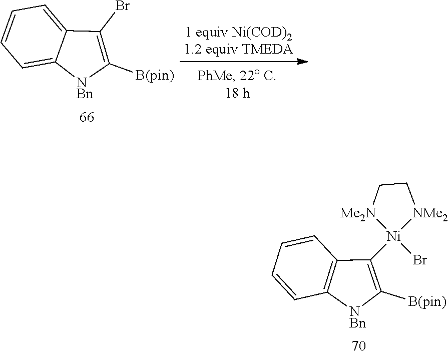

In a glovebox, to an oven-dried vial was added compound 66 (300 mg, 0.727 mmol, 1 equiv) and Ni(COD)₂ (200 mg, 0.727 mmol, 1 equiv). A solution of N,N,N′,N′-Tetramethylethylenediamine (TMEDA) (131 μL, 0.873, 1.2 equiv) in toluene (5 mL, 0.145 M) was prepared. The solution was added to the Ni(COD)₂ vial. The mixture was allowed to stir at 22° C. After 18 h, the solution had turned pink and the product began to precipitate as a pink solid. Pentane was added to the solution to crash out additional precipitate and the solid was collected on a Hirsch funnel to yield compound 70 in 84% yield. ¹H NMR (500 MHz, CDCl₃) 8.62 (d, J=7.6 Hz, 1H), 7.21-6.79 (overlapping peaks, indole and phenyl aromatic peaks, 9H), 6.21-4.84 (m, benzylic CH₂, 2H), 3.15-1.65 (overlapping TMEDA peaks, 16H), 1.42 (s, 12H). ¹³C{¹H}NMR (126 MHz, CDCl₃) 141.36, 141.07, 135.65, 128.00, 126.53, 126.27, 125.93, 122.10, 119.17, 117.51, 109.65, 83.21, 62.00-56.00 (TMEDA, broad peak), 49.31, 48.58, 25.26. C¹—B not observed. 11B{¹H} NMR 29.21. HRMS (ESI⁺) 506.2489 calculated for C₂₇H₃₉BBrN₃NiO₂ [M−Br]⁺, found 506.2471.

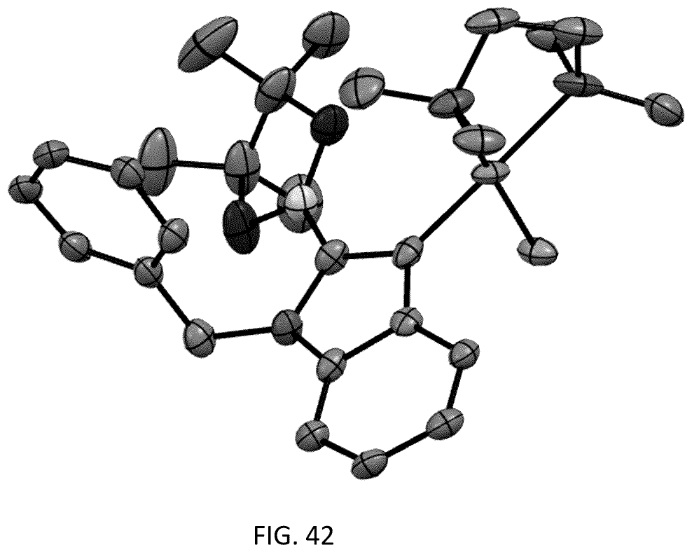

The crystal data for complex 70 is summarized below. The ORTEP drawing is depicted in FIG. 42.

The structure is the one suggested.

TABLE 1
Crystal data and structural refinement for complex 70.

| Empirical formula | C₂₇ H₃₉ B Br N₃ Ni O₂ | |
|---|---|---|
| Formula weight | 587.04 | |
| Temperature | 100(2) K | |
| Wavelength | 0.71073 Å | |
| Crystal system | Monoclinic | |
| Space group | P2₁/c | |
| Unit cell dimensions | a = 18.1403(7) Å | a = 90° |
| | b = 11.3083(5) Å | b = 108.867(2) ° |
| | c = 14.9475(6) Å | g = 90° |

| | |
|---|---|
| Volume | 2901.5(2) Å³ |
| Z | 4 |
| Density (calculated) | 1.344 Mg/m³ |
| Absorption coefficient | 2.073 mm⁻¹ |
| F(000) | 1224 |
| Crystal color, morphology | red, block |
| Crystal size | 0.150 × 0.100 × 0.100 mm³ |
| Theta range for data collection | 2.157 to 27.518° |
| Index ranges | −23 ≤ h ≤ 21, −14 ≤ k ≤ 14, −19 ≤ 1 ≤ 19 |
| Reflections collected | 42815 |
| Independent reflections | 6677 [R(int) = 0.0465] |
| Observed reflections | 5509 |
| Completeness to theta = 25.242° | 99.9% |
| Absorption correction | Semi-empirical from equivalents |
| Max. and min. transmission | 0.7456 and 0.6267 |
| Refinement method | Full-matrix least-squares on F² |
| Data/restraints/parameters | 6677/104/438 |
| Goodness-of-fit on F² | 1.064 |
| Final R indices [I > 2sigma(I)] | R1 = 0.0406, wR2 = 0.1091 |
| R indices (all data) | R1 = 0.0519, wR2 = 0.1169 |
| Extinction coefficient | n/a |
| Largest diff. peak and hole | 1.254 and −0.578 eÅ⁻³ |

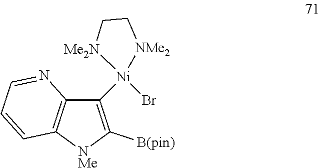

The general procedure was followed to give complex 71 in a 95% yield. ¹H NMR (500 MHz, CDCl₃) 8.53 (d, J=4.5 Hz, 1H), 7.29 (d, J=8.2 Hz, 1H), 6.95 (dd, J=8.2, 4.5 Hz, 1H), 3.85 (s, 3H), 2.62 (broad s, 12H), 2.35 (broad s, 4H), 1.55 (s, 12H). ¹³C{¹H} NMR (126 MHz, CDCl₃) 153.52, 141.33, 134.22, 115.85, 115.67, 83.38, 58.93, 49.20, 32.11, 25.40. ¹¹B{¹H} NMR (CDCl₃) 29.64. HRMS (ESI⁺) 431.2128 calculated for C₅₁H₅₀BBrN₂NiO₃P₂[M−Br]⁺ found 431.2130.

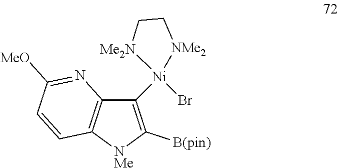

The general procedure was followed to give complex 72 in a 95% yield. ¹H NMR (500 MHz, CDCl₃) 8.53 (d, J=4.5 Hz, 1H), 7.29 (d, J=8.2 Hz, 1H), 6.95 (dd, J=8.2, 4.5 Hz, 1H), 3.85 (s, 3H), 2.62 (broad s, 12H), 2.35 (broad s, 4H), 1.55 (s, 12H). ¹³C{¹H} NMR (126 MHz, CDCl₃) 153.52, 141.33, 134.22, 115.85, 115.67, 83.38, 58.93, 49.20, 32.11, 25.40. ¹¹B{¹H} NMR (CDCl₃) 29.64. HRMS (ESI⁺) 431.2128 calculated for C₅₁H₅₀BBrN₂NiO₃P₂[M−Br]⁺ found 431.2130.

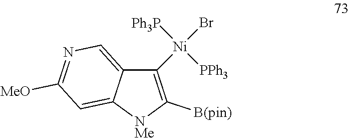

The general procedure was followed to give complex 73 in a 53% yield. ¹H NMR (400 MHz, CDCl₃) δ 8.70 (s, 1H), 7.60 (s, 14H), 7.33 (s, 2H), 7.17 (d, J=9.1 Hz, 14H), 5.89 (s, 1H), 3.86 (s, 3H), 3.10 (d, J=11.5 Hz, 3H), 1.25 (s, 12H). ¹³C{¹H} NMR (126 MHz, CDCl₃) δ 160.64, 148.29, 143.56, 134.91, 132.24 (t, J_{CP}=21.6 Hz), 129.50, 127.34, 85.99, 83.22, 54.17, 31.88, 25.14. ³¹P{¹H} NMR (162 MHz, CDCl₃) δ 21.66. HRMS (ESI⁺) 607.1832 calculated for C₄₅H₃₈BrN₂NiOP₂ [M−Br−PPh₃]⁺, found 607.1839.

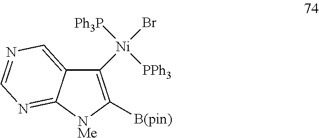

The general procedure was followed to give complex 74 in a 43% yield. There are likely bridged dimers or oligomers in solution where the additional heteroatoms in the aromatic ring displace the PPh₃ ligands to form various oligomers. This dynamic process may cause the observed broadening an underintegration of the PPh₃ signals in both the ¹H and ¹³C{¹H} NMR spectra. ¹H NMR (500 MHz, CDCl₃) δ 10.49-10.45 (s, 1H), 9.08 (s, 1H), 8.98-6.10 (br m, broadening of PPh₃ signals), 3.22 (s, 3H), 1.50 (s, 6H), 1.44 (s, 6H). ¹³C{¹H} NMR (126 MHz, CDCl₃) δ 152.91, 151.19, 150.29, 125.39, 84.11, 30.58, 25.89, 24.85. Signals for the PPh₃ carbons were broadened out and were not observable above the baseline noise. ³¹P{¹H} NMR (162 MHz, CDCl₃) δ 28.93. HRMS (ESI⁺) 578.1679 calculated for C₄₉H₄₉BBrN₃NiO₂P₂[M−Br−PPh₃]⁺, found 578.1651.

General Procedure for Ligand Exchange to DCPE (σ-aryl Complexes

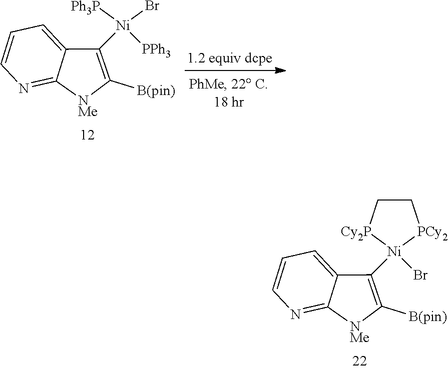

Synthesis of DCPE σ-aryl Complexes

In a glovebox under N₂ atmosphere, complex 12 (401 mg, 0.44 mmol, 1 equiv) and dcpe (221 mg, 0.52 mmol, 1.2 equiv) were added to a 20 mL vial and dissolved in PhMe (12.2 mL, 36 mM). The reaction was stirred at room temperature. If precipitation occurred after 18 hours, the solid was collected by filtration through a frit. If the reaction mixture was still homogenous after 18 hours solvent was evaporated to approximately 1 mL and the desired complex was precipitated by adding Et₂O dropwise while stirring. The precipitated solid was subsequently collected by filtration through a frit. The solid collected over the frit was washed with Et₂O until the eluent ran clear (5-10 mL). The solid was collected from the frit and residual solid was dissolved using DCM. The solid was evaporated to dryness to give complex 22 as a yellow-orange solid in >98% yield (354 mg). The general procedure was followed at 1.61 mmol scale of complex 12 to yield complex 22 in 91% yield (1.20 g). Single crystals suitable for X-ray crystallography were grown from a DCM solution of the complex with slow vapor diffusion of pentane at 22° C.

¹H NMR (400 MHz, CDCl₃): δ 8.24 (d, J=4.6 Hz, 1H), 8.17 (d, J=7.7 Hz, 1H), 6.82 (dd, J=7.7, 4.6 Hz, 1H), 4.01 (s, 3H), 2.82-0.73 (m, 60H). ¹³C{¹H} NMR (101 MHz, CDCl₃) δ 152.57 (d, J_{C-P}=4.6 Hz), 143.19, 133.90 (dd, J_{C-P}=90.9, 39.9 Hz), 133.03, 128.77, 128.75, 112.76, 82.99, 35.80, 35.69, 35.55, 35.48, 35.27, 34.66, 34.48, 34.09, 33.93, 32.25, 31.13, 30.40, 29.98, 29.96, 29.43, 29.33, 28.99, 28.76, 28.56, 28.52, 28.38, 28.33, 27.77, 27.72, 27.69, 27.64, 27.58, 27.55, 27.30, 27.22, 26.92, 26.83, 26.78, 26.71, 26.41, 26.21, 26.18, 26.14, 26.02, 25.80, 25.30, 24.93, 24.64, 24.27, 23.54, 23.36, 23.19, 19.68, 19.59, 19.52, 19.43. A signal for C¹—B was not observed. ³¹P{¹H} NMR (162 MHz, CDCl₃): δ 64.68 (d, Jₚₚ=28.5 Hz), 59.76 (d, Jₚₚ=28.6 Hz). HRMS (ESI⁺) 737.4046 calculated for C₄₀H₆₆BBrN₂NiO₂P₂[M−Br]⁺, found 737.4011.

Note: No clear peaks were observed via ¹¹B NMR analysis of all nickel complexes containing a B(pin) group. This is consistent with what is observed with similar complexes in the literature. Peaks in the alkyl region of ¹H NMR spectra often over-integrate due to residual solvent and/or inseparable dcpe-related impurities. The alkyl region of ¹³C{¹H} NMR spectra contain several overlapping multiplets so all peaks are reported individually.

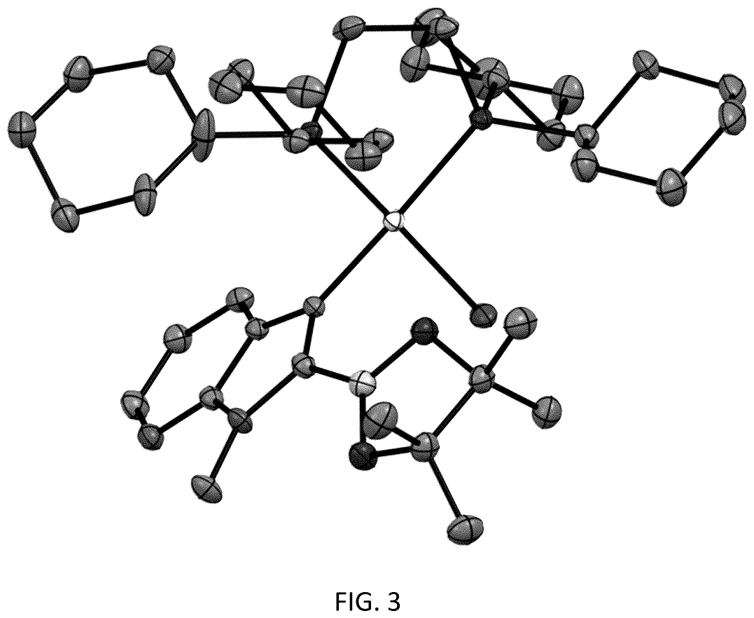

The crystal data for complex 22 is summarized below. The ORTEP drawing is depicted in FIG. 3.

The structure is the one suggested. Disorder of cyclohexane C37-C40 was modeled by selection of 4 strongest Q peaks and separation into 2 parts.

TABLE 2
Crystal data and structural refinement for complex 22.

| Empirical formula | C₄₁ H₆₈ B Br Cl₂ N₂ Ni O₂ P₂ | |
|---|---|---|
| Formula weight | 903 | |
| Temperature | 130(2) K | |
| Wavelength | 0.71073 Å | |
| Crystal system | Monoclinic | |
| Space group | P2₁/c | |
| Unit cell dimensions | a = 22.7039(8) Å | a = 90° |
| | b = 12.0060(4) Å | b = 104.983(2) ° |
| | c = 16.5889(5) Å | g = 90° |

| | |
|---|---|
| Volume | 4368.1(3) Å³ |
| Z | 4 |
| Density (calculated) | 1.373 Mg/m³ |
| Absorption coefficient | 1.591 mm⁻¹ |
| F(000) | 1904 |
| Crystal color, morphology | yellow, block |
| Crystal size | 0.109 × 0.095 × 0.040 mm³ |
| Theta range for data collection | 1.934 to 30.53300 |
| Index ranges | −32 ≤h ≤32, −17 ≤ k ≤ 16, −21 ≤ 1 ≤ 23 |
| Reflections collected | 45163 |
| Independent reflections | 13329 [R(int) = 0.0554] |
| Observed reflections | 9285 |
| Completeness to theta = 25.242° | 100.0% |
| Absorption correction | Semi-empirical from equivalents |
| Refinement method | Full-matrix least-squares on F² |
| Data / restraints / parameters | 13329 / 16 / 502 |
| Goodness-of-fit on F2 | 1.015 |
| Final R indices [I > 2sigma(I)] | R1 = 0.0508, wR2 = 0.1139 |
| R indices (all data) | R1 = 0.0872, wR2 = 0.1293 |
| Extinction coefficient | n/a |
| Largest diff. peak and hole | 1.191 and −1.514 e · Å⁻³ |

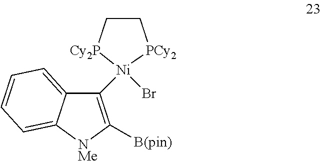

The general procedure was followed to give complex 23 in a 93% yield. ¹H NMR (400 MHz, CDCl₃): δ 7.96 (d, J=7.9 Hz, 1H), 7.14-7.06 (m, 2H), 6.88 (ddd, J=7.9, 6.4, 1.5 Hz, 1H), 3.91 (s, 3H), 2.86-0.72 (m, 61H). ¹³C{¹H} NMR (101 MHz, CDCl₃) δ 142.00, 136.73, 129.14, 128.33, 125.84, 121.74, 116.52, 109.03, 82.72, 35.71-35.22 (m), 34.57 (d, J_{CP}=22.1 Hz), 34.03 (d, J_{CP}=20.6 Hz), 32.43 (d, J_{CP}=17.1 Hz), 30.03, 29.03, 28.68, 28.41, 28.03-27.55 (m), 27.53-27.06 (m), 26.94 (d, J_{CP}=11.0 Hz), 26.71 (d, J_{CP}=8.0 Hz), 26.49, 26.24, 26.09, 24.30, 23.41 (t, J_{CP}=22.0 Hz), 19.40 (d, J_{CP}=14.3 Hz). ³¹P{¹H} NMR (162 MHz, CDCl₃): δ 63.33 (d, J=26.8 Hz), 58.49 (d, J=26.7 Hz).

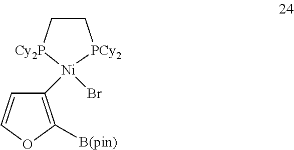

The general procedure was followed to give complex 24 in a 68% yield. ¹H NMR (400 MHz, CDCl₃): δ 7.60 (d, J=1.4 Hz, 1H), 6.27 (d, J=1.7 Hz, 1H), 2.57-0.91 (m, 60H). ³¹P{¹H} NMR (162 MHz, CDCl₃): δ 72.04 (d, J=30.1 Hz), 64.84 (d, J=30.1 Hz).

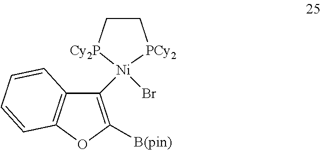

The general procedure was followed to give complex 25 in a 16% yield. ¹H NMR (400 MHz, CDCl₃): δ 7.89 (d, J=7.7 Hz, 1H), 7.42 (d, J=8.1 Hz, 1H), 7.16 (t, J=7.9 Hz, 1H), 7.07 (t, J=7.4 Hz, 1H), 2.48-0.81 (m, 60H). ³¹P{¹H} NMR (162 MHz, CDCl₃): δ 69.14 (d, J=31.3 Hz), 64.47 (d, J=31.4 Hz).

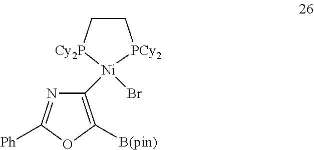

The general procedure was followed to give complex 26 in quantitative yield. ¹H NMR (500 MHz, CDCl₃): δ 8.11 (d, J=7.6 Hz, 2H), 7.33 (t, J=7.6 Hz, 2H), 7.24 (d, J=2.6 Hz, 1H), 2.57-2.10 (m, 6H), 1.93-1.44 (m, 27H), 1.33 (s, 13H), 1.30-0.79 (m, 11H). ¹³C{¹H}NMR (126 MHz, CDCl₃): δ 165.29 (d, J=5.8 Hz), 129.36, 128.18, 126.19, 83.06, 35.65 (d, J=25.5 Hz), 35.33, 35.15, 29.69 (d, J=2.2 Hz), 29.01 (d, J=2.9 Hz), 28.13 (d, J=3.2 Hz), 27.65-27.43 (m), 27.05 (d, J=10.1 Hz), 26.17 (d, J=21.0 Hz), 25.21, 24.62-24.13 (m), 20.34, 20.12 (d, J=12.4 Hz). ³¹P{¹H} NMR (162 MHz, CDCl₃) δ 78.34 (d, J=36.0 Hz), 67.61 (d, J=36.2 Hz).

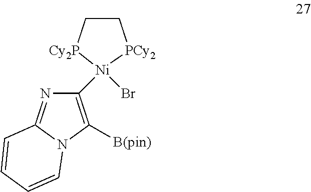

The general procedure was followed to give complex 27 in a 64% yield. Due to the heteroatoms in the aromatic ring there are likely bridged dimers or potential rotamers which cause duplication of NMR signals—both sets of signals are reported. ¹H NMR (400 MHz, CDCl₃): δ 8.86 (dt, J=6.7, 1.2 Hz, 1H), 7.46-7.16 (m, 3H), 6.90 (ddd, J=8.8, 6.6, 1.4 Hz, 1H), 6.49 (td, J=6.7, 1.4 Hz, 1H), 2.60-1.11 (m, 69H). ³³¹P{¹H} (162 MHz, CDCl₃): δ 70.36 (d, J=26.6 Hz), 67.93 (d, J=28.1 Hz), 62.75 (dd, J=41.1, 27.5 Hz).

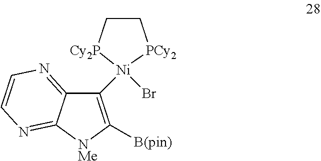

The general procedure was followed to give complex 28 in a 50% yield. Due to the heteroatoms in the aromatic ring there are likely bridged dimers or potential rotamers which cause duplication of NMR signals—both sets of signals are reported. Single crystals suitable for X-ray diffraction analysis were obtained from a THF solution layered with pentane. ¹H NMR (400 MHz, CDCl₃): δ 8.34 (dd, J=4.4, 1.5 Hz, 1H), 6.91 (dd, J=8.1, 4.4 Hz, 1H), 3.90 (d, J=3.2 Hz, 3H), 2.79-0.96 (m, 157H). ³¹P{¹H} (162 MHz, CDCl₃): δ 67.46 (d, J=30.1 Hz), 64.63 (d, J=31.6 Hz), 60.04 (dd, J=30.7, 18.7 Hz).

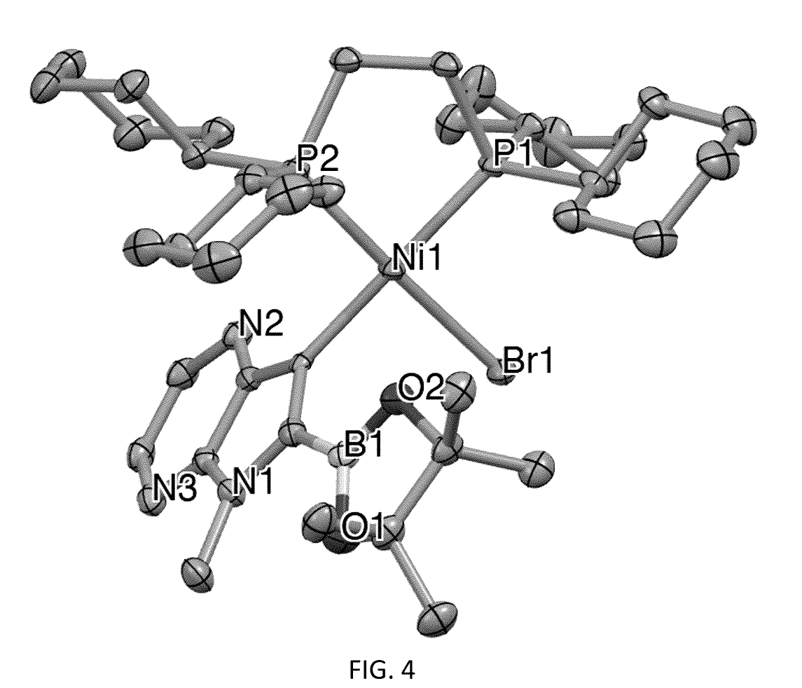

The crystal data for complex 28 is summarized below. The ORTEP drawing is depicted in FIG. 4.
Empirical formula: C₃₉H₆₅BBrN₃NiO₂P₂
Formula weight: 819.31
Temperature: 130(2) K
Wavelength: 0.71073 Å
Crystal system: Monoclinic
Space group: P2₁/n
Unit cell dimensions: a=12.5923(3) Å
   α=90°
   b=22.8765(6) Å
   β=111.7930(10°)
   c=15.1741(3) Å
   γ=90°
Volume: 4058.77(17) Å³
Z: 4
Density (calculated): 1.341 Mg/m³
Absorption coefficient: 1.578 mm⁻¹
F(000): 1736
Crystal color, morphology: yellow, plate
Crystal size: 0.100×0.100×0.070 mm³
Theta range for data collection: 1.956 to 27.118°
Index ranges: −16≤h≤15, −27≤k≤29, −16≤1≤19
Reflections collected: 39227
Independent reflections: 8959 [R(int)=0.0401]
Observed reflections: 7441
Completeness to theta=25.242°: 99.9%
Absorption correction: Multi-scan
Max. and min. transmission: 1.0000 and 0.8767
Refinement method: Full-matrix least-squares on F2
Data/restraints/parameters: 8959/0/447
Goodness-of-fit on F2: 1.133
Final R indices [I>2sigma(I)]: R1=0.0297, wR2=0.0823
R indices (all data): R1=0.0425, wR2=0.0989
Extinction coefficient: n/a
Largest diff. peak and hole: 0.810 and −0.559 e Å³

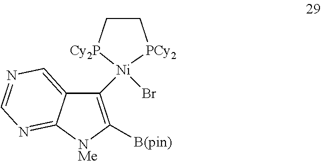

The general procedure was followed to give complex 29 in 91% yield. ¹H NMR (500 MHz, CDCl₃) δ 9.13 (s, 1H), 8.74 (s, 1H), 3.95 (m, 3H), 2.68 (m, 2H), 2.37 (d, J=10.2 Hz, 2H), 2.08-0.99 (m, overlapping with B(pin) methyl signal, 53H), 0.98-0.82 (m, 3H). ¹³C{¹H}NMR (126 MHz, CDCl₃) δ 155.19, 152.95, 151.40, 137.72 (m), 127.34, 35.66 (d, J_{CP}=18.6 Hz), 35.43 (d, J_{CP}=25.0 Hz), 34.77 (t, J_{CP}=18.8 Hz), 34.53, 31.84, 30.99, 30.06, 29.18, 28.88, 28.75, 28.26, 27.91, 27.83-27.10 (m, overlapping multiplets), 27.02-26.59 (m, overlapping multiplets), 26.17 (m, overlapping multiplets), 24.24, 23.45 (t, J_{CP}=22.3 Hz), 19.75 (m). ¹P{¹H} NMR (203 MHz, CDCl₃) δ 66.37 (d, Jₚₚ=31.1 Hz), 62.28 (d, J_{PP}=31.1 Hz). HRMS (ESI⁺) 738.3998 calculated for [M−Br]⁺, found 738.3979.

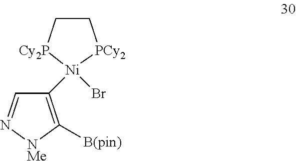

The general procedure was followed to give complex 30 in 85% yield. ¹H NMR (400 MHz, CDCl₃): δ 7.08 (s, 1H), 4.05 (s, 3H), 2.54-1.08 (m, 69H). ³¹P{¹H} (162 MHz, CDCl₃): δ 71.73 (d, J=29.5 Hz), 63.34 (d, J=29.5 Hz).

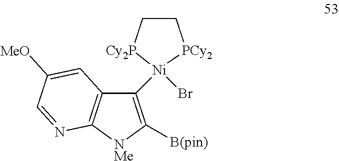

The general procedure was followed on 0.42 mmol scale of complex 50 to give complex 53 as an orange-yellow powder in 97% yield (344 mg). Single crystals suitable for X-ray crystallography were grown from evaporation of a DCM solution of the complex at 22° C. 1H NMR (400 MHz, C₆D₆) δ 8.55 (d, J=2.7 Hz, 1H), 8.16 (d, J=2.8 Hz, 1H), 4.31 (s, 3H), 3.60 (s, 3H), 1.89-0.90 (m, 60H). ¹³C{¹H} NMR (126 MHz, CDCl₃) δ 149.06, 148.84 (d, J_{C-P}=4.9 Hz), 133.65, 131.78 (dd, J_{C-P}=90.9, 39.8 Hz), 128.36, 117.12, 83.02, 57.06, 37.45, 35.44, 35.37, 35.30, 35.17, 34.91, 34.74, 34.59, 34.43, 31.65, 31.28, 30.45, 30.23, 30.20, 29.38, 29.20, 28.92, 28.89, 28.79, 28.51, 28.47, 28.38, 27.99, 27.91, 27.72, 27.64, 27.61, 27.59, 27.55, 27.51, 27.32, 27.27, 27.20, 27.07, 26.93, 26.84, 26.80, 26.73, 26.44, 26.28, 26.21, 26.16, 26.13, 26.04, 24.26, 23.65, 23.48, 23.31, 19.45, 19.35, 19.29, 19.20. A signal for C¹—B was not observed. ³¹P{¹H} NMR (162 MHz, C₆D₆) δ 64.14 (d, Jₚₚ=27.4 Hz), 60.74 (d, Jₚₚ=27.4 Hz). HRMS (ESI⁺) 767.4152 calculated for C₄₁H₆₈BBrN₂NiO₃P₂[M−Br]⁺, found 767.4119.

The crystal data for complex 53 is summarized below. The ORTEP drawing is depicted in FIG. 6.

The structure is the one suggested. Disorder of DCM was modeled by selection of strongest Q peaks and separation into two parts. The reflection corresponding to hkl value 120 was omitted from the final refinement due to systematic error from the beamstop.

TABLE 3
Crystal data and structural refinement for complex 53.

| Empirical formula | C₄₃ H₇₂ B Br Cl₄ N₂ Ni O₃ P₂ | |
|---|---|---|
| Formula weight | 1018.19 | |
| Temperature | 130(2) K | |
| Wavelength | 0.71073 Å | |
| Crystal system | Monoclinic | |
| Space group | P2₁/c | |
| Unit cell dimensions | a = 13.1557(5) Å | a = 90° |
| | b = 23.0504(6) Å | b = 106.3910(10) ° |
| | c = 16.5409(5) Å | g = 90° |

| | |
|---|---|
| Volume | 4812.1(3) Å³ |
| Z | 4 |
| Density (calculated) | 1.405 Mg/m³ |
| Absorption coefficient | 1.561 mm⁻¹ |
| F(000) | 2136 |
| Crystal color, morphology | yellow, block |
| Crystal size | 0.070 × 0.070 × 0.070 mm³ |
| Theta range for data collection | 1.965 to 30.526° |
| Index ranges | −18 ≤ h ≤ 18, −32 ≤ k ≤ 31, −23 l ≤ 21 |
| Reflections collected | 53952 |
| Independent reflections | 14687 [R(int) = 0.0296] |
| Observed reflections | 11900 |
| Completeness to theta = 25.242∞ | 99.9% |
| Absorption correction | Semi-empirical from equivalents |
| Refinement method | Full-matrix least-squares on F² |
| Data / restraints / parameters | 14687 / 13 / 539 |
| Goodness-of-fit on F² | 1.021 |
| Final R indices [I > 2sigma(I)] | R1 = 0.0329, wR2 = 0.0699 |
| R indices (all data) | R1 = 0.0484, wR2 = 0.0758 |
| Extinction coefficient | n/a |
| Largest diff. peak and hole | 1.294 and −1.098 e · Å−3 |

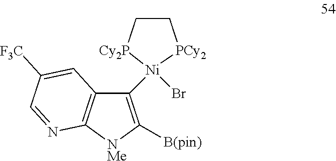

The general procedure was followed on 0.41 mmol scale of complex 51 to give complex 54 as an orange-yellow powder in 86% yield (310 mg). Single crystals suitable for X-ray crystallography were grown from a dichloromethane solution of the complex layered with pentane at 22° C. 1H NMR (400 MHz, C₆D₆) δ 8.94 (d, J=2.3 Hz, 1H), 8.89 (d, J=2.2 Hz, 1H), 4.18 (s, 3H), 1.96-0.93 (m, 60H). ¹³C{¹H} NMR (126 MHz, CDCl₃) δ 153.91 (d, J_{C-P}=2.7 Hz), 140.29, 136.98 (dd, J_{C-P}=91.4, 37.4 Hz), 130.02, 127.45, 125.93 (q, J_{C-F}=270.00 Hz), 115.49 (q, J_{C-F}=32.3 Hz), 83.36, 35.45, 35.27, 34.94, 34.80, 34.65, 31.60, 31.45, 30.48, 30.29, 29.24, 29.02, 28.75, 28.37, 27.94, 27.85, 27.65, 27.54, 27.25, 27.17, 27.04, 26.91, 26.82, 26.71, 26.39, 26.26, 26.12, 24.25, 23.61, 23.43, 23.25, 19.35. A signal for C¹—B was not observed. ³¹P{¹H} (162 MHz, C₆D₆) δ 65.46 (d, J_{PP}=29.7 Hz), 62.53 (d, Jₚₚ=29.7 Hz). ¹⁹F{¹H} NMR (376 MHz, C₆D₆) δ −58.45. HRMS (ESI⁺) 805.3920 calculated for C₄₁H₆₅BBrF₃N₂NiO₂P₂[M−Br]⁺, found 805.3919.

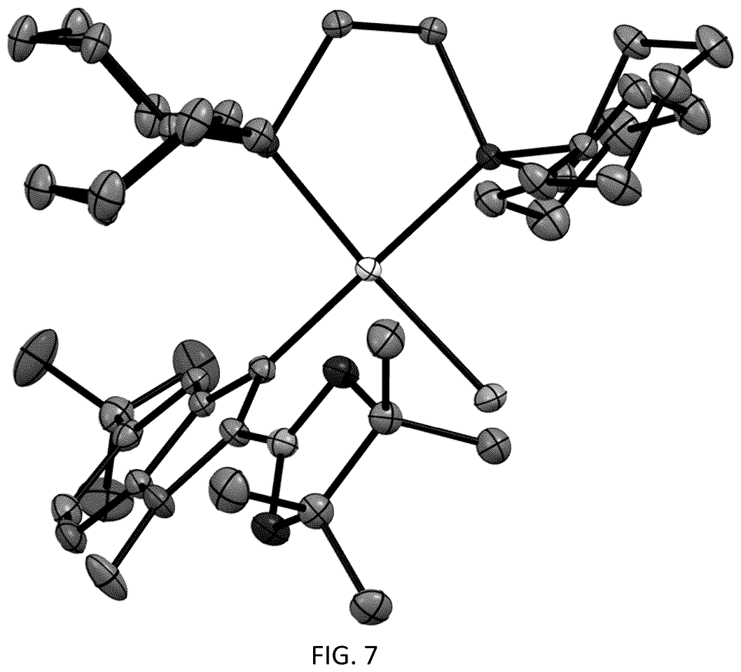

The crystal data for complex 54 is summarized below. The ORTEP drawing is depicted in FIG. 7.

The structure is the one suggested. Disorder of dichloromethane was modeled by selection of strongest Q peaks and separation into 2 parts.

TABLE 4
Crystal data and structural refinement for complex 54

| Empirical formula | C₄₂ H₆₇ B Br Cl₂ F₃ N₂ Ni O₂ P₂ | |
|---|---|---|
| Formula weight | 971.24 | |
| Temperature | 130(2) K | |
| Wavelength | 0.71073 Å | |
| Crystal system | Monoclinic | |
| Space group | P2₁/c | |
| Unit cell dimensions | a = 13.5413(7) Å | a = 90° |
| | b = 21.8075(11) Å | b = 93.568(2) ° |
| | c = 15.7587(6) Å | g = 90° |

| | |
|---|---|
| Volume | 4644.6(4) Å³ |
| Z | 4 |
| Density (calculated) | 1.389 Mg/m³ |
| Absorption coefficient | 1.510 mm⁻¹ |
| F(000) | 2032 |
| Crystal color, morphology | yellow, needle |
| Crystal size | 0.150 × 0.075 × 0.075 mm³ |
| Theta range for data collection | 2.139 to 30.536° |
| Index ranges | −19 ≤ h ≤ 19, −31 ≤ k ≤ 23, −15 ≤ l ≤ 22 |
| Reflections collected | 42233 |
| Independent reflections | 14199 [R(int) = 0.0330] |
| Observed reflections | 11067 |
| Completeness to theta = 25.242∞ | 99.9% |
| Absorption correction | Semi-empirical from equivalents |
| Refinement method | Full-matrix least-squares on F² |
| Data / restraints / parameters | 14199 / 7 / 520 |
| Goodness-of-fit on F2 | 1.020 |
| Final R indices [I > 2sigma(I)] | R1 = 0.0410, wR2 = 0.0974 |
| R indices (all data) | R1 = 0.0607, wR2 = 0.1066 |
| Extinction coefficient | n/a |
| Largest diff. peak and hole | 1.316 and -1.199 e · Å⁻³ |

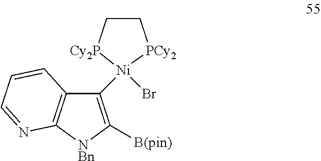

The general procedure was followed on 0.45 mmol complex 52 to give complex 55 in as an orange-yellow solid in 87% yield (352 mg). Single crystals suitable for X-ray crystallography were grown from a THF solution of the complex with slow vapor diffusion of pentane at 22° C. ¹H NMR (400 MHz, CDCl₃) δ 8.27 (d, J=4.6 Hz, 1H), 8.21 (d, J=7.7 Hz, 1H), 7.27 (s, 1H), 7.13 (td, J=17.8, 16.6, 7.6 Hz, 4H), 6.91-6.83 (m, 1H), 5.89 (d, J=15.3 Hz, 1H), 5.72 (d, J=15.3 Hz, 1H), 2.71 (m, 1H), 2.37 (d, J=12.0 Hz, 4H), 2.26-2.12 (m, 1H), 1.95-0.71 (m, 54H). ¹³C{¹H} NMR (126 MHz, CDCl₃) δ 152.70 (d, J_{C-P}=4.9 Hz), 143.38, 141.94, 135.73 (dd, J_{C-P}=90.9, 38.6 Hz), 133.15, 128.69, 127.79, 127.62, 126.13, 113.13, 83.07, 46.44, 35.60, 35.51, 35.45, 35.31, 34.88, 34.73, 34.70, 34.56, 31.00, 30.41, 30.02, 29.99, 29.35, 29.09, 29.07, 28.79, 28.75, 28.71, 27.71, 27.67, 27.61, 27.59, 27.56, 27.52, 27.50, 27.30, 27.25, 27.18, 27.14, 27.09, 27.04, 26.95, 26.89, 26.82, 26.41, 26.11, 26.05, 25.92, 25.17, 24.75, 24.14, 23.82, 23.64, 23.47, 19.98, 19.88, 19.82, 19.73. A signal for C¹—B was not observed. ³¹P{¹H} (162 MHz, CDCl₃) δ 65.09 (d, J_{PP}=28.3 Hz), 61.61 (d, Jₚₚ=28.3 Hz). HRMS (ESI⁺) 813.4359 calculated C₄₆H₇₀BBrN₂NiO₂P₂[M−Br]⁺, found 813.4363.

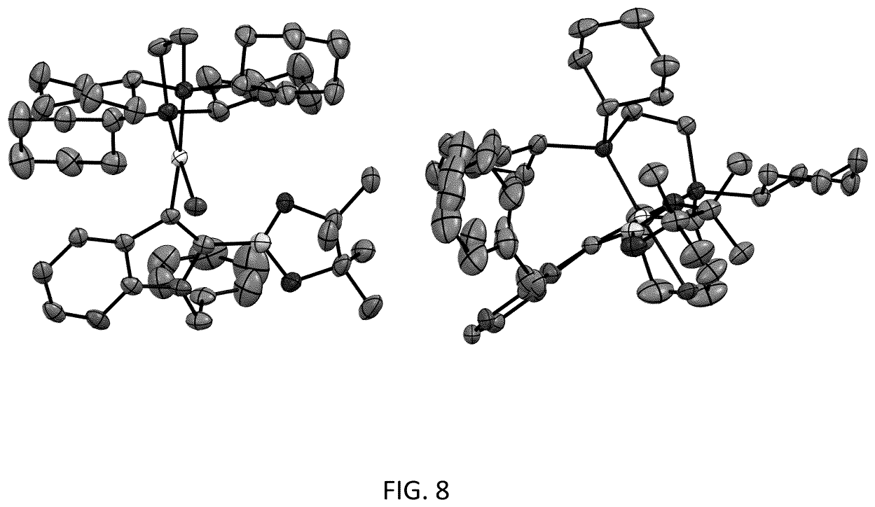

The crystal data for complex 55 is summarized below. The ORTEP drawing is depicted in FIG. 8.

This crystal structure has two formula units of the nickel complex in the asymmetric unit along with three zones of solvent of crystallization. Two of these solvent molecules were initially modeled as n-pentane while the third appeared to be an admixture of both n-pentane and tetrahydrofuran. The anisotropic displacement parameters were large compared to the nickel complexes. The addition of restraints did not benefit the model. Refinement of the occupancy of each returned values near unity. Given these poor solvent models PLATON/SQUEEZE (62) was used to determine the solvent void volume (2307 Å, 21.5%) and electron density (499 electrons) within. These results corroborate nearly fully-occupied solvent positions. The solvent-corrected data by PLATON/SQUEEZE improved the R1>2%. Following this, the benzyl disorder for the B-molecule was successfully completed.

TABLE 5
Crystal data and structural refinement for complex 55

| Empirical formula | C₄₆ H₇₀ B Br N₂ Ni O₂ P₂ | |
|---|---|---|
| Formula weight | 894.41 | |
| Temperature | 130(2) K | |
| Wavelength | 0.71073 Å | |
| Crystal system | Monoclinic | |
| Space group | Cc | |
| Unit cell dimensions | a = 15.4458(8) Å | a = 90° |
| | b = 28.7404(12) Å | b = 90.336(2) ° |
| | c = 24.1301(9) Å | g = 90° |

| | |
|---|---|
| Volume | 10711.6(8) Å |
| Z | 8 |
| Density (calculated) | 1.109 Mg/m³ |
| Absorption coefficient | 1.200 mm⁻¹ |
| F(000) | 3792 |
| Crystal color, morphology | yellow, block |
| Crystal size | 0.080 × 0.080 × 0.060 mm³ |
| Theta range for data collection | 2.204 to 26.398° |
| Index ranges | −19 ≤ h ≤ 19, −35 ≤ k ≤ 35, −30 ≤ l ≤ 30 |
| Reflections collected | 186390 |
| Independent reflections | 21972 [R(int) = 0.0573] |
| Observed reflections | 20025 |
| Completeness to theta = 25.242° | 99.9% |
| Absorption correction | Semi-empirical from equivalents |
| Max. and min. transmission | 0.5624 and 0.5303 |
| Refinement method | Full-matrix least-squares on F² |
| Data / restraints / parameters | 21972 / 365 / 1057 |
| Goodness-of-fit on F² | 1.019 |
| Final R indices [/>2sigma(I)] | R1 = 0.0292, wR2 = 0.0634 |
| R indices (all data) | R1 = 0.0362, wR2 = 0.0663 |
| Absolute structure parameter | 0.0005(18) |
| Extinction coefficient | n/a |
| Largest diff. peak and hole | 0.496 and −0.230 e · Å³ |

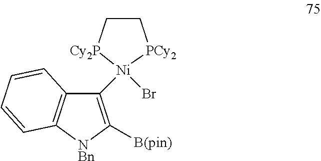

The general procedure was followed to give complex 75 in 84% yield. ¹H NMR (500 MHz, CDCl₃) δ 8.00 (d, J=7.9 Hz, 1H), 7.33 (s, 0H), 7.18 (d, J=7.1 Hz, 5H), 7.12 (d, J=6.6 Hz, 1H), 7.06 (t, J=7.5 Hz, 1H), 6.91 (t, J=7.4 Hz, 1H), 6.00 (d, J=16.0 Hz, 1H), 5.30 (d, J=16.0 Hz, 1H), 2.75 (t, J=8.3 Hz, 1H), 2.66 (d, J=12.5 Hz, OH), 2.39 (t, J=10.4 Hz, 3H), 2.21 (q, J=11.7 Hz, 1H), 1.94-1.40 (m, 41H), 1.31 (d, J=6.4 Hz, 17H), 1.26-0.76 (m, overlapping with B(pin) methyl signal, 9H). ¹³C{¹H} NMR (126 MHz, CDCl₃) δ 141.90, 141.73, 136.90, 127.98, 127.15, 126.24, 126.11, 122.04, 116.79, 109.44, 82.80, 48.59, 35.59-35.24 (m), 34.75 (t, J_{CP}=24.4 Hz), 31.09, 30.04, 29.14, 28.78, 27.73, 27.40-26.98 (m, overlapping multiplets), 26.78 (d, J_{CP}=8.7 Hz), 26.47, 26.33-25.99 (m, overlapping multiplets), 24.10, 23.65 (t, J_{CP}=22.5 Hz), 19.80 (dd, J_{CP}=19.3 Hz, 11.4 Hz). ³¹P{¹H} (162 MHz, CDCl₃): δ 63.92 (d, J_{PP}=27.2 Hz), 60.68 (d, J_{PP}=27.2 Hz). HRMS (ESI⁺) 812.4407 calculated for C₄₇H₇₁BBrNNiO₂P₂ [M−Br]⁺, found 812.4427.

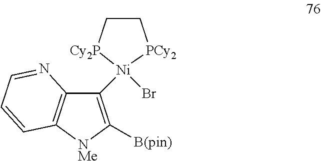

The general procedure was followed to give complex 76 in 64% yield. ¹H NMR (400 MHz, CDCl₃) 8.34 (d, J=4.4 Hz, 1H), 7.36 (d, J=8.0 Hz, 1H), 6.92 (dd, J=8.2, 4.4 Hz, 1H), 3.90 (s, 3H), 3.75-3.65 (m, 1H), 2.74-2.62 (m, 2H), 2.24-0.80 (m, overlapping with B(pin) methyl signal, 53H), 1.42 (s, 6H), 1.38 (s, 6H), 0.52-0.39 (m, 1H). ¹³C NMR (126 MHz, CDCl₃) 154.75, 141.07, 135.10 (dd, J=88.9, 40.9 Hz), 134.05, 134.02, 115.86, 115.30, 82.98, 35.98, 35.87, 35.67, 34.61 (d, J=21.2 Hz), 33.95 (d, J=20.5 Hz), 32.98, 32.48, 30.47, 29.75, 29.40, 29.06, 28.90, 28.43, 27.85, 27.76, 27.55, 27.41, 27.34, 27.07, 26.57, 26.12, 24.38, 23.76, 23.59, 23.41, 19.89 (dd, J=19.7, 12.2 Hz). ³¹P{¹H} (162 MHz, CDCl₃): 67.46 (d, Jₚₚ=30.0 Hz), 59.99 (d, Jₚₚ=30.3 Hz). HRMS (ESI⁺) 737.4046 calculated for C₄₀H₆₆BBrN2NiO₂P₂[M−Br]⁺, found 737.4079.

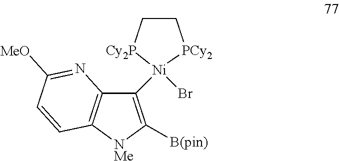

The general procedure was followed to give complex 77 in 68% yield. Single crystals suitable for X-ray crystallography were grown from a DCM solution of the complex with slow vapor diffusion of pentane at 22° C. 1H NMR (500 MHz, CDCl₃) 7.31 (d, J=8.7 Hz, 1H), 6.47 (d, J=8.6 Hz, 1H), 4.00 (s, 3H), 3.86 (s, 3H), 3.06-2.96 (m, 1H), 2.68-2.50 (m, 2H), 2.40-2.21 (m, 3H), 2.20-2.05 (m, 1H), 2.01-1.45 (overlapping peaks, 27H), 1.43-1.10 (m, overlapping with B(pin) methyl signal 14H), 1.38 (s, 6H), 1.34 (s, 6H), 1.05-0.78 (m, 4H), 0.76-0.60 (m, 1H). ¹³C NMR (126 MHz, CDCl₃) 157.40, 150.47, 134.20 (d, J_{CP}=39.8 Hz), 133.50 (d, J_{CP}=39.6 Hz), 131.11, 119.24, 104.94, 82.87, 52.84, 35.68 (d, J_{CP}=20.6 Hz), 35.27 (d, J_{CP}=23.8 Hz), 34.83 (d, J_{CP}=4.6 Hz), 34.66, 32.85, 30.40, 30.07, 29.68, 29.26, 28.95, 28.34, 28.22, 28.13, 27.76, 27.69, 27.60, 27.51, 27.39, 27.33, 27.25, 27.18, 27.05, 26.97, 26.73, 26.66, 26.48, 26.41, 26.36, 26.12, 26.04, 24.14, 23.92, 23.74, 23.57, 19.36 (dd, J_{CP}=19.0, 11.9 Hz). ³¹P{¹H}(162 MHz, CDCl₃): 65.30 (d, J_{PP}=30.6 Hz), 63.37 (d, J_{PP}=31.1 Hz). HRMS (ESI⁺) 767.4152 calculated for C₄₁H₆₈BBrN2NiO₃P₂[M−Br]⁺, found 767.4178.

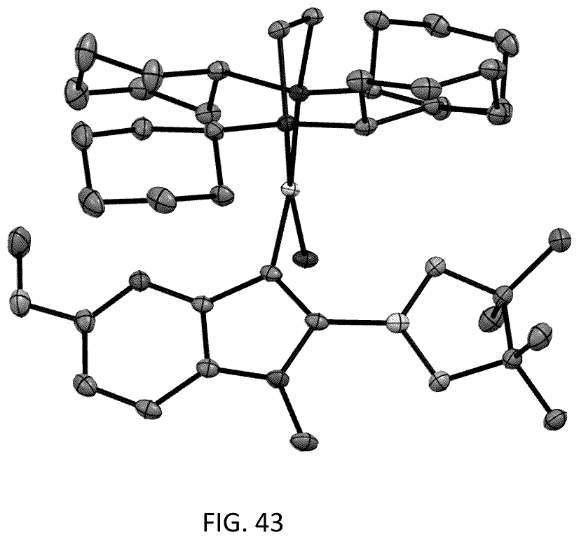

The crystal data for complex 77 is summarized below. The ORTEP drawing is depicted in FIG. 43.

TABLE 6
Crystal data and structural refinement for complex 77.

| Empirical formula | C₄₃ H₇₂ B Br Cl₄ N₂ Ni O₃ P₂ | |
|---|---|---|
| Formula weight | 1018.19 | |
| Temperature | 100(2) K | |
| Wavelength | 0.71073 Å | |
| Crystal system | Monoclinic | |
| Space group | P2₁/c | |
| Unit cell dimensions | a = 13.3289(6) Å | a = 90° |
| | b = 23.2105(11) A | b = 107.0490(10) ° |
| | c = 16.3200(7) Å | g = 90° |

| | |
|---|---|
| Volume | 4827.0(4) Å^{a} |
| Z | 4 |
| Density (calculated) | 1.401 Mg/m³ |
| Absorption coefficient | 1.557 mm⁻¹ |
| F(000) | 2136 |
| Crystal color, morphology | yellow, block |
| Crystal size | 0.070 × 0.070 × 0.070 mm³ |
| Theta range for data collection | 1.950 to 28.366° |
| Index ranges | −17 £ h £ 17, −30 £ k £ 29, −17 £ l £ 21 |
| Reflections collected | 56039 |
| Independent reflections | 12054 [R(int) = 0.0458] |
| Observed reflections | 9996 |
| Completeness to theta = 25.242∞ | 100.0% |
| Absorption correction | Semi-empirical from equivalents |
| Max. and min. transmission | 0.7457 and 0.689 |
| Refinement method | Full-matrix least-squares on F² |
| Data / restraints / parameters | 12054 / 39 / 546 |
| Goodness-of-fit on F2 | 1.030 |
| Final R indices [I > 2sigma(I)] | R1 = 0.0332, wR2 = 0.0773 |
| R indices (all data) | R1 = 0.0456, wR2 = 0.0826 |
| Extinction coefficient | n/a |
| Largest diff. peak and hole | 0.619 and −0.710 eÅ⁻³ |

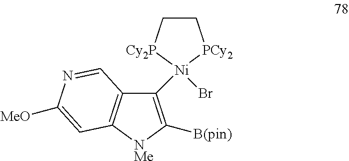

The general procedure was followed to give complex 78 in 85% yield. ¹H NMR (400 MHz, CDCl₃): δ 8.81 (s, 1H), 6.34 (s, 1H), 3.95 (s, 3H), 3.75 (d, J=12.6 Hz, 3H), 2.60-1.98 (m, 7H), 1.93-1.48 (m, 34H), 1.41-0.77 (m, overlapping with B(pin) methyl signal, 38H). ¹³C{¹H} NMR (101 MHz, CDCl₃) δ 160.43, 148.86, 144.71, 131.14, 129.03, 128.22, 125.29, 86.51, 82.81, 54.00, 37.33, 35.87-34.17 (m, overlapping multiplets), 32.28, 31.49, 30.32, 29.91, 29.45-26.48 (m, overlapping multiplets), 26.12, 24.08, 23.39 (t, J_{CP}=22.1 Hz), 19.80-19.27 (m, overlapping multiplets). ³¹P{¹H} NMR (162 MHz, CDCl₃) δ 64.49 (d, J_{PP}=29.8 Hz), 60.59 (d, J_{PP}=29.5 Hz). HRMS (ESI⁺): 767.4146 calculated for C₄₁H₆₈BBrN2NiO₃P₂[M−Br]⁺, found 767.4175.

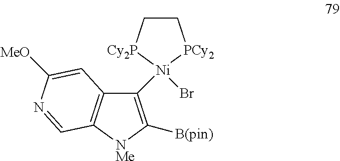

The general procedure was followed to give complex 79 in 86% yield. ¹H NMR (500 MHz, CDCl₃) δ 8.18 (s, 1H), 7.18 (s, 1H), 3.95 (s, 3H), 3.93 (s, 3H), 2.70 (d, J=39.6 Hz, 3H), 2.34 (d, J=13.2 Hz, 4H), 2.21 (t, J=11.5 Hz, 1H), 2.06-0.75 (m, overlapping with B(pin) methyl signal, 86H). ¹³C{¹H} NMR (126 MHz, CDCl₃) δ 156.56, 144.41, 136.29, 131.39 (dd, J_{CP}=40.1 Hz, 38.5), 129.10, 128.29, 125.37, 101.90, 83.23, 54.24, 37.40 (t, J_{CP}=13.0 Hz), 35.43 (d, J_{CP}=25.9 Hz), 34.67 (d, J_{CP}=21.2 Hz), 34.25 (d, J_{CP}=20.9 Hz), 32.71, 32.29, 30.39, 30.04, 29.33, 29.06, 28.70, 28.40, 27.91-27.45 (m, overlapping multiplets), 27.38-26.96 (m, overlapping multiplets), 26.14, 24.27, 23.41 (t, J_{CP}=21.8 Hz), 22.50, 19.48 (dd, J_{CP}=19.4, 11.5 Hz). ³¹P{¹H} (203 MHz, CDCl₃): δ 64.54 (d, Jₚₚ=29.2 Hz), 60.06 (d, Jₚₚ=28.9 Hz). HRMS (ESI⁺) 767.4152 calculated for C₄₁H₆₈BBrN2NiO₃P₂[M−Br]⁺, found 767.4182.

General Procedure for Transmetallation of DCPE α-aryl Complexes

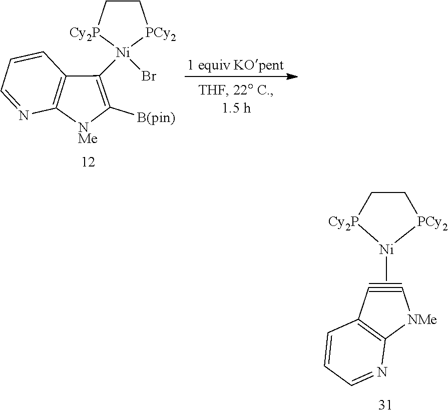

Synthesis of DCPE-Ni-7-azaindolyne 31

12 (250 mg, 0.306 mmol, 1 equiv) was dissolved in THF (9 mL, 0.03 M). A solution of potassium tert-pentoxide (180 μL, 1.7 M in PhMe, 1.0 equiv) was added. The solution immediately underwent a color change from yellow to red/orange. The reaction was allowed to stir for 1.5 h, then pentane (3 mL) was added. The solution was filtered through celite and the celite was washed with a 1:1 THF:pentane solution (5 mL). The solvent was removed from the filtrate under vacuum to afford a dark red residue which precipitated an orange solid upon trituration with cold pentane (3×2 mL) to yield complex 31 in 96% yield (187 mg). Single crystals suitable for X-Ray crystallography were obtained from a solution of the complex in THF with slow vapor diffusion of pentane at 22° C.

### Procedure for Scale Up of Transmetallatation of DCPE σ-Aryl Complex

12 (700 mg, 0.855 mmol, 1 equiv) was dissolved in THF (57 mL, 0.015 M). A solution of potassium tert-pentoxide (604 μL, 1.7 M in PhMe, 1.2 equiv) was added. The solution immediately underwent a color change from yellow to red/orange. The reaction was allowed to stir for 1.5 h, then pentane (30 mL) was added. The solution was filtered through celite and the celite was washed with a 1:1 THF:pentane solution (5 mL). The solution was portioned into two vials and the solvent was removed from the filtrate under vacuum to afford a dark brown residue. Upon the addition of a few drops of THF, an orange solid was crashed out and trituration with cold pentane (5×2 mL) yielded complex 31 in 91% yield (474 mg).

¹H NMR (500 MHz, THF-d₈): 7.88 (d, J=4.8 Hz, 1H), 7.58 (d, J=7.6 Hz, 1H), 6.71 (dd, J=7.6, 4.8 Hz, 1H), 3.87 (s, 3H), 2.30-0.70 (overlapping peaks, 48H). ¹³C{¹H} NMR (126 MHz, THF-d₈): 166.91 (dd, J_{PC}=91, 11 Hz), 165.23, 139.63, 128.22, 121.25 (dd, J_{PC}=83, 17 Hz), 120.51 (dd, J_{PC}=10, 7 Hz), 114.23, 36.07 (dd, J_{PC}=29, 21 Hz), 33.73, 30.72 (dd, J_{PC}=42, 4 Hz), 30.28 (d, J_{PC}=23 Hz), 28.19 (m), 27.28, 27.25, 2289 (m). ³¹P{¹H} NMR (203 MHz, THF-d₈): 90.14 (d, J_{PP}=4.5 Hz), 85.65 (d, J_{PP}=4.8 Hz). HRMS (ESI⁺) 611.3189 calculated for C₃₄H₅₄N₂NiP₂ [M+H]⁺, found 611.3210. Note: Peaks in the alkyl region of ¹H NMR spectra often over-integrate due to THF-d₈ signal.

Note: Interestingly, in complexes of dcpe Ni 6-membered arynes with unsymmetric substitution (m-Me, m-CO₂Me, m-CF₃), the ³¹P{¹H} NMR spectra generally show one singlet, indicating both phosphines are in a nearly identical chemical environment, despite the benzyne substitution. The only case where the phosphine resonances separate out is with a m-Ome substituent, but this only separates the singlets by 0.6 ppm and no coupling constant is reported. This is in contrast to the 5-membered heteroaryne complexes which all appear as two distinct doublets, indicating the two P donors are chemically inequivalent.

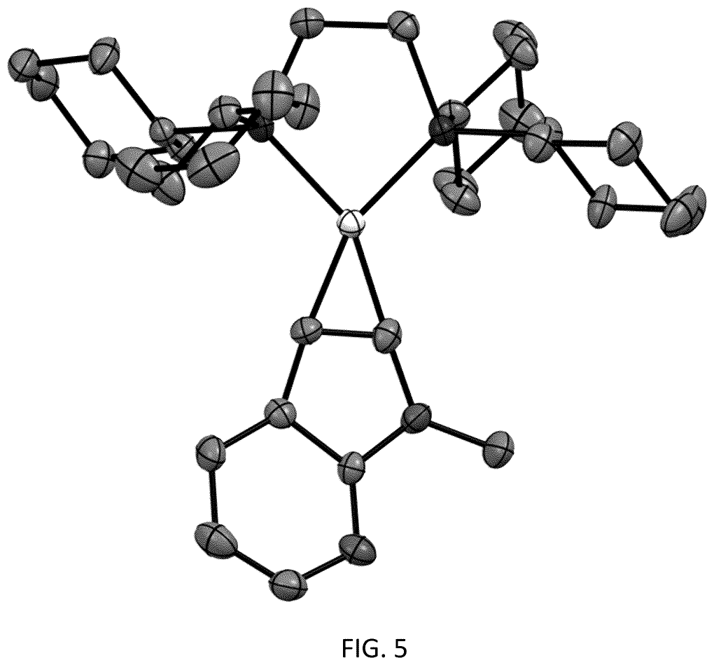

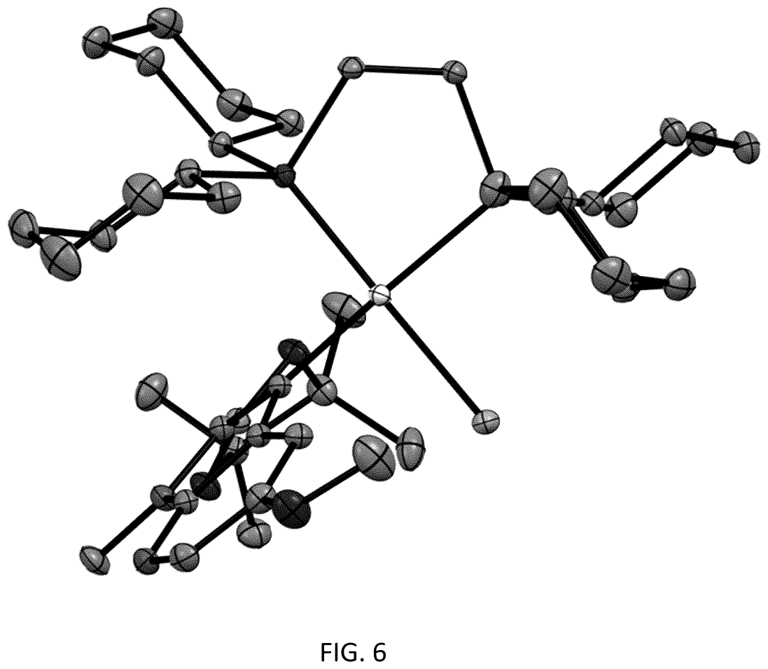

The crystal data for complex 31 is summarized below. The ORTEP drawing is depicted in FIG. 5.

The structure is the one suggested. The reflection corresponding to hkl value 101 was omitted from the final refinement due to systematic error from the beamstop.

TABLE 7
Crystal data and structural refinement for complex 31.

| Empirical formula | C₃₄ H₅₄ N₂ Ni P₂ | |
|---|---|---|
| Formula weight | 611.44 | |
| Temperature | 130(2) K | |
| Wavelength | 0.71073 Å | |
| Crystal system | Orthorhombic | |
| Space group | P2₁2₁2₁ | |
| Unit cell dimensions | a = 10.7269(7) Å | α = 90° |
| | b = 16.5580(12) Å | β = 90° |
| | c = 18.5025(14) Å | γ = 90° |

| | |
|---|---|
| Volume | 3286.3(4) Å³ |
| Z | 4 |
| Density (calculated) | 1.236 Mg/m³ |
| Absorption coefficient | 0.713 mm⁻¹ |
| F(000) | 1320 |
| Crystal color, morphology | yellow, plate |
| Crystal size | 0.200 × 0.200 × 0.200 mm³ |
| Theta range for data collection | 2.201 to 25.725° |
| Index ranges | −13 ≤ h ≤ 12, −18 ≤ k ≤ 20, −22 ≤ l ≤ 20 |
| Reflections collected | 23602 |
| Independent reflections | 6237 [R(int) = 0.0661] |
| Observed reflections | 5377 |
| Completeness to theta = 25.242° | 99.9% |
| Absorption correction | Multi-scan |
| Refinement method | Full-matrix least-squares on F² |
| Data / restraints / parameters | 6237 / 0 / 353 |
| Goodness-of-fit on F² | 1.081 |
| Final R indices [I > 2sigma(I)] | R1 = 0.0507, wR2 = 0.1152 |
| R indices (all data) | R1 = 0.0641, wR2 = 0.1223 |
| Extinction coefficient | n/a |
| Largest diff. peak and hole | 0.868 d −0.304 e · Å⁻³ |

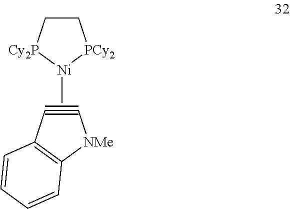

The general procedure was followed to give DCPE-Ni-7-indolyne 32 in 80% yield. Single crystals suitable for X-ray diffraction analysis were obtained from a THF solution layered with pentane. ¹H NMR (C₆D₆): 8.10 (d, ³J_{HH} 7.3 Hz, 1H), 7.37 (m, 2H), 7.21 (d, ³J_{HH} 8.2 Hz, 1H), 3.79 (s, 3H), 3.26 (q, ³J_{HH} 6.9 Hz, 1H), 2.20 (dd, ³J_{HH} 32.8, 12.4 Hz, 4H), 2.00-0.80 (overlapping peaks, 43H). ³¹P{¹H} NMR (C₆D₆): 90.7 (d, ²J_{PP} 5.0 Hz), 84.8 (d, ²J_{PP} 5.1 Hz). ¹³C{¹H} NMR (C₆D₆): 167.3 (dd, ²J_{PC} 92, 10 Hz), 157.2 (s), 126.1 (dd, ³J_{PC} 9 Hz), 124.9 (dd, ²J_{PC} 83, 17 Hz), 122.8 (s), 119.8 (s), 117.7 (s), 110.2 (s), 65.6 (s), 35.2 (d, 21 Hz), 34.9 (d, 21 Hz), 35.2 (s), 29.8 (d, 4 Hz), 29.5 (d, 3 Hz), 29.2 (s), 29.1 (s), 27.2 (s), 27.1 (s), 27.0 (s), 26.9 (s), 26.2 (s), 26.1 (s), 22.1 (m). HRMS (ESI⁺) 610.3238 calculated for C₃₅H₅₅NNiP₂ [M+H]⁺, found 610.3248.

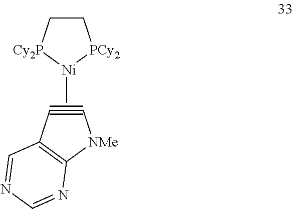

The general procedure was followed to give complex 33 in 70% yield. ¹H NMR (THF-d₈): δ 8.54 (s, 1H), 8.46 (s, OH), 3.85 (s, 3H), 2.08-1.02 (m, 53H). ³¹P{¹H} NMR (THF-d₈) δ 89.91 (d, J=2.1 Hz), 85.92 (d, J=2.1 Hz). ¹³C{¹H} NMR (THF-d₈): δ 168.93 (dd, J_{PC}=91.2, 10.7 Hz), 167.06, 149.75, 148.26, 129.20, 120.46 (dd, J_{PC}=83.4, 17.2 Hz), 119.01-118.56 (overlapping multiplet), 36.03 (dd, J_{PC}=34.5, 21.4 Hz), 33.37, 30.90 (d, J_{PC}=3.7 Hz), 30.58 (d, J_{PC}=3.1 Hz), 30.30 (d, J_{PC}=21.2 Hz), 28.39-27.78 (m), 27.24, 26.03-25.22 (m), 23.21-22.43 (m). HRMS (ESI⁺) 612.3141 calculated for C₃₃H₅₃N₃NiP₂, found 612.3141.

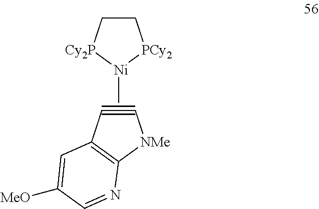

The general procedure was followed to give complex 56 in >98% yield (189 mg). Single crystals suitable for X-ray crystallography were grown from a THF solution of the complex with slow vapor diffusion of pentane at 22° C. ¹H NMR (500 MHz, THF-d₈): δ 7.68 (d, J=2.7 Hz, 1H), 7.19 (d, J=2.9 Hz, 1H), 3.83 (s, 4H), 3.78 (s, 4H), 2.23-0.80 (overlapping peaks, 48H). ¹³C{¹H} (126 MHz, THF-d₈): δ 168.25 (dd, J_{PC}=90.4, 10.4 Hz), 161.53, 151.80, 128.22, 119.73 (dd, J_{PC}=83.5, 16.5 Hz), 119.97 (dd, J_{PC}=10 Hz), 114.07, 57.11, 36.07 (t, J_{PC}=21.3 Hz), 33.82, 30.74 (dd, J_{PC}=37.5, 3.9 Hz), 30.27 (d, J_{PC}=23.9 Hz), 28.48-27.90 (m), 27.28, 25.57 (dq, J_{PC}=40.0, 20.5 Hz), 22.86 (dt, J_{PC}=23.7, 16.7 Hz). ³¹P{¹H} NMR (203 MHz, THF-d₈): δ 90.02 (d, J_{PP}=4.5 Hz), 85.44 (d, J_{PP}=4.9 Hz). HRMS (ESI⁺) 640.3294 calculated for C₃₅H₅₆N₂NiOP₂ [M+H]⁺, found 640.3290.

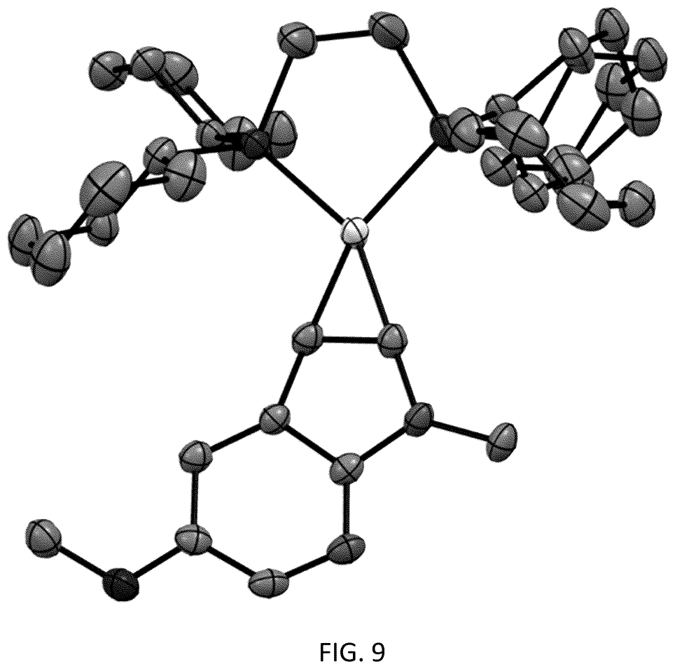

The crystal data for complex 56 is summarized below. The ORTEP drawing is depicted in FIG. 9.

The structure is the one suggested.

TABLE 8
Crystal data and structural refinement for complex 56.

| Empirical formula | C₃₅ H₅₆ N₂ Ni O P₂ | |
|---|---|---|
| Formula weight | 641.46 | |
| Temperature | 130(2) K | |
| Wavelength | 0.71073 Å | |
| Crystal system | Monoclinic | |
| Space group | P2₁/n | |
| Unit cell dimensions | a = 14.7069(6) Å | a = 90° |
| | b = 14.7263(8) Å | b = 101.754(2) ° |
| | c = 15.9514(7) Å | g = 90° |

| | |
|---|---|
| Volume | 3382.3(3) Å³ |
| Z | 4 |
| Density (calculated) | 1.260 Mg/m³ |
| Absorption coefficient | 0.698 mm⁻¹ |
| F(000) | 1384 |
| Crystal color, morphology | yellow, plate |
| Crystal size | 0.150 × 0.100 × 0.050 mm³ |
| Theta range for data collection | 1.901 to 26.391° |
| Index ranges | −15 ≤ h ≤ 18, −18 ≤ k ≤ 18, −19 ≤ l ≤19 |
| Reflections collected | 36286 |
| Independent reflections | 6926 [R(int) = 0.0444] |
| Observed reflections | 5172 |
| Completeness to theta = 25.242° | 100.0% |
| Absorption correction | Semi-empirical from equivalents |
| Refinement method | Full-matrix least-squares on F² |
| Data / restraints / parameters | 6926 / 13 / 391 |
| Goodness-of-fit on F² | 1.048 |
| Final R indices [I > 2sigma(I)] | R1 = 0.0410, wR2 = 0.0907 |
| R indices (all data) | R1 = 0.0669, wR2 = 0.1065 |
| Extinction coefficient | n/a |
| Largest diff. peak and hole | 0.868 0.618 and −0.523 e · Å⁻³ |

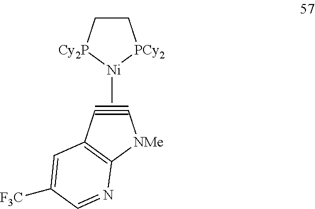

The general procedure was followed on 0.362 mmol scale of complex 54 to give complex 57 in >98% yield (244 mg). Several attempts to obtain crystal suitable for X-ray crystallography were unsuccessful or lead to decomposition. ¹H NMR (500 MHz, THF) δ 8.21 (s, 1H), 7.80 (s, 1H), 3.92 (s, 3H), 2.25-1.04 (overlapping peaks, 48H). ¹³C{¹H} NMR (THF) δ 170.58 (dd, J_{PC}=90.1, 10.7 Hz), 165.89, 136.43 (q, J_{FC}=4.6 Hz), 127.71 (q, J_{PC}=270.7 Hz), 124.63 (q, J_{PC}=3.7 Hz), 120.49 (dd, J_{PC}=83.4, 16.9 Hz), 119.32 (q, J_{FC}=9.1 Hz), 117.16 (q, J_{FC}=30.6 Hz), 36.01 (dd, J_{PC}=21.4, 17.7 Hz), 33.92, 30.92 (d, J_{PC}=3.9 Hz), 30.30 (d, J_{PC}=26.2 Hz), 28.36-27.94 (m), 27.24, 25.73 (d, J_{PC}=20.1). ³¹P{¹H} NMR (162 MHz, THF) δ 89.58 (d, J=2.1 Hz), 86.01 (d, J=2.1 Hz). ¹⁹F{¹H} NMR (471 MHz, THF) δ −59.55. HRMS (ESI⁺) 679.3062 calculated for C₃₅H₅₃F₃N₂NiP₂ [M+H]⁺, found 679.3039.

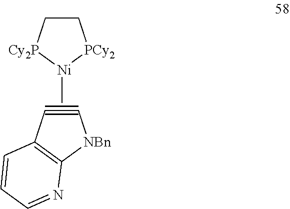

The general procedure was followed on 0.280 mmol scale of complex 55 to give complex 58 in 91% yield (175 mg). Single crystals suitable for X-ray crystallography were grown from a THF solution of the complex with slow vapor diffusion of pentane at 22° C. ¹H NMR (500 MHz, THF) δ 7.88 (d, J=4.7 Hz, 1H), 7.61 (d, J=7.6 Hz, 1H), 7.27 (d, J=7.6 Hz, 2H), 7.17 (t, J=7.5 Hz, 2H), 7.10 (t, J=7.5 Hz, 1H), 6.76 (dd, J=6.3, 5.0 Hz, 1H), 5.51 (s, 2H), 2.21-1.02 (overlapping peaks, 48H). ¹³C{¹H} NMR (126 MHz, THF) δ 166.00 (dd, J_{PC}=91.6, 10.8 Hz), 165.28, 142.46, 139.74, 128.75, 128.72, 128.37, 127.07, 122.47 (dd, J_{PC}=82.5, 16.7 Hz), 120.40 (dd, J_{PC}=9.7, 3.1 Hz), 114.70, 50.59, 36.00 (dd, J_{PC}=58.7, 21.0 Hz), 30.78 (d, J_{PC}=4.2 Hz), 30.58 (d, J_{PC}=3.4 Hz), 30.22 (d, J_{PC}=3.6 Hz), 28.30-27.94 (m), 27.22 (d, J_{PC}=6.5 Hz), 25.57 (dq, J_{PC}=40.1, 20.6 Hz), 22.79 (ddd, J_{PC}=75.4, 23.3, 17.2 Hz). ³¹P{¹H} NMR (203 MHz, THF) δ 90.03 (d, Jₚₚ=4.0 Hz), 85.07 (d, Jₚₚ=3.9 Hz). HRMS (ESI⁺) 687.3501 calculated for C₄₁H₆₁N₂P₂ [M+H]⁺, found 687.3527.

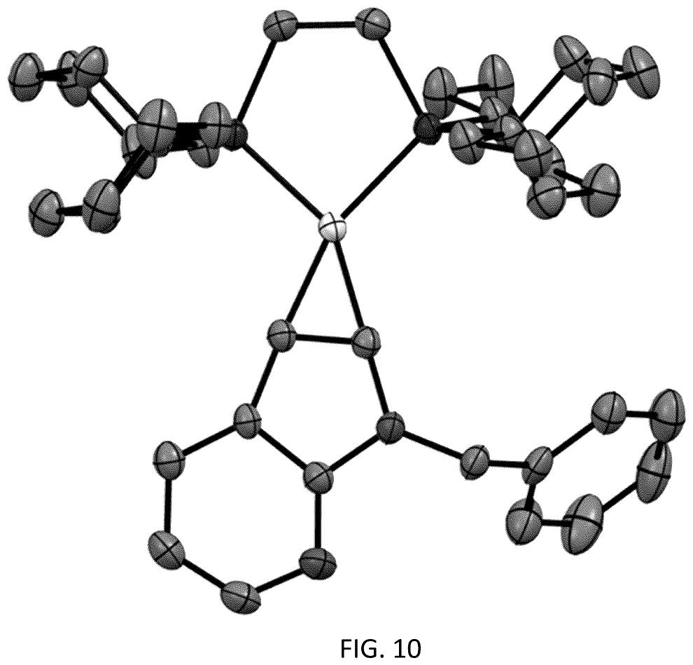

The crystal data for complex 58 is summarized below. The ORTEP drawing is depicted in FIG. 10.

The structure is the one suggested. The reflection corresponding to hkl value 002 was omitted from the final refinement due to systematic error from the beamstop.

TABLE 9
Crystal data and structural refinement for complex 58.

| Empirical formula | C₄₀ H₅₈ N₂ Ni P₂ | |
|---|---|---|
| Formula weight | 687.53 | |
| Temperature | 130(2) K | |
| Wavelength | 0.71073 Å | |
| Crystal system | Monoclinic | |
| Space group | P2₁/c | |
| Unit cell dimensions | a = 11.7631(3) Å | α = 90° |
| | b = 15.7233(3) Å | β = 101.8500(10) ° |
| | c = 20.2760(5) Å | γ = 90° |

| | |
|---|---|
| Volume | 3670.22(15) Å³ |
| Z | 4 |
| Density (calculated) | 1.244 Mg/m³ |
| Absorption coefficient | 0.646 mm⁻¹ |
| F(000) | 1480 |
| Crystal color, morphology | yellow, needle |
| Crystal size | 0.090 × 0.090 × 0.060 mm³ |
| Theta range for data collection | 2.193 to 26.373° |
| Index ranges | −14 £ h £ 14, −17 £ k £ 19, −19 £ l £ 25 |
| Reflections collected | 40713 |
| Independent reflections | 7504 [R(int) = 0.0532] |
| Observed reflections | 5941 |
| Completeness to theta = 25.242° | 99.9% |
| Absorption correction | Multi-scan |
| Max. and min. transmission | 0.7454 and 0.6663 |
| Refinement method | Full-matrix least-squares on F² |
| Data / restraints / parameters | 7504 / 0 / 412 |
| Goodness-of-fit on F² | 1.053 |
| Final R indices [I > 2sigma(I)] | R1 = 0.0409, wR2 = 0.1060 |
| R indices (all data) | R1 = 0.0561, wR2 = 0.1154 |
| Extinction coefficient | n/a |
| Largest diff. peak and hole | 0.635 and −0.288 eÅ³ |

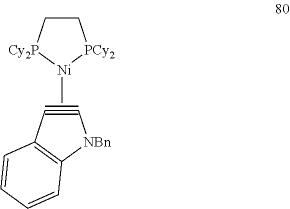

The general procedure was followed to give complex 80 in 90% yield. Single crystals suitable for X-ray diffraction analysis were obtained from a THF solution layered with pentane. ¹H NMR (THF-d₈) δ 7.43-7.38 (m, 1H), 7.22-7.15 (m, 4H), 7.10 (t, J_{HH}=7.0 Hz, 1H), 6.96-6.89 (m, 1H), 6.80-6.74 (m, 2H), 5.35 (s, 2H), 2.19 (d, J_{HH}=13.1 Hz, 2H), 2.06-1.04 (overlapping peaks, 46H). ³¹P{¹H} NMR (THF-d₈) δ 90.19 (d, J=4.7 Hz), 85.65 (d, J=4.7 Hz). ¹³C{¹H} NMR (THF-d₈) δ 168.05 (dd, J_{PC}=91.7, 10.5 Hz), 157.10, 142.10, 128.92, 127.99, 127.21, 127.15 (overlapping multiplet), 126.18 (dd, J_{PC}=83.3, 16.9 Hz), 123.37, 119.78, 117.96, 110.56, 53.08, 36.10 (dd, J=56.9, 21.0 Hz), 30.83 (d, J_{PC}=4.4 Hz), 30.58 (d, J_{PC}=3.2 Hz), 30.26 (d, J_{PC}=2.6 Hz), 28.38-27.66 (overlapping peaks), 27.24 (d, J_{PC}=7.7 Hz), 23.15 (dd, J_{PC}=22.4, 18.2 Hz), 22.59 (dd, J_{PC}=23.5, 16.9 Hz). HRMS (ESI⁺) 686.3549 calculated for C₄₁H₅₉NNiP₂ [M+H]⁺, found 686.3527.

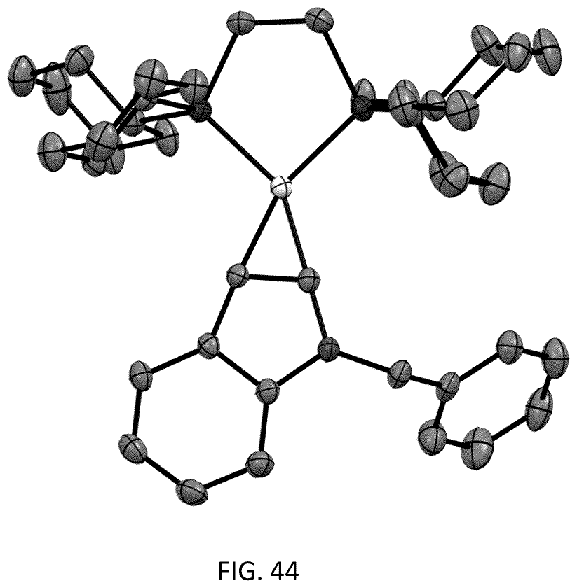

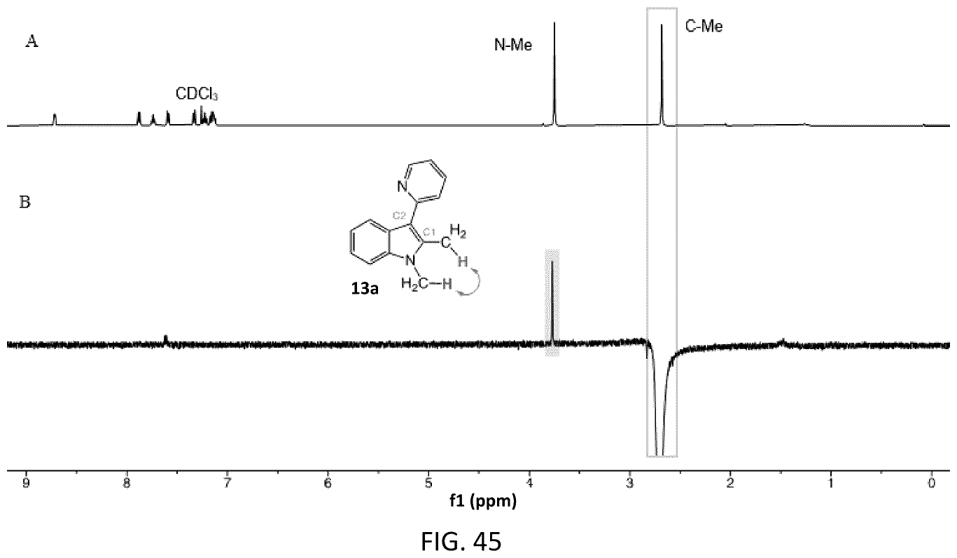

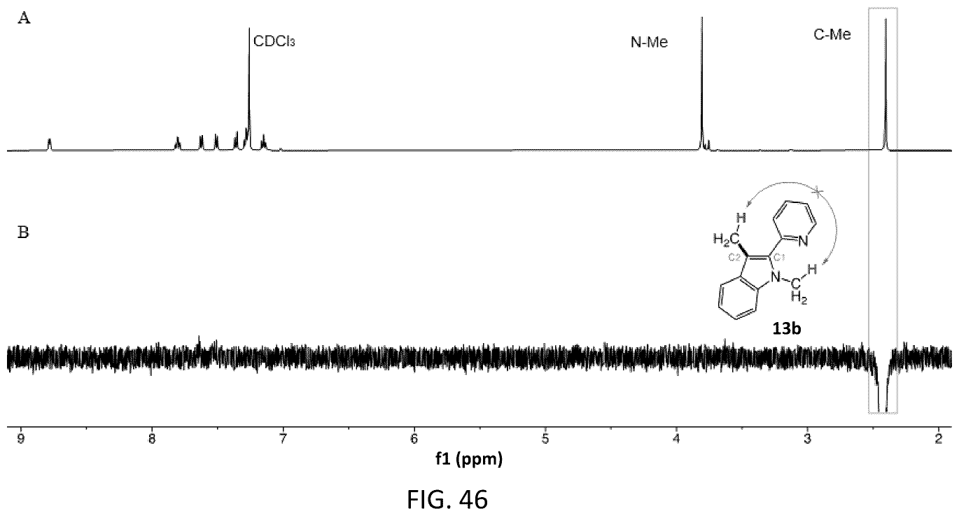

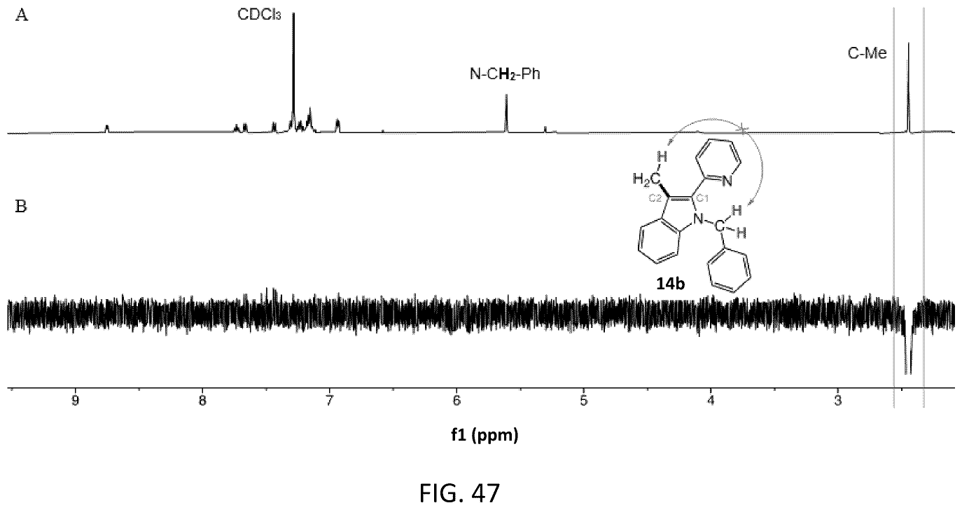

The crystal data for complex 80 is summarized below. The ORTEP drawing is depicted in FIG. 44.

TABLE 10
Crystal data and structural refinement for complex 80.

| Empirical formula | C₄₁ H₅₉ N Ni P₂ | |
|---|---|---|
| Formula weight | 686.54 | |
| Temperature | 122(2) K | |
| Wavelength | 0.71073 Å | |
| Crystal system | Monoclinic | |
| Space group | P2¹/c | |
| Unit cell dimensions | a = 11.8025(6) A | a = 90° |
| | b = 15.8222(10) Å | b = 102.485(2) ° |
| | c = 20.2336(10) Å | g = 90° |

| | |
|---|---|
| Volume | 3689.1(4) Å³ |
| Z | 4 |
| Density (calculated) | 1.236 Mg/m³ |
| Absorption coefficient | 0.642 mm⁻¹ |
| F(000) | 1480 |
| Crystal color, morphology | yellow, needle |
| Crystal size | 0.160 × 0.120 × 0.060 mm³ |
| Theta range for data collection | 2.186 to 30.516° |
| Index ranges | -16£ h £ 15, -22 £ k £ 22, -21 £ 1 £ 28 |
| Reflections collected | 42568 |
| Independent reflections | 11264 [R(int) = 0.0436] |
| Observed reflections | 8925 |
| Completeness to theta = 25.242∞ | 99.9% |
| Absorption correction | Semi-empirical from equivalents |
| Refinement method | Full-matrix least-squares on F² |
| Data / restraints / parameters | 11264 / 0 / 406 |
| Goodness-of-fit on F2 | 1.018 |
| Final R indices [I > 2sigma(I)] | R1 = 0.0432, wR2 = 0.1024 |
| R indices (all data) | R1 = 0.0598, wR2 = 0.1124 |
| Extinction coefficient | n/a |
| Largest diff. peak and hole | 0.717 and -0.359 eÅ³ |

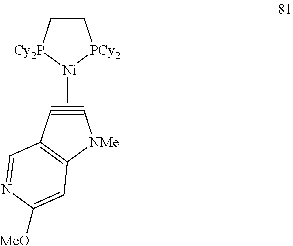

The general procedure was followed to give complex 81 in 89% yield. HRMS (ESI⁺) 641.3294 calculated for C₃₅H₅₆N₂NiOP₂ [M+H]⁺, found 641.3284.

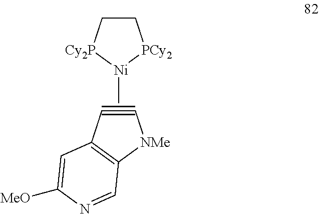

The general procedure was followed with minor modification. The brown semisolid which resulted from concentration of the filtered reaction mixture was triturated with hexamethyldisiloxane (HMDSO) to give complex 82 in 91% yield. ¹H NMR (THF-d₈) 7.77 (s, 1H), 6.60 (s, 1H), 3.82 (s, 3H), 3.79 (s, 3H), 2.21-0.61 (overlapping peaks, 48H). ¹³C{¹H} NMR (126 MHz, THF-d₈) δ 158.48, 153.36, 126.28, 100.77, 53.25, 36.07 (dd, J=44.4, 21.7 Hz), 35.59, 30.92, 30.52, 30.28 (d, J=20.8 Hz), 29.19, 28.22 (d, J=9.6 Hz), 27.86, 27.25. Note: Due to the stability of the complex in solution, attempts at longer ¹³C{¹H} NMR experiments did not improve the quality of spectra as the complex began to decompose over the time course of the experiment. Therefore, both aryne carbons and the two bridgehead carbons were not observable. ¹¹P{¹H} NMR (THF-d₈) δ 89.73 (d, J=3.7 Hz), 84.68 (d, J=3.7 Hz). HRMS (ESI⁺) 679.2952 calculated for C₃₅H₅₆N₂NiOP₂ [M+K]⁺, found 679.2950.

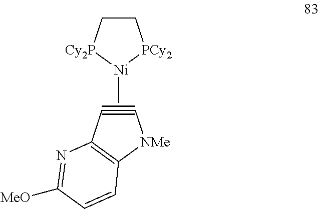

The general procedure was followed to give complex 83. Due to instability in solution, the complex was unable to be isolated. NMR spectra of the crude reaction are included. ³¹P{¹H} NMR (162 MHz, C₆D₆) δ 89.93 (d, J=4.0 Hz), 82.88 (d, J=4.7 Hz).

### Characterization of Ni-Bound Borate Complex 84

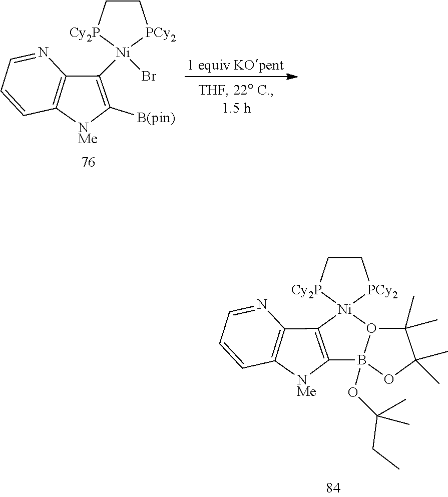

Single crystals suitable for X-ray diffraction analysis were obtained. ¹H NMR (500 MHz, C₆D₆) δ 8.50 (d, J=4.6 Hz, 1H), 7.21 (d, J=7.9 Hz, 1H), 6.84 (dd, J=7.9, 4.6 Hz, 1H), 4.30 (q, J=12.5 Hz, 1H), 4.06 (s, 3H), 3.63-3.56 (m, 2H), 2.97 (t, J=10.1 Hz, 1H), 2.64 (t, J=9.8 Hz, 1H), 2.46 (q, J=12.0 Hz, 2H), 2.27-1.49 (m, overlapping with B(pin) methyl signal, 29H), 1.45-0.85 (m, overlapping with O'Pent methyl signal, 52H), 0.62 (d, J=12.7 Hz, 1H). ¹³C{¹H} NMR (126 MHz, C₆D₆) δ 152.75, 139.15, 132.77 (d, J_{CP}=5.0 Hz), 116.10 (dd, J_{CP}=80.9, 54.6 Hz), 114.05, 111.99, 71.52, 40.30 (d, J_{CP}=3.8 Hz), 40.03 (d, J_{CP}=3.6 Hz), 38.52, 37.00-36.74 (m, overlapping multiplets), 36.70, 36.47 (d, J_{CP}=6.0 Hz), 33.67 (d, J_{CP}=11.4 Hz), 33.14 (d, J_{CP}=7.3 Hz), 32.19, 31.11 (d, J_{CP}=6.8 Hz), 30.22, 29.74 (d, J_{CP}=5.6 Hz), 29.14 (d, J_{CP}=16.5 Hz), 28.83, 28.44 (dd, J_{CP}=13.5, 7.4 Hz), 28.22-27.65 (m, overlapping multiplets), 27.41-27.05 (m, overlapping multiplets), 26.64 (d, J_{CP}=8.1 Hz), 26.00 (d, J_{CP}=4.3 Hz), 22.73, 20.47 (t, J_{CP}=20.4 Hz), 18.61 (dd, J_{CP}=23.2, 8.8 Hz), 14.28, 10.54. ³¹P{¹H} NMR (203 MHz, C₆D₆) δ 72.91 (d, J_{PP}=12.7 Hz), 45.23 (d, J_{PP}=13.0 Hz). ¹¹B{¹H} NMR (161 MHz, C₆D₆) δ 8.18. HRMS (ESI⁺) 737.4046 calculated for C₄₅H₇₇B₂N₂NiO₃P₂[M-O'pent]⁺, found 737.4046.

### Example 4: Synthetic Utility of the Heteroaryne Building Blocks

### A. Scaffold Swap

### Scheme 1. Scaffold Swap of Ambien Using Heteroaryne Precursors

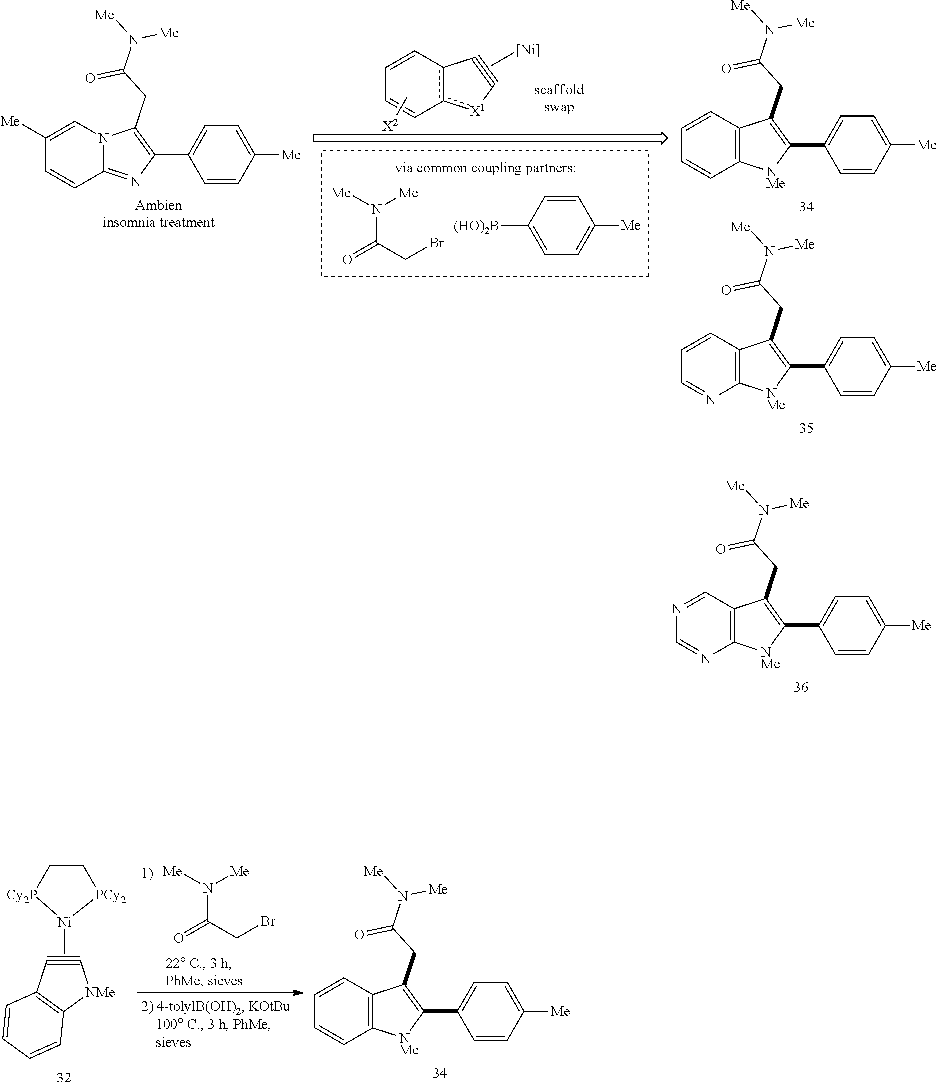

General Procedure for Scaffold Swapping of Ambien Derivatives. In a glovebox, an oven dried vial was charged with complex 32 (46 mg, 0.075 mmol, 1.0 equiv), 4 Å molecular sieves, and toluene (3 mL, 0.2 M). 2-bromo-dimethylacetamide (12.1 μL, 0.11 mmol, 1.5 equiv) and the reaction was stirred at 22° C. for 3 hours. A separate vial was charged with KotBu (26 mg, 0.23 mmol, 3 equiv), 4-methylphenylboronic acid (31 mg, 0.23 mmol, 3 equiv) and toluene (1 mL). The mixture was stirred at 22° C. for 5 minutes then added to the aryne reaction vial. The mixture was stirred at 100° C. After 18 hours, the vial was allowed to cool to room temperature and taken out of the glovebox. The mixture was diluted with EtOAc (20 mL) and washed with aqueous NH₄Cl solution (20 mL). The organic layer was dried (MgSO₄), filtered, and evaporated to dryness.

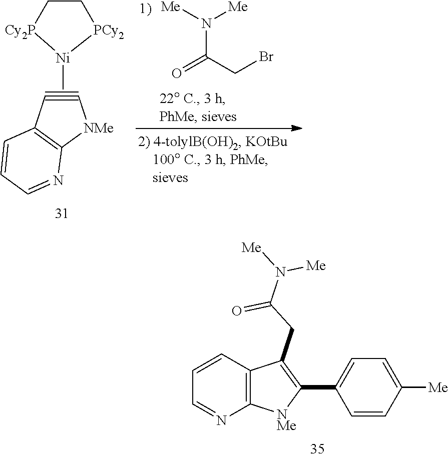

The general procedure was followed. The product was detected by GCMS.

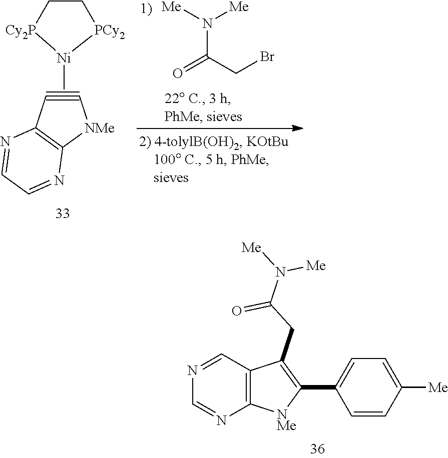

Procedure followed from above.

### B. Diversification of Heteroaryne Precursor Using Different Coupling Partners

### Scheme 2. Reactivity of Heteroaryne

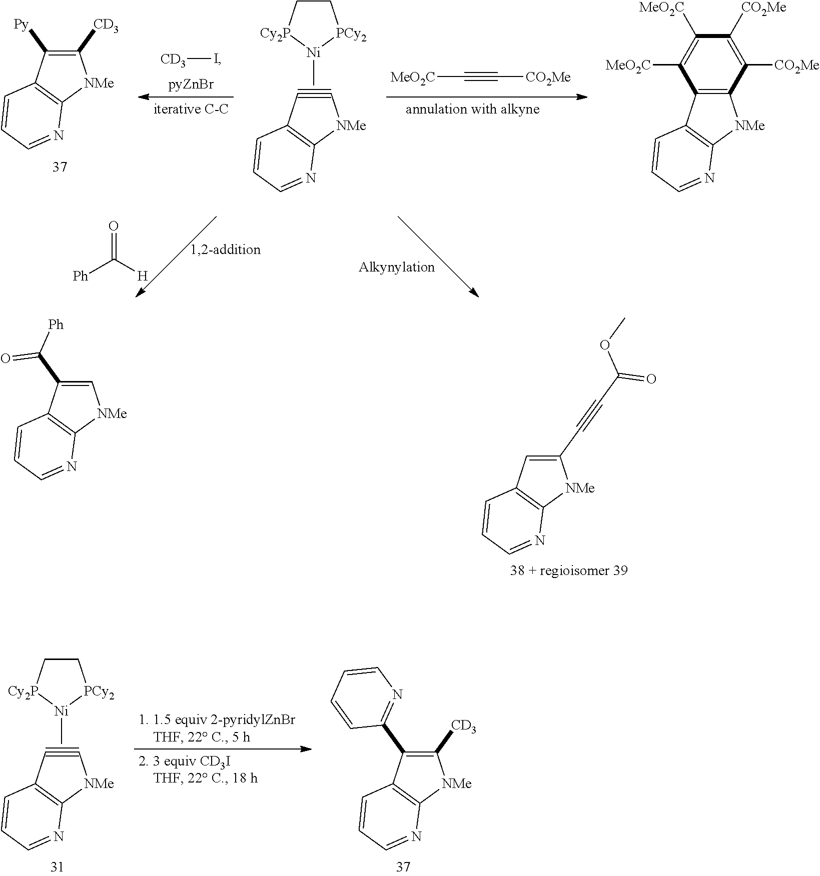

In a glovebox, an oven dried vial was charged with complex 31 (50 mg, 0.082 mmol, 1.0 equiv), 4 Å molecular sieves, and THF (3 mL, 0.3 M). 2-pyridylzinc bromide (0.5 M in THF, 245 νL, 0.12 mmol, 1.5 equiv) and the reaction was stirred at 22° C. After 5 hours, d₃-iodomethane (15.3 μL, 0.24 mmol, 3.0 equiv) was added and the reaction was stirred at 22° C. After 18 hours, the vial was taken out of the glovebox. The mixture was diluted with EtOAc (20 mL) and washed with aqueous NH₄Cl solution (20 mL). The organic layer was dried (MgSO₄), filtered, and evaporated to dryness. Purification by preparative TLC (10% EtOAc/5% triethylamine/85% hexanes) afforded compound 37 in 52% yield. ¹H NMR (500 MHz, CDCl₃): δ 8.72 (ddd, J=5.0, 1.9, 1.0 Hz, 1H), 8.30 (dd, J=4.7, 1.6 Hz, 1H), 8.17 (dd, J=7.8, 1.6 Hz, 1H), 7.75 (td, J=7.7, 1.9 Hz, 1H), 7.51 (dt, J=7.9, 1.1 Hz, 1H), 7.16 (ddd, J=7.5, 5.0, 1.2 Hz, 1H), 7.10 (dd, J=7.9, 4.8 Hz, 1H), 3.87 (s, 3H). ²H NMR (61 MHz, CH₂Cl₂): δ 2.69 (s, 3H). ¹³C{¹H}NMR (126 MHz, CDCl₃): δ 154.66, 149.64, 147.95, 142.12, 136.82, 136.38, 127.30, 123.29, 120.31, 119.49, 116.45, 111.38, 28.19, 8.17.

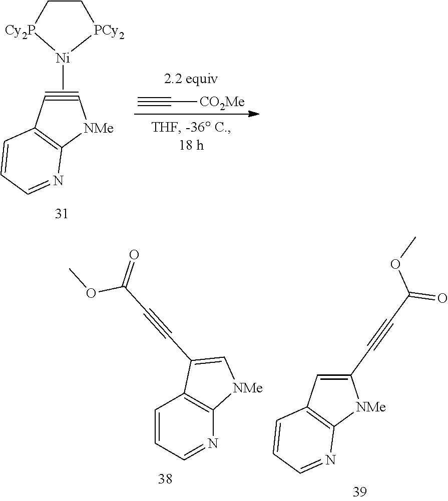

In a glovebox, an oven dried vial was charged with complex 31 (55 mg, 0.090 mmol, 1.0 equiv), 4 Å molecular sieves, and cold THF (−36° C., 4 mL, 0.2 M). Methyl propiolate (17 μL, 0.20 mmol, 2.2 equiv) and the reaction was stirred at −36° C. After 18 hours, the vial was taken out of the glovebox. The mixture was diluted with EtOAc (20 mL) and washed with aqueous NH₄Cl solution (20 mL). The organic layer was dried (MgSO₄), filtered, and evaporated to dryness. Purification by flash chromatography (10% EtOAc/hexanes) afforded compound 38 in 35% yield and compound 39 in 15% yield.

Compound 38: ¹H NMR (500 MHz, CDCl₃): δ 8.41 (dd, J=4.7, 1.6 Hz, 1H), 8.08 (dd, J=7.8, 1.6 Hz, 1H), 7.64 (s, 1H), 7.20 (dd, J=7.9, 4.7 Hz, 1H), 3.92 (s, 3H), 3.85 (s, 3H). ¹³C NMR (126 MHz, CDCl₃): δ 154.84, 147.13, 144.58, 136.30, 128.40, 121.36, 117.34, 92.00, 83.71, 81.93, 52.48, 31.72.

Compound 39: ¹H NMR (500 MHz, CDCl₃): δ 8.45 (dd, J=4.6, 1.6 Hz, 1H), 7.92 (dd, J=8.0, 1.5 Hz, 1H), 7.11 (dd, J=7.9, 4.6 Hz, 1H), 7.00 (s, 1H), 3.95 (s, 3H), 3.88 (s, 3H). ¹³C NMR (126 MHz, CDCl₃): δ 154.20, 148.23, 146.48, 130.02, 119.37, 118.95, 117.09, 110.59, 87.61, 78.24, 53.10, 29.65.

### Ambiphilic Methylation of 31

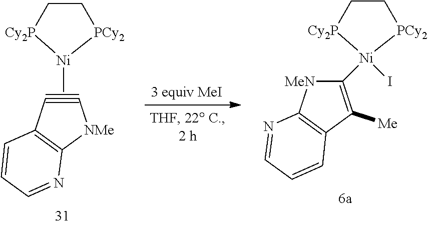

In a glovebox, an oven dried vial was charged with complex 31 (61.1 mg, 0.1 mmol, 1.0 equiv), and THE (0.5 mL, 0.2 M). Iodomethane (18.7 μL, 0.300 mmol, 3 equiv) was added and the reaction was stirred at 22° C. After 2 hours, the mixture was concentrated under vacuum. The residue was washed with pentane and dried under vacuum to give compound 6a as a red solid in >98% yield (74.5 mg). The other regioisomer of the product was not observed. Single crystals suitable for X-ray crystallography were grown from a THF solution of the complex with slow vapor diffusion of pentane at 22° C.

¹H NMR (500 MHz, CDCl₃): δ 7.87 (d, J=4.9 Hz, 1H), 7.38 (d, J=7.5 Hz, 1H), 6.79 (dd, J=7.6, 4.8 Hz, 1H), 4.14 (s, 3H), 2.48 (s, 3H), 2.54-2.16 (overlapping peaks, 6H), 2.10-1.00 (overlapping peaks, 42H). ¹³C{¹H} NMR (126 MHz, CDCl₃): δ 153.36 (d, J_{PC}=5.2 Hz), 145.74 (dd, J_{PC}=88.2, 33.3 Hz), 136.34, 124.38 (d, J_{PC}=4.5 Hz), 120.34, 113.67 (d, J_{PC}=3.7 Hz), 112.73, 35.96, 35.78 (N-Me), 35.64, 35.63, 34.79 (d, J_{PC}=23.8 Hz), 30.11, 30.02, 29.93, 29.53, 29.22 (d, J_{PC}=3.5 Hz), 28.92, 27.91, 27.63 (d, J_{PC}=4.3 Hz), 27.55-27.00 (overlapping peaks), 26.28 (d, J_{PC}=4.0 Hz), 26.07, 22.94 (t, J_{PC}=20.8 Hz), 20.43 (dd, J_{PC}=20.6, 12.2 Hz), 13.69 (C-Me). ³¹P{¹H} NMR (CDCl₃): 71.09 (d, Jₚₚ=25.2 Hz), 65.72 (d, Jₚₚ=25.2 Hz). HRMS (ESI⁺): 625.3350 calculated for C₃₅H₅₇IN₂NiP₂ [M−I]⁺, found 625.3372.

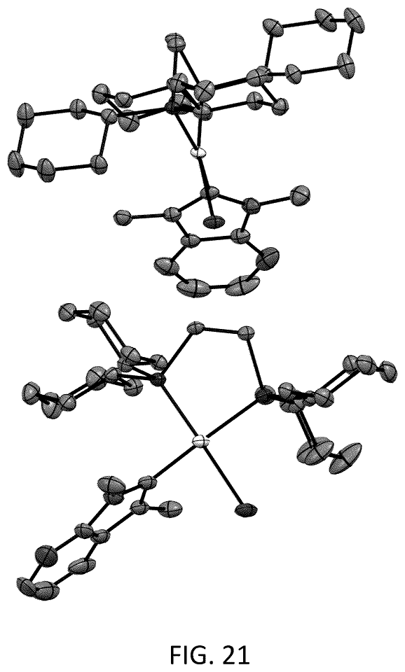

The crystal data for complex 6a is summarized below. The ORTEP drawing is depicted in FIG. 21.

PLATON/SQUEEZE was used to determine the solvent void volume and electron density within. These results corroborate nearly fully-occupied solvent positions. The solvent-corrected data by PLATON/SQUEEZE improved the R1>2%.

TABLE 11
Crystal data and structural refinement for complex 6a.

| Empirical formula | C₃₅ H₅₇ I N₂ Ni P₂ | |
|---|---|---|
| Formula weight | 753.37 | |
| Temperature | 100(2) K | |
| Wavelength | 0.71073 Å³ | |
| Crystal system | Triclinic | |
| Space group | P −1 | |
| Unit cell dimensions | a = 12.2463(6)Å | α = 81.691(2) ° |
| | b = 16.5668(7)Å | β = 74.580(2) ° |
| | c = 22.5085(9)Å | γ = 72.230(2) ° |

| | |
|---|---|
| Volume | 4182.2(3)Å3 |
| Z | 4 |
| Density (calculated) | 1.197 Mg/m³ |
| Absorption coefficient | 1.301 mm⁻¹ |
| F(000) | 1568 |
| Crystal color, morphology | yellow, needle |
| Crystal size | 0.150 × 0.100 × 0.080 mm³ |
| Theta range for data collection | 1.898 to 25.682° |
| Index ranges | −14 £ h £ 14, −20 £ k £ 20, −25 £ l £ 27 |
| Reflections collected | 74562 |
| Independent reflections | 15874 [R(int) = 0.0405] |
| Observed reflections | 13616 |
| Completeness to theta = 25.242° | 99.9% |
| Absorption correction | Semi-empirical from equivalents |
| Max. and min. transmission | 0.7461 and 0.6892 |
| Refinement method | Full-matrix least-squares on F² |
| Data / restraints / parameters | 15874 / 306 / 745 |
| Goodness-of-fit on F² | 1.024 |
| Final R indices [I > 2sigma(I)] | R1 = 0.0329, wR2 = 0.0736 |
| R indices (all data) | R1 = 0.0415, wR2 = 0.0788 |
| Extinction coefficient | n/a |
| Largest diff. peak and hole | 3.452 and −1.774 eÅ³ |

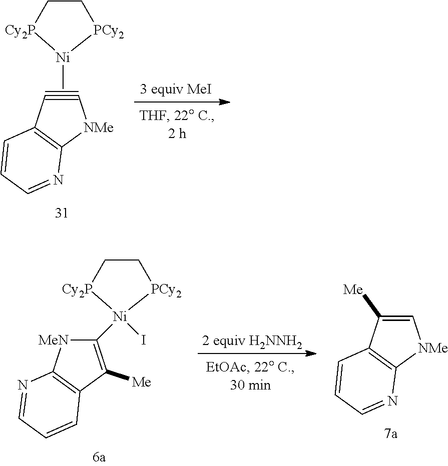

In a glovebox, an oven dried vial was charged with complex 31 (61.1 mg, 0.1 mmol, 1.0 equiv), and THE (0.5 mL, 0.2 M). Iodomethane (18.7 μL, 0.300 mmol, 3 equiv) was added and the reaction was stirred at 22° C. After 2 hours, the vial was taken out of the glovebox, and the reaction was diluted with EtOAc. Hydrazine (6.28 μL, 0.200 mmol, 2 equiv) was added and allowed to stir at 22° C. After 30 minutes, the organic mixture was concentrated under vacuum. The residue was dissolved in CDCl₃ and mesitylene (internal standard, 3.5 μL, 0.025 mmol) was added. The yield of the reaction was determined by NMR analysis to give compound 7a in 44% yield as the average of two duplicate reactions.

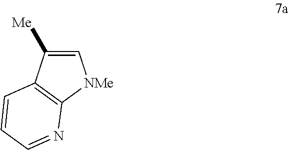

¹H NMR (500 MHz, CDCl₃): δ 8.32 (dd, J=4.7, 1.6 Hz, 1H), 7.84 (dd, J=7.8, 1.6 Hz, 1H), 7.03 (dd, J=7.8, 4.7 Hz, 1H), 6.94 (s, 1H), 3.83 (s, 3H, N—CH₃), 2.31 (s, 3H, Ar—CH₃). ¹³C{¹H} NMR (CDCl₃): 142.84, 127.01, 126.61, 121.10, 115.66, 114.80, 108.74, 31.00 (N-Me), 9.77 (Ar-Me). IR (cm⁻¹) 3041, 2916, 2861, 2340, 2244, 2195, 2167, 2151, 2039, 2018, 1996, 1980, 1877, 1602, 1576, 1543, 1491, 1381, 1338, 1241, 1205, 1152, 1123, 1067, 1034, 1014, 984, 936, 797, 606, 555, 490, 470, 440, 428. HRMS (ESI⁺) 147.0922 calculated for C₉H₁₀N₂ [M+H]+ found 147.0922.

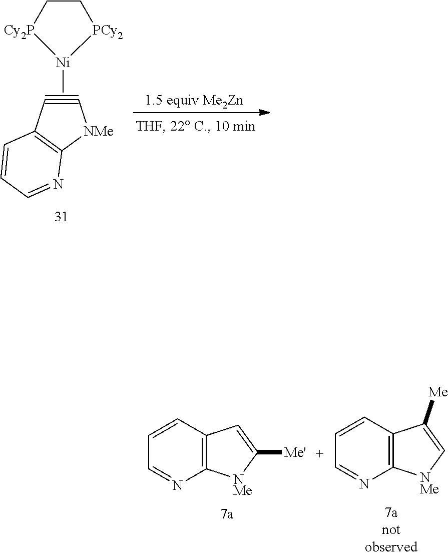

In a glovebox, an oven dried vial was charged with complex 31 (61.1 mg, 0.1 mmol, 1.0 equiv), 4 Å molecular sieves, and THF (0.5 mL, 0.2 M). Dimethyl zinc (1 M solution in heptane, 300 μL, 0.075 mmol, 3 equiv) was added and the reaction was stirred at 22° C. After 10 minutes, the vial was taken out of the glovebox. The mixture was diluted with EtOAc (20 mL) and washed with aqueous NH₄Cl solution (20 mL). The organic layer was dried (MgSO₄), filtered, and evaporated to dryness. The residue was dissolved in CDCl₃ and mesitylene (internal standard, 3.5 μL, 0.025 mmol) was added. The yield of the reaction was determined by NMR analysis to give compound 7a′ in 61% yield as the average of two duplicate reactions.

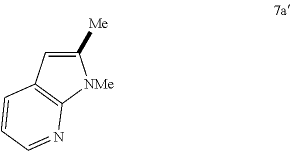

The material obtained from this method provided identical 1H NMR spectra to those previously reported. ¹H NMR (500 MHz, CDCl₃) δ 8.23 (dd, J=4.7, 1.5 Hz, 1H), 7.78 (dd, J=7.7, 1.5 Hz, 1H), 7.00 (dd, J=7.7, 4.8 Hz, 1H), 6.20 (s, 1H), 3.80 (s, 3H), 2.46 (d, J=1.0 Hz, 3H).

Reactions of Complex 31 with Electrophilic Coupling Partners

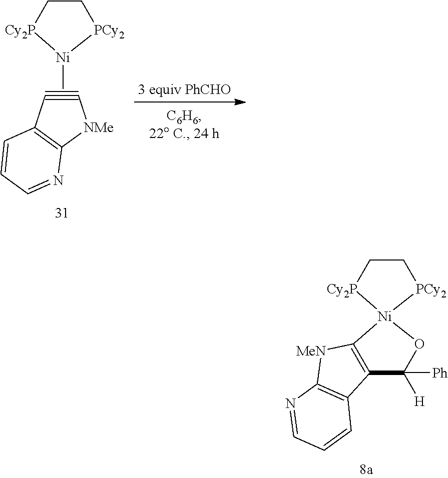

Complex 31 (122 mg, 0.2 mmol) was dissolved in benzene (0.5 mL, 0.4 M). Benzaldehyde (61 μL, 0.6 mmol, 3 equiv) was added and the reaction was stirred for 24 h at room temperature. The solvent was removed under vacuum affording a brown residue. Trituration with pentane (3×2 mL) precipitated out complex 8a as a yellow solid (96 mg, 68% yield).

¹H NMR (500 MHz, C₆D₆): δ 8.41 (d, J=4.6 Hz, 1H), 7.79 (d, J=7.5 Hz, 2H), 7.48 (d, J=7.6 Hz, 1H), 7.28 (t, J=7.5 Hz, 2H), 7.14 (t, J=7.3 Hz, 1H, overlapping with C₆D₅H residual solvent peak), 6.72 (dd, J=7.6, 4.7 Hz, 1H), 6.39 (s, 1H), 4.14 (s, 3H), 2.82 (d, J=12.2 Hz, 1H), 2.76 (d, J=10.5 Hz, 1H), 2.70 (d, J=12.6 Hz, 1H), 2.42 (d, J=12.6 Hz, 1H), 2.21-2.02 (m, 2H), 1.99-1.87 (m, 2H), 1.85-1.77 (m, 1H), 1.75-0.85 (overlapping peaks, 39H). ³¹P{¹H} NMR (126 MHz, C₆D₆): 72.09 (J_{AB}=43.9 Hz). ¹³C{¹H} NMR (126 MHz, C₆D₆): 163.96 (dd, J=114.1, 41.0 Hz), 152.40 (d, J=4.5 Hz), 151.70, 142.30, 139.32, 128.17, 127.94, 126.01, 124.03, 118.03, 114.20, 84.00 (d, J=7.2 Hz), 38.30 (dd, J=14.7, 5.6 Hz), 37.98 (dd, J=14.2, 5.1 Hz), 34.64 (dd, J=14.6, 5.5 Hz), 34.37 (dd, J=14.1, 5.3 Hz), 34.04, 33.24 (d, J=4.7 Hz), 32.66 (d, J=4.2 Hz), 30.36, 30.12 (dd, J=9.4, 4.7 Hz), 29.70, 29.29, 28.94, 27.93-27.17 (m), 26.66, 26.40, 23.60-22.53 (m), 18.67-16.62 (m). HRMS (ESI⁺): 717.3607 calculated for C₄₁H₆₁N₂OniP₂ [M+H]⁺, found 717.3581.

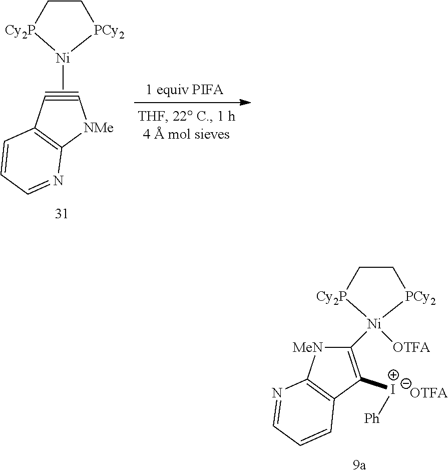

In a glovebox, an oven dried vial was charged with complex 31 (61.1 mg, 0.1 mmol, 1.0 equiv), 4 Å molecular sieves, and THF (3 mL, 0.03 M). [Bis(trifluoroacetoxy)iodo]benzene was added (45.0 mg, 0.1 mmol, 1.05 equiv) and the reaction was stirred at 22° C. for 1 hour. The mixture was concentrated under reduced pressure. The residue was washed with a mixture of diethyl ether and pentane and dried under vacuum, affording complex 9a as a yellow solid (69 mg, 66% yield). Single crystals suitable for X-ray crystallography were grown from a THF solution of the complex with slow vapor diffusion of Et₂O at 22° C.

¹H NMR (400 MHz, CDCl₃): δ 8.17 (d, J=4.8 Hz, 1H), 7.87 (d, J=8.1 Hz, 2H), 7.36 (m, 4H), 6.99 (dd, J=8.0, 4.7 Hz, 1H), 4.41 (s, 3H), 2.64-0.47 (m, 48H). ³¹P NMR (162 MHz, CDCl₃): δ 66.09 (d, J=40.5 Hz), 65.23 (d, J=40.5 Hz). ¹⁹F{¹H} NMR (376 MHz, CDCl₃): δ −74.45, −75.28. ¹³C{¹H} NMR (126 MHz, CDCl₃): δ 161.95 (q, J_{CF}=32.9 Hz, OTFA C═O), 161.07 (q, J_{CF}=36.6 Hz, OTFA C═O), 152.04, 152.01, 141.43, 131.77, 131.03, 130.77, 124.20, 123.32, 116.74, 116.45, 115.91, 81.01, 36.97, 36.77, 35.95, 35.78, 35.37, 35.04, 34.96, 34.87, 34.33, 34.25, 34.19, 32.67, 32.53, 32.17, 29.87, 29.30, 29.11, 28.96, 28.91, 28.78, 28.68, 28.51, 27.75, 27.66, 27.61, 27.19, 27.14, 27.00, 26.93, 26.90, 26.85, 26.77, 26.00, 25.84, 25.80, 24.65, 22.65, 22.47, 22.31, 20.58, 20.42, 16.43, 16.30, 15.41, 14.19. (Signals for C¹—Ni and CF₃ groups were not observed) HRMS (ESI⁺): 927.2397 calculated for C₄₂H₅₉F₃IN₂NiO₂P₂[M-TFA]⁺, found 927.2371.

NMR spectra contain an impurity identified as Ni(O₂CCF₃)₂(Cy₂PCH₂CH₂Pcy₂) by comparison with literature and ESI-MS. ¹H NMR (400 MHz, CDCl₃): δ 2.74-1.00 (m, 48H). ³¹P{¹H} NMR (162 MHz, CDCl₃): δ 78.09. HRMS (ESI⁺) 593.2430 calculated for C₂₈H₄₈F₃IN₂NiO₂P₂[M-TFA]⁺, found 593.2421.

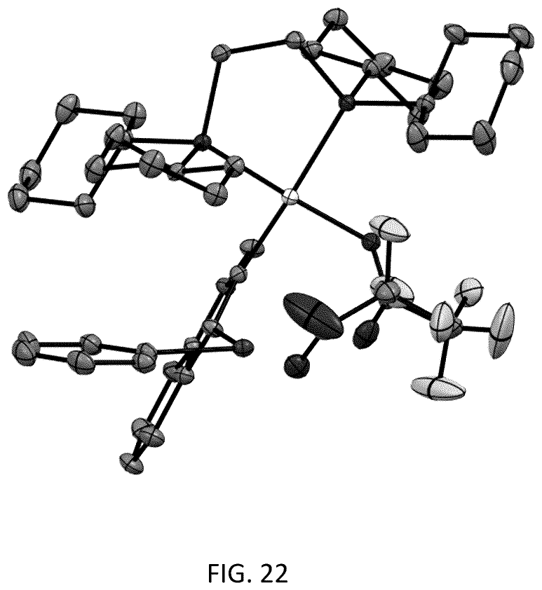

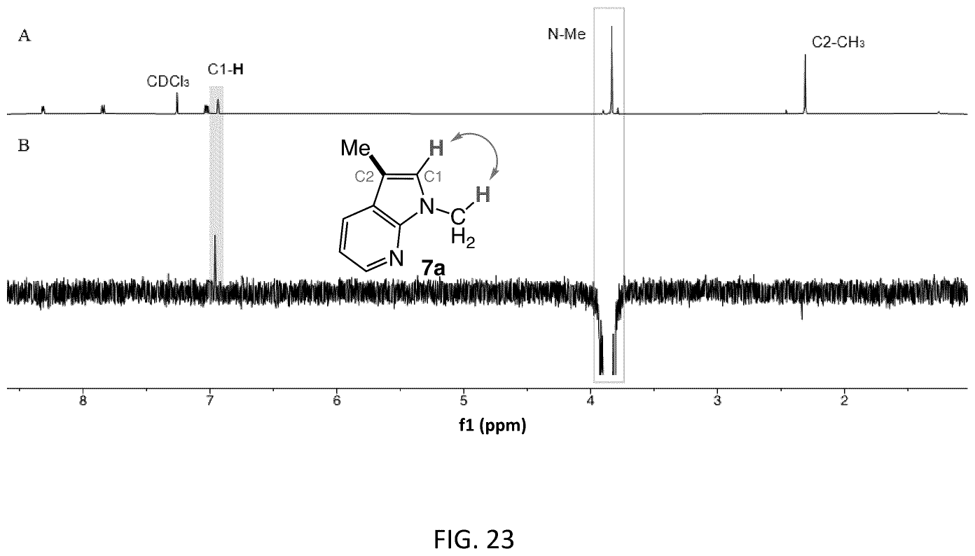

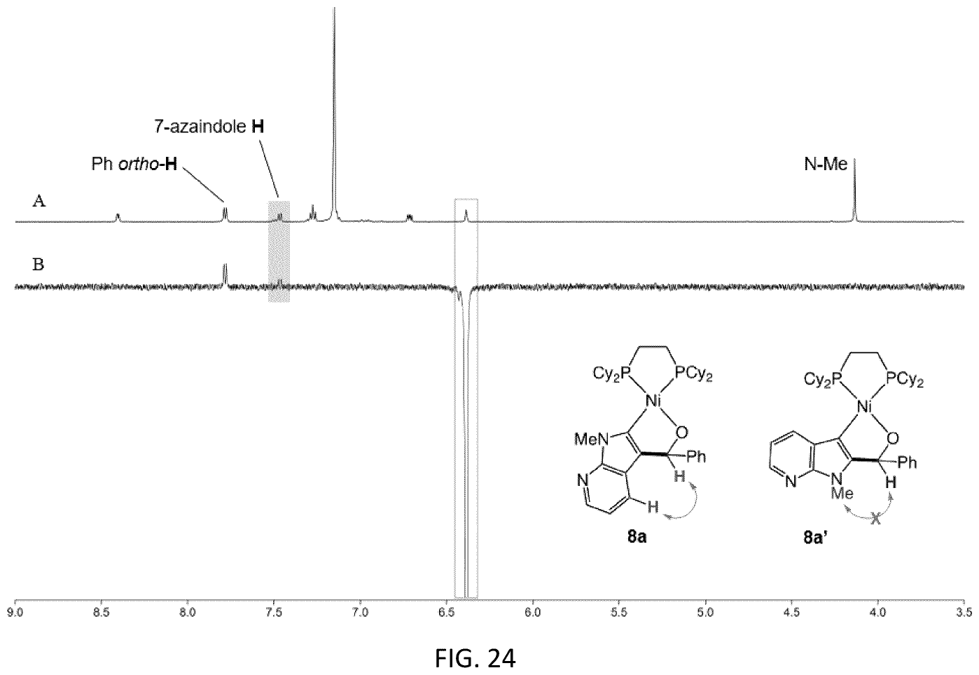

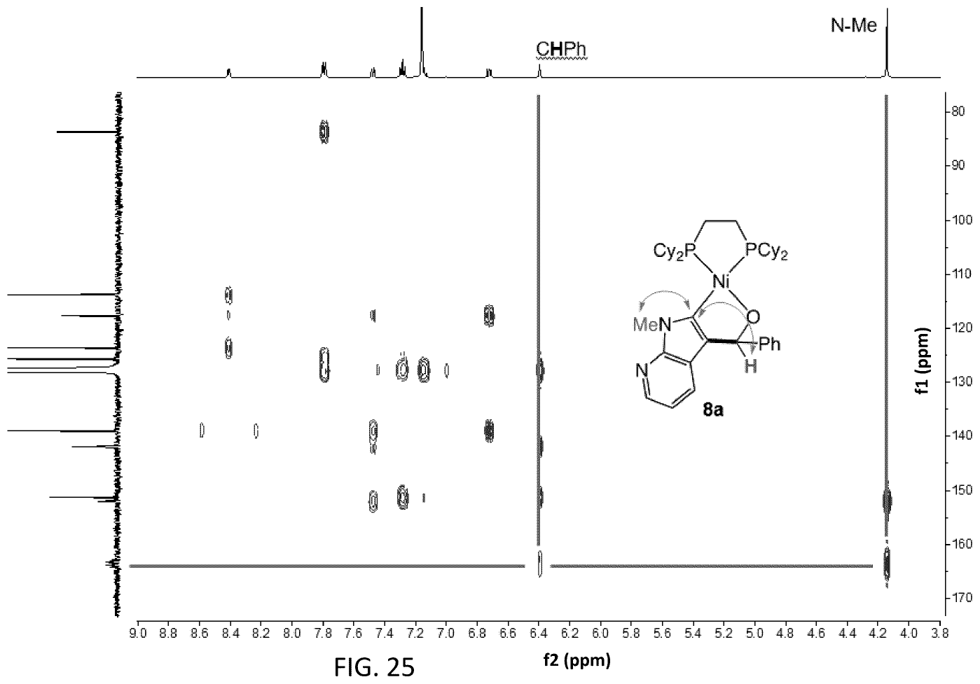

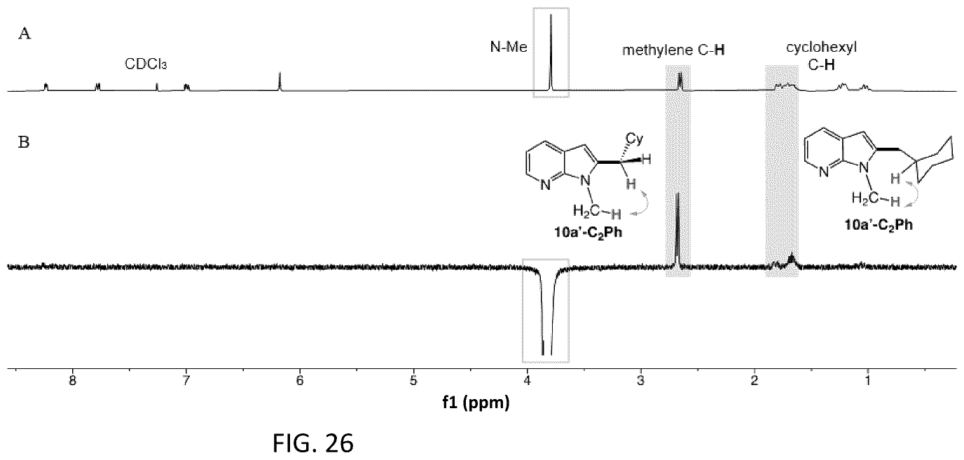

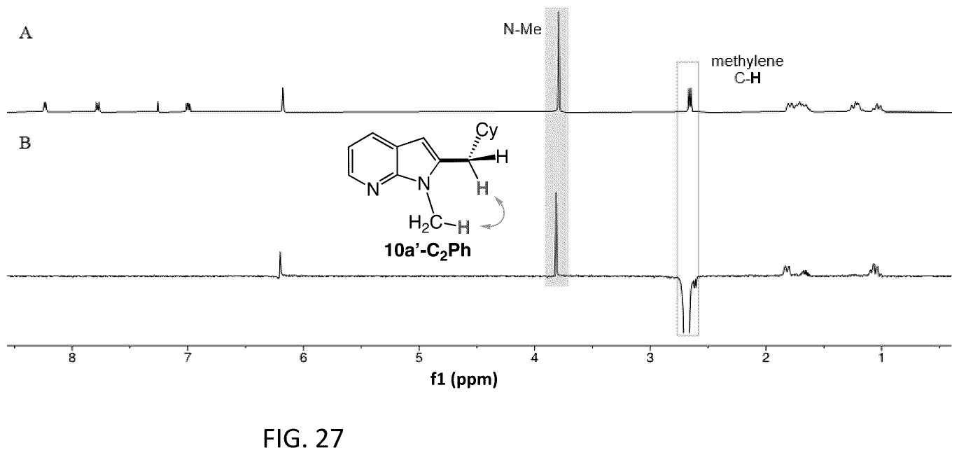

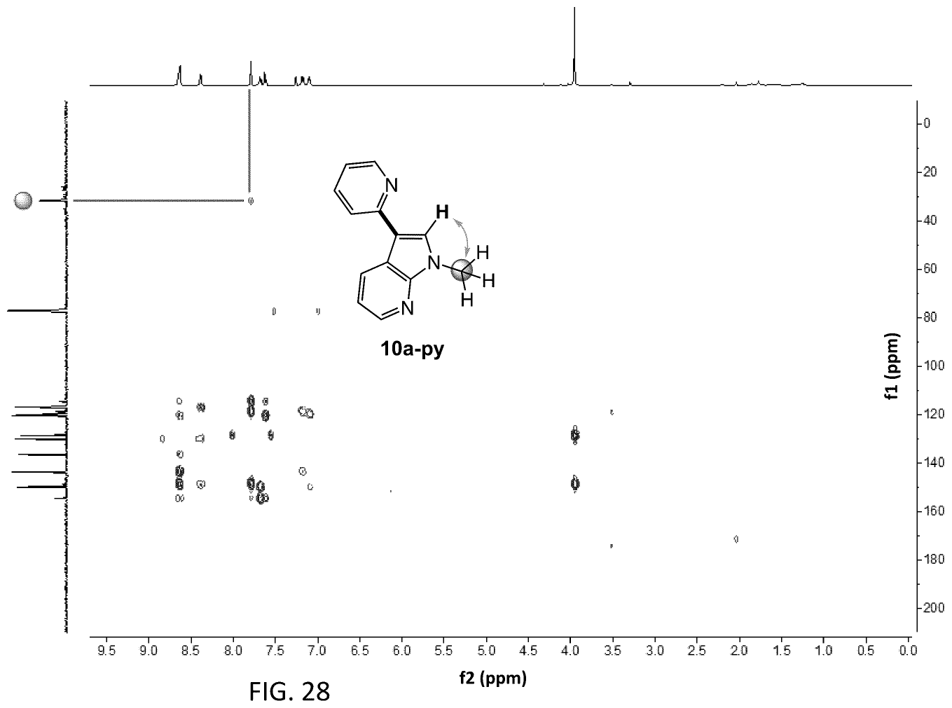

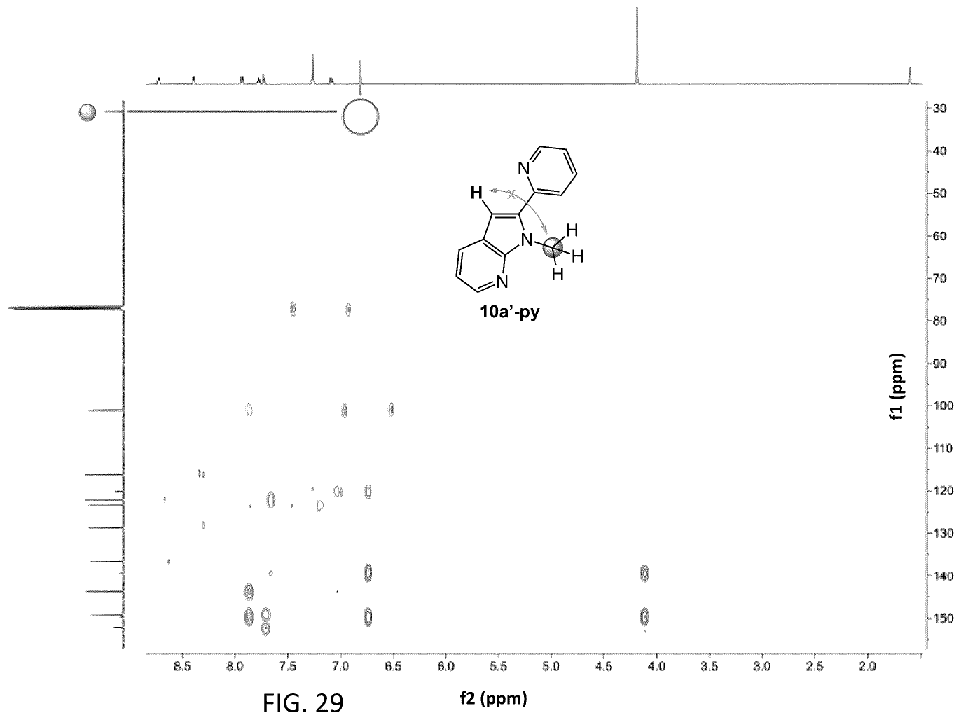

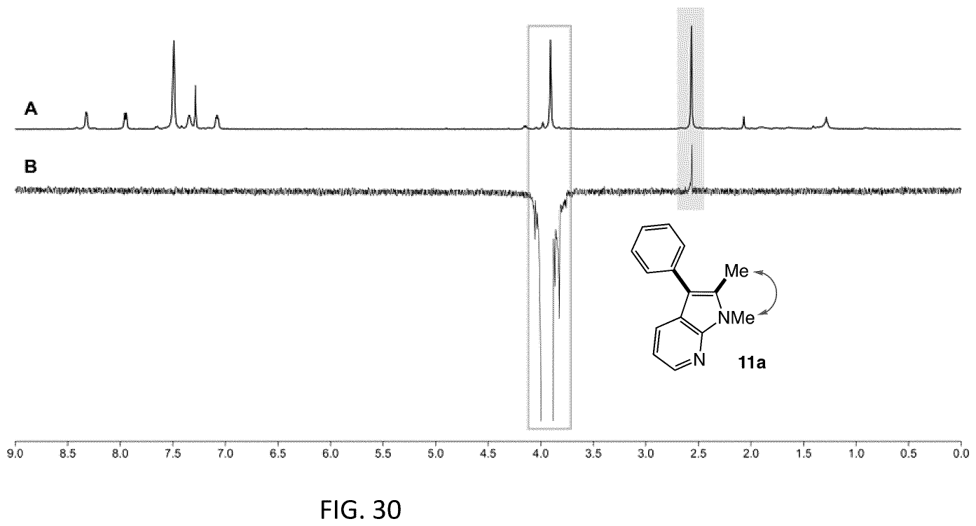

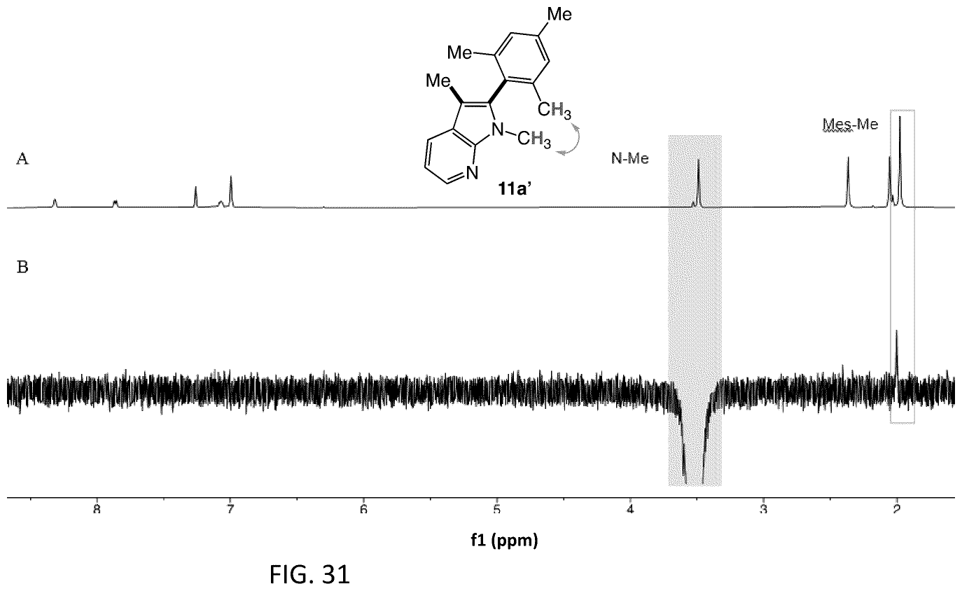

The crystal data for complex 9a is summarized below. The ORTEP drawing is depicted in FIG. 22.

The structure is the one suggested.

TABLE 12
Crystal data and structural refinement for complex 9a.

| Empirical formula | C₄₄ H₅₉ F₆ I N₂ Ni O₄ P₂ | |
|---|---|---|
| Formula weight | 1041.48 | |
| Temperature | 100(2) K | |
| Wavelength | 0.71073 Å³ | |
| Crystal system | Monoclinic | |
| Space group | P2₁/c | |
| Unit cell dimensions | a = 11.6566(6)Å | α = 90° |
| | b = 36.4553(18)Å | β = 100.853(2) ° |
| | c = 11.0101(6)Å | γ = 90° |

| | |
|---|---|
| Volume | 4595.0(4)Å³ |
| Z | 4 |
| Density (calculated) | 1.505 Mg/m³ |
| Absorption coefficient | 1.231 mm⁻¹ |
| F(000) | 2136 |
| Crystal color, morphology | yellow, block |
| Crystal size | 0.080 × 0.050 × 0.025 mm³ |
| Theta range for data collection | 1.865 to 33.257° |
| Index ranges | −17 £ h £ 17, −56 £ k £ 56, −16 € l £ 16 |
| Reflections collected | 90230 |
| Independent reflections | 17622 [R(int) = 0.0977] |
| Observed reflections | 12337 |
| Completeness to theta = 25.242° | 100.0% |
| Absorption correction | Semi-empirical from equivalents |
| Max. and min. transmission | 0.6869 and 0.7465 |
| Refinement method | Full-matrix least-squares on F² |
| Data / restraints / parameters | 17622 / 0 / 542 |
| Goodness-of-fit on F² | 1.027 |
| Final R indices [I > 2sigma(I)] | R1 = 0.0557, wR2 = 0.0933 |
| R indices (all data) | R1 = 0.0900, wR2 = 0.1056 |
| Extinction coefficient | n/a |
| Largest diff. peak and hole | 0.857 and −1.471 eÅ³ |

Reactions of Complex 31 with Nucleophilic Coupling Partners

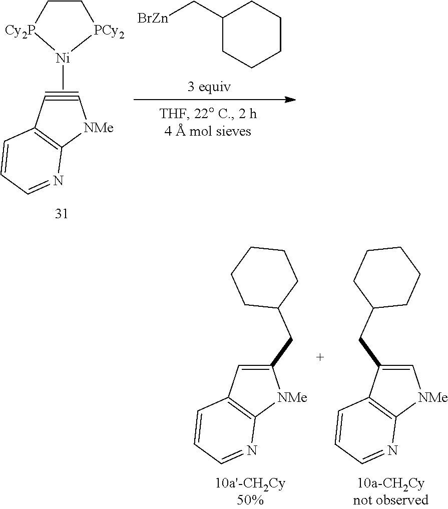

In a glovebox, an oven dried vial was charged with complex 31 (61.1 mg, 0.1 mmol, 1.0 equiv), 4 Å molecular sieves, and THF (5 mL, 0.02 M). (Cyclohexylmethyl)zinc bromide (0.5 M in THF, 600 μL, 0.30 mmol, 3 equiv) was added and the reaction was stirred at 22° C. After 2 hours, the vial was taken out of the glovebox. The mixture was diluted with EtOAc (20 mL) and washed with aqueous NH₄C1 solution (20 mL). The organic layer was dried (MgSO₄), filtered, and evaporated to dryness. Purification by column chromatography (5% EtOAc/hexanes gradient to 20% EtOAc/hexanes) afforded a mixture of compound 10a′-CH₂Cy and cyclohexyl methanol which was removed under reduced pressure (<200 mbar) for 3 days to give compound 10a′-CH₂Cy as a colorless oil in 50% yield (11.4 mg). Regioisomer 10a-CH₂Cy was not observed by crude NMR or during isolation.

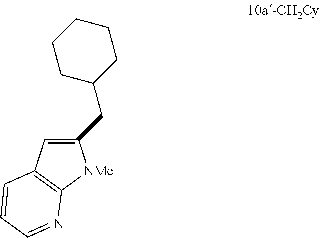

¹H NMR (500 MHz, CDCl₃): δ 8.24 (dd, J=4.7, 1.6 Hz, 1H), 7.78 (dd, J=7.7, 1.6 Hz, 1), 7.00 (dd, J=7.7, 4.7 Hz, 1), 6.18 (s, 1H), 3.79 (s, 3H), 2.66 (d, J=7.1 Hz, 2), 1.83-1.77 (m, 2H, overlapping peaks), 1.67 (m, 4H, overlapping peaks), 1.30-1.13 (m, 3H, overlapping peaks), 1.03 (m, 2H, overlapping peaks). ¹³C{¹H} NMR (126 MHz, CDCl₃): δ 148.68, 141.43, 140.88, 127.19, 120.72, 115.64, 98.06, 37.71, 35.04, 33.52, 28.28, 26.54, 26.35. IR (cm⁻¹) 3052, 3033, 2920, 2849, 1593, 1574, 1451, 1405, 1366, 1309, 1300, 1268, 1220, 1206, 918, 893, 798, 770, 577, 516. HRMS (ESI⁺): 229.1705 calculated for C₁₅H₂₀N₂ [M+H]⁺, found 229.1700.

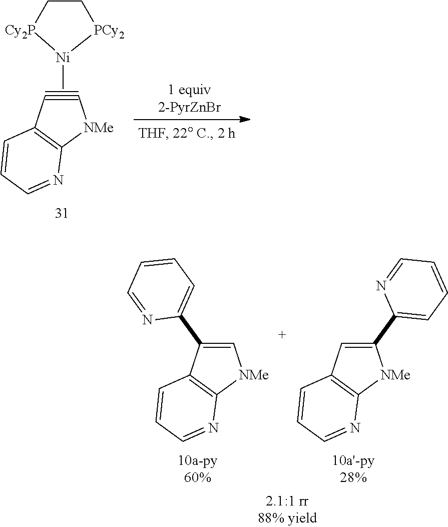

In a glovebox, an oven dried vial was charged with complex 31 (61.1 mg, 0.1 mmol, 1.0 equiv) and THF (5 mL, 0.02 M). 2-Pyridylzinc bromide (0.5 M in THF, 200 μL, 0.10 mmol, 1 equiv) was added and the reaction was stirred at 22° C. After 2 hours, the vial was taken out of the glovebox. The mixture was diluted with EtOAc (20 mL) and washed with aqueous NH₄Cl solution (20 mL). The organic layer was dried (MgSO₄), filtered, and evaporated to dryness. Purification by preparatory TLC (20% EtOAc/hexanes, 4 iterations) afforded compound 10a-py in 60% yield (12.5 mg) and compound 10a′-py in 28% yield (5.9 mg) for an 88% combined yield.

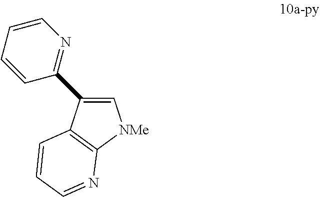

¹H NMR (500 MHz, CDCl₃): δ 8.70-8.58 (m, 2H), 8.39 (dd, J=4.7, 1.6 Hz, 1H), 7.79 (s, 1H), 7.68 (td, J=7.7, 1.8 Hz, 1H), 7.62 (d, J=8.0, 1.2 Hz, 1H), 7.18 (dd, J=7.9, 4.7 Hz, 1H), 7.10 (ddd, J=7.3, 4.9, 1.3 Hz, 1H), 3.95 (s, 3H). ¹³C{¹H} NMR (126 MHz, CDCl₃): δ 154.58, 149.78, 148.70, 143.56, 136.44, 129.95, 128.58, 120.46, 119.81, 118.70, 116.82, 114.48, 31.61. IR (cm⁻¹) 3049, 2946, 1730, 1565, 1533, 1428, 1403, 1372, 1322, 1303, 1278, 1231, 1151, 1132, 1109, 1092, 1028, 989, 907, 808, 789, 689, 662, 625, 593, 541, 498, 460, 431. HRMS (ESI⁺): 210.1031 calculated for C₁₃H₁₁N₃ [M+H]⁺, found 210.1034.

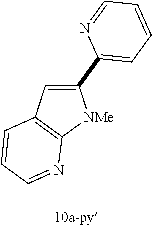

¹H NMR (500 MHz, CDCl₃): δ 8.72 (dt, J=4.8, 1.3 Hz, 1H), 8.39 (dd, J=4.7, 1.6 Hz, 1H), 7.93 (dd, J=7.8, 1.6 Hz, 1H), 7.78 (td, J=7.7, 1.8 Hz, 1H), 7.73 (dt, J=8.1, 1.2 Hz, 1H), 7.26 (ddd, J=7.3, 4.9, 1.4 Hz, overlapping with CHCl₃, 1H), 7.09 (dd, J=7.8, 4.6 Hz, 1H), 6.81 (s, 1H), 4.19 (s, 3H). ¹³C{¹H} NMR (126 MHz, CDCl₃): δ 152.24, 149.89, 149.43, 143.85, 139.56, 136.74, 128.88, 123.55, 122.37, 120.31, 116.35, 101.18, 30.85. IR (cm⁻¹) 3049, 3008, 2930, 2851, 1721, 1557, 1449, 1432, 1379, 1356, 1343, 1279, 1257, 1233, 1167, 1151, 1127, 1104, 1049, 990, 798, 740, 679, 626, 590, 545, 506, 455, 426, 413. HRMS (ESI⁺): 220.1031 calculated for C₁₃H₁₁N₃[M+H]⁺, found 210.1036.

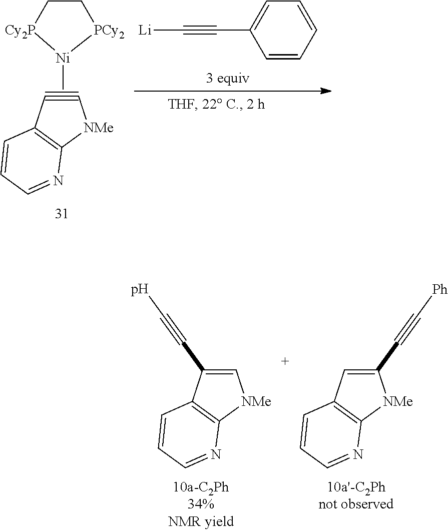

In a glovebox, an oven dried vial was charged with complex 31 (61.1 mg, 0.1 mmol, 1.0 equiv) and THE (3 mL, 0.03 M). Lithium phenylacetylide (1.0 M in THF, 150 μL, 0.15 mmol, 1.5 equiv) was added and the reaction was stirred at 22° C. After 2 hours, the vial was taken out of the glovebox. The mixture was diluted with DCM (3 mL) and washed with aqueous NH₄Cl solution (6 mL). The organic layer was dried (MgSO₄), filtered, and evaporated to dryness. The residue was dissolved in CDCl₃ and mesitylene (internal standard, 10 μL, 0.072 mmol) was added. The yield of the reaction was determined by NMR analysis. The reported 34% yield is the average of two duplicate reactions.

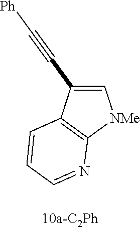

The material obtained from this method provided identical 1H NMR spectra to those previously reported. ¹H NMR (500 MHz, CDCl₃) δ 8.39 (dd, J=4.7, 1.6 Hz, 1H), 8.09 (dd, J=7.8, 1.6 Hz, 1H), 7.59-7.51 (m, 2H), 7.47 (s, 1H), 7.38-7.29 (m, 3H), 7.16 (dd, J=7.9, 4.7 Hz, 1H), 3.92 (s, 3H).

### Multicomponent Difunctionalizations of Complex 31

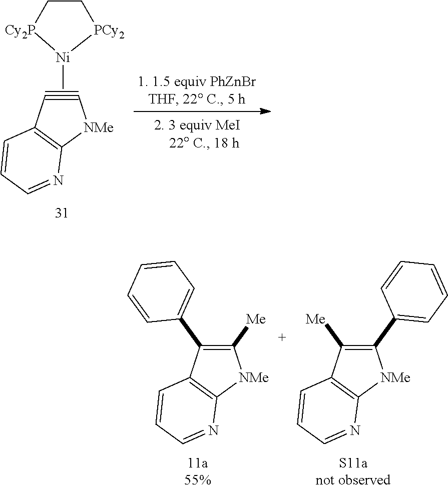

In a glovebox, an oven dried vial was charged with complex 31 (61 mg, 0.10 mmol, 1.0 equiv), and THF (0.5 mL, 0.2 M). 2-Phenyl zinc bromide (0.5 M in THF, 300 μL, 0.15 mmol, 1.5 equiv) was added and the reaction was stirred at 22° C. After 5 hours, methyl iodide (18.7 μL, 0.3 mmol, 3.0 equiv) was added and the reaction was stirred at 22° C. After 18 hours, the vial was taken out of the glovebox. The mixture was diluted with EtOAc (20 mL) and washed with aqueous NH₄Cl solution (20 mL). The organic layer was dried (MgSO₄), filtered, and evaporated to dryness. Purification by preparative TLC (10% EtOAc/90% hexanes, 3 iterations) afforded compound 11a as a white solid in 55% yield (12.2 mg).

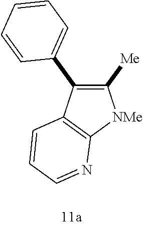

¹H NMR (CDC3, 500 MHz): 8.30 (dd, J=4.8, 1.6 Hz, 1H), 7.93 (dd, J=7.8, 1.6 Hz, 1H), 7.48-7.45 (m, 4H), 7.32 (tt, J=6.3, 3.0 Hz, 1H), 7.06 (dd, J=7.8, 4.7 Hz, 10), 3.88 (s, 3H), 2.55 (s, 3H). ¹³C{¹H} NMR (CDCl₃): 147.94, 141.97, 134.97, 134.21, 129.60, 128.80, 126.76, 126.20, 120.06, 116.01, 112.34, 28.41, 11.34. IR (cm⁻¹) 3053, 2931, 2853, 1568, 1550, 1497, 1488, 1436, 1402, 1379, 1358, 1343, 1252, 1179, 1155, 1130, 1099, 1076, 1033, 919, 900, 849, 795, 684, 667, 640, 623, 585, 569, 536, 493, 473, 460, 446, 429, 413. HRMS (ESI⁺): 223.1235 calculated for C₁₅H₁₄N₂₃ [M+H]⁺, found 223.1245.

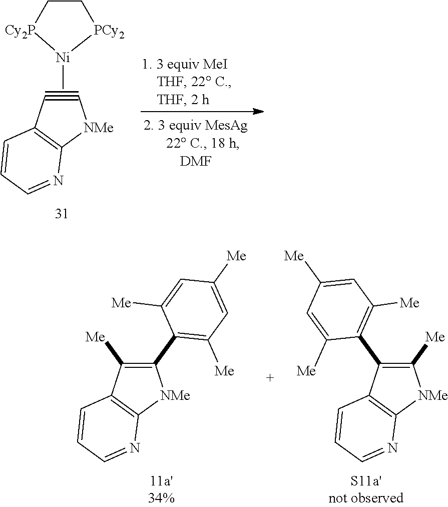

Complex 31 (183 mg, 0.300 mmol, 1 equiv) was dissolved in THF (3.0 mL, 0.1 M). MeI (56 μL, 0.900 mmol, 3 equiv) was added and the reaction was stirred for 2 h at room temperature. Once complex 31 was consumed and complex 6a was formed (determined by ³¹P{¹H} NMR spectroscopy), the solvent and excess MeI were removed under vacuum. The resulting red solid was washed with pentan (3×2 mL) to give complex 6a.

MesAg was prepared using following the literature procedure. A separate vial was charged with complex 6a (75.3 mg, 0.100 mmol, 1 equiv) and MesAg (68.1 mg, 0.300 mmol, 3 equiv) and DMF (5 mL, 0.02 M). The reaction was covered with foil to protect the reaction from light and allowed to stir at 22 C. After 18 h, the reaction was taken out of the glovebox, diluted with EtOAc (30 mL) and washed with 1M LiCl solution (2×30 mL). The residue was dissolved in CDCl₃ and CH₂Br (internal standard, 6.0 μL, 0.086 mmol) was added. The yield of the reaction was determined by NMR analysis. The reported yield is the average of two duplicate reactions. Purification by preparative TLC (20% EtOAc/hexanes, 2 iterations) afforded compound 11a′ as an off-white solid in 30% isolated yield (7.9 mg).

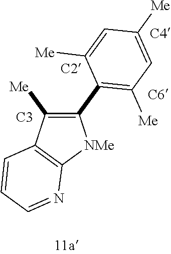

¹H NMR (500 MHz, CDCl₃) δ 8.32 (d, J=5.0 Hz, 1H), 7.86 (d, J=7.7 Hz, 1H), 7.07 (dd, J=7.9, 4.9 Hz, 1H), 6.99 (s, 2H), 3.49 (s, 3H, Nme), 2.37 (s, 3H, C3-Me), 2.06 (s, 3H, C4′-Me), 1.98 (s, 6H, C2′-Me and C6′-Me). ¹³C{¹H} NMR (126 MHz, CDCl₃) δ 148.36, 142.02, 138.66, 136.74, 128.24, 127.76, 126.37, 121.30, 115.01, 106.18, 99.12, 28.52, 21.38, 20.05, 8.82. IR (cm⁻¹) 2981, 2854, 1732, 1613, 1556, 1482, 1301, 1260, 1220, 1164, 1135, 1011, 907, 852, 789, 739, 632, 589, 564, 550, 484, 452. HRMS (ESI⁺): 265.1705 calculated for C₁₈H₂₁N₂ [M+H]⁺, found 265.1696.

Reactions of Complex 31 with Enophiles

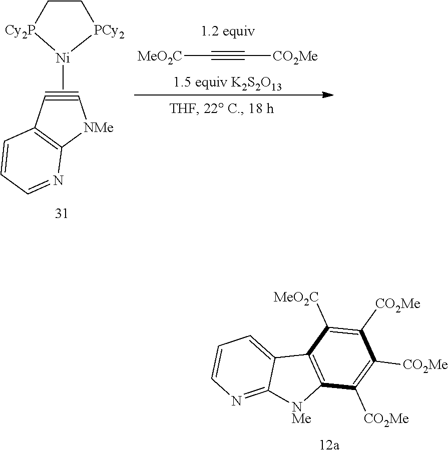

In a glovebox, an oven dried vial was charged with complex 31 (61.1 mg, 0.1 mmol, 1.0 equiv), potassium persulfate (40.5 mg, 0.15 mmol, 1.5 equiv), 4 Å molecular sieves, and THF (1.25 mL, 0.08 M). A separate vial was charged with dimethylacetylenedicarboxylate (15.5 μL, 0.12 mmol, 1.2 equiv) and THF (1.25 mL, 0.1 M). The DMAD solution was added to the aryne vial in 4 separate 250 μL aliquots every 15 minutes for 1 hour. The reaction was allowed to stir at 22° C. for an additional 18 hours. The solvent was evaporated under vacuum. The residue was dissolved in CDCl₃ and mesitylene (internal standard, 10 μL, 0.072 mmol) was added. The yield of the reaction was determined by NMR analysis. The reported 23% yield is the average of two duplicate reactions.

¹H NMR (400 MHz, CDCl₃): δ 8.63 (dd, J=4.7, 1.6 Hz, 1H), 8.40 (dd, J=8.0, 1.6 Hz, 1H), 7.31-7.27 (dd, overlapping with CDCl₃, J=2.43 Hz, 1H), 4.07 (s, 3H), 4.04 (s, 3H), 3.99 (s, 3H), 3.93 (s, 3H), 3.92 (s, 3H). ¹³C{¹H} NMR (126 MHz, CDCl₃): δ 167.62, 167.30, 166.92, 166.80, 153.29, 148.90, 137.15, 131.63, 130.06, 129.27, 121.86, 120.40, 118.34, 117.22, 113.26, 53.37, 53.34 (two CO₂Me peaks overlapping), 53.19, 29.99. IR (cm⁻¹): 2952, 1732, 1573, 1439, 1397, 1220, 1080, 1020, 1001, 838, 815, 774, 663, 550. HRMS (ESI⁺): 437.0955 calculated for C₂₀H₁₈N₂O₈ [M+Na]⁺, found 437.0946.

### Functionalization of Alternative Ni-bound Heteroaryne Complexes

### General Procedure for Difunctionalization

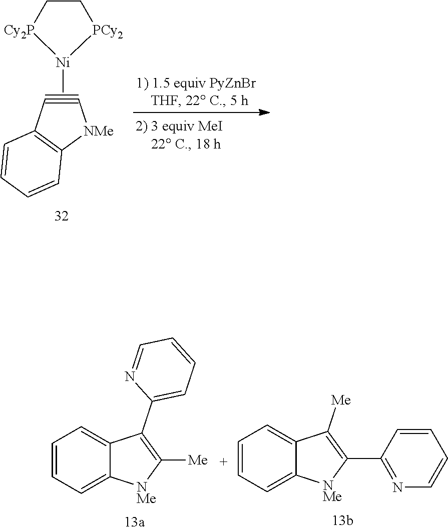

In a glovebox, an oven dried vial was charged with complex 32 (55 mg, 0.90 mmol, 1.0 equiv), and THF (4.5 mL, 0.2 M). 2-pyridyl zinc bromide (0.5 M in THF, 270 μL, 0.135 mmol, 1.5 equiv) was added and the reaction was stirred at 22° C. After 5 hours, methyl iodide (16.8 μL, 0.270 mmol, 3.0 equiv) was added and the reaction was stirred at 22° C. After 18 hours, the vial was taken out of the glovebox. The mixture was diluted with EtOAc (20 mL) and washed with aqueous NH₄Cl solution (20 mL). The organic layer was dried (MgSO₄), filtered, and evaporated to dryness. Purification by preparative TLC (10% EtOAc/90% hexanes, 3 iterations) afforded compound 13a in 50% yield (9.9 mg) and compound 13b in 12% yield (2.4 mg) for a combined 62% yield and 4:1 rr.

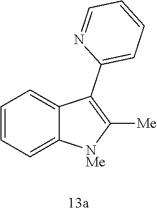 [SMILES: Cc1c(-c2ccccn2)c2ccccc2n1C]

¹H NMR (500 MHz, CDCl₃): δ 8.72 (dd, J=5.1, 1.9 Hz, 1H), 7.88 (d, J=7.9 Hz, 1H), 7.74 (td, J=7.7, 1.9 Hz, 1H), 7.59 (d, J=7.9 Hz, 1H), 7.33 (d, J=8.1 Hz, 1H), 7.25-7.20 (m, 1H), 7.19-7.11 (m, 2H), 3.75 (s, N-Me, 3H), 2.68 (s, C-Me, 3H). ¹³C{¹H} NMR (126 MHz, CDCl₃): δ 155.70, 149.70, 136.93, 136.39, 136.17, 126.69, 123.71, 121.40, 120.33, 120.02, 119.15, 113.40, 108.98, 29.70, 11.61. FTIR (cm⁻¹) 3047, 3013, 2924, 2850, 1712, 1585, 1539, 1475, 1430, 1403, 1369, 1329, 1268, 1214, 1152, 1090, 1023, 1010, 948, 899, 786, 738, 647, 606, 599, 561. HRMS (EI) 221.1073 calculated for C₁₅H₁₄N₂[M]*+, found 221.1064.

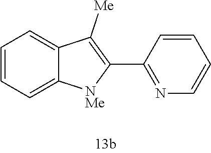 [SMILES: Cc1c(-c2ccccn2)n(C)c2ccccc12]

¹H NMR (500 MHz, CDCl₃):) δ 8.78 (ddd, J=4.9, 2.0, 1.0 Hz, 1H), 7.81 (td, J=7.7, 1.9 Hz, 1H), 7.62 (dt, J=8.0, 1.0 Hz, 1H), 7.51 (dt, J=7.8, 1.1 Hz, 1H), 7.36 (dt, J=8.2, 0.9 Hz, 1H), 7.30-7.27 (m, 1H), 7.15 (ddd, J=7.9, 6.9, 1.0 Hz, 1H), 3.81 (s, N-Me, 3H), 2.41 (s, C-Me, 3H). ¹³C{¹H} NMR (126 MHz, CDCl₃) δ 151.98, 149.78, 138.05, 136.24, 135.93, 128.44, 125.97, 122.67, 121.86, 119.38, 119.24, 110.78, 109.60, 31.45, 9.84. FTIR (cm⁻¹) 3053, 3047, 2926, 2871, 2196, 2158, 2144, 2027, 1998, 1970, 1585, 1564, 1463, 1423, 1355, 1330, 1240, 1050, 1013, 810, 779, 738, 607, 576, 536, 472, 465, 436, 428, 415, 404. HRMS (EI) 222.1157 calculated for C₁₅H₁₄N₂[M], found 222.1168.

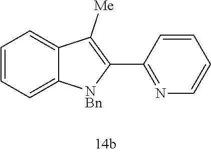 [SMILES: Cc1c(-c2ccccn2)n(Cc2ccccc2)c2ccccc12]

¹H NMR (500 MHz, CDCl₃): δ 8.73 (ddd, J=4.9, 1.9, 1.0 Hz, 1H), 7.71 (td, J=7.7, 1.9 Hz, 1H), 7.64 (dt, J=7.8, 1.1 Hz, 1H), 7.41 (dt, J=7.9, 1.1 Hz, 1H), 7.30-7.27 (m, 2H), 7.24-7.18 (m, 2H), 7.17-7.10 (m, 4H), 6.91 (ddd, J=7.7, 1.7, 0.8 Hz, 2H), 5.59 (s, 2H), 2.42 (s, 3H). ¹³C{¹H} NMR (126 MHz, CDCl₃) δ 151.96, 149.78, 138.90, 137.64, 136.29, 135.71, 128.83, 128.50, 126.98, 126.50, 125.93, 122.86, 121.96, 119.51, 119.46, 111.45, 110.40, 47.67, 9.88. FTIR (cm⁻¹) 3051, 2956, 2925, 2871, 2854, 2196, 2177, 1998, 1585, 1564, 1463, 1383, 1355, 1330, 1240, 1150, 1094, 1068, 1050, 10278, 1013, 987, 922, 822, 810, 779, 738, 607, 436. HRMS (EI) 298.1470 calculated for C₂₁H₁₈N₂[M], found 298.1475.

1. A process for the preparation of a heteroaryne of formula (I): 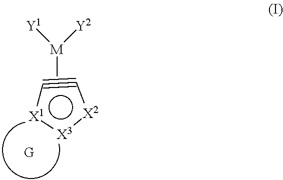
the process comprising the steps of:
(a) converting a compound of formula (II) to a compound of formula (III), and 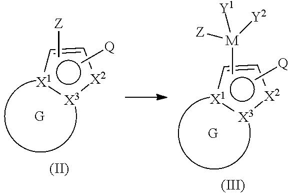
(b) converting the compound of formula (III) to the heteroaryne of formula (I), wherein
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR³R⁴R⁵, —SnR³R⁴R⁵, —BF₃Q⁰, —Li or —Ag,
wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

2. (canceled)

3. The process of claim 1, wherein the heteroaryne has a structure of formula I(a-1): 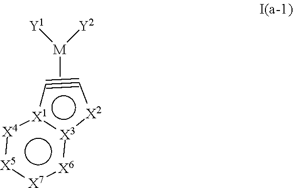
the process comprising the steps of:
(a) converting a compound of formula II(a-1) to a compound of formula III(a-1), and 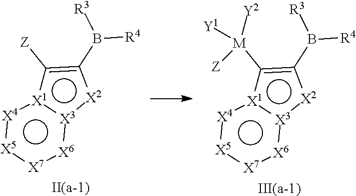
(b) converting the compound of formula III(a-1) to the heteroaryne of formula I(a-1), wherein X⁴, X⁵, X⁶, and X⁷ are independently CH or N.

4. The process of claim 3, wherein X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X¹ is C, X² is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH;
or wherein X⁷ is N, and
(xiii) X¹ is C, X² is NR², X³ is C, X⁴ is CH, X⁵ is CH, and X⁶ is CH.

5. (canceled)

6. The process of claim 1, wherein Z is halogen.

7. (canceled)

8. (canceled)

9. The process of claim 1, wherein Q is —BR³R⁴, wherein R³ and R⁴ are independently hydroxy or alkoxy, or R³ and R⁴ together with the boron that they are attached to form a 5-, 6-, or 7-membered ring optionally substituted with one or more alkyl groups.

10. (canceled)

11. The process of claim 1, wherein M is Nickel or Palladium.

12. (canceled)

13. (canceled)

14. The process of claim 1, wherein -M(Y¹)(Y²)

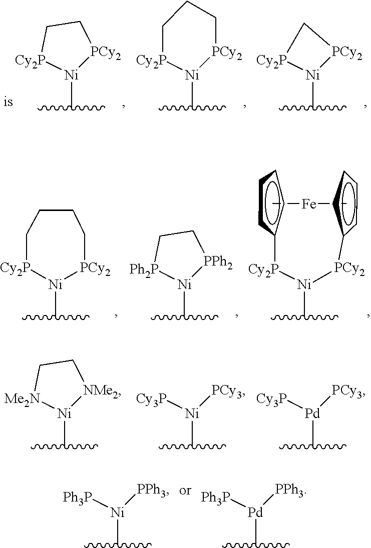

15. The process of claim 1, wherein the compound of formula (II), formula II(a), or formula II(a-1) is

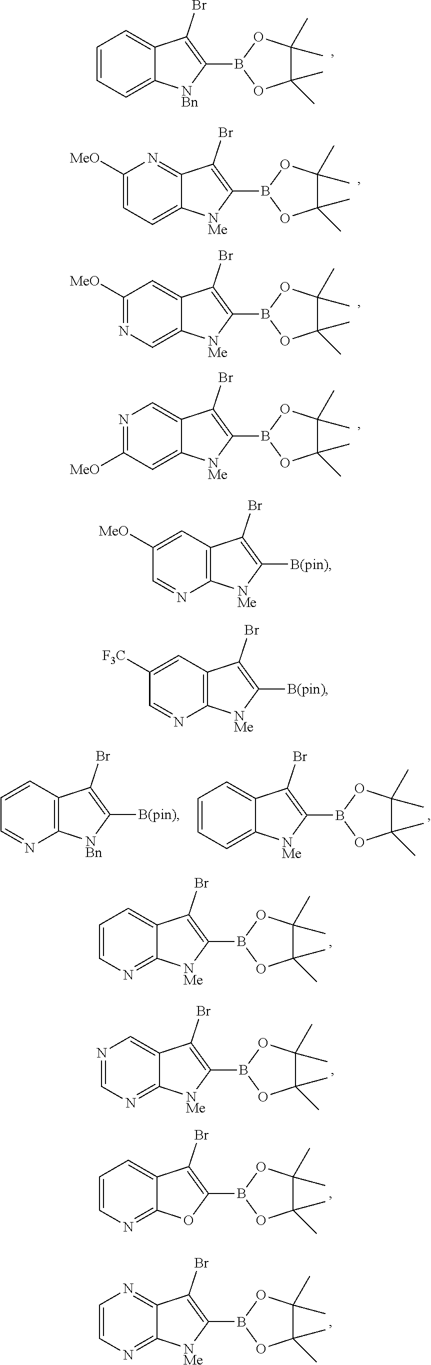

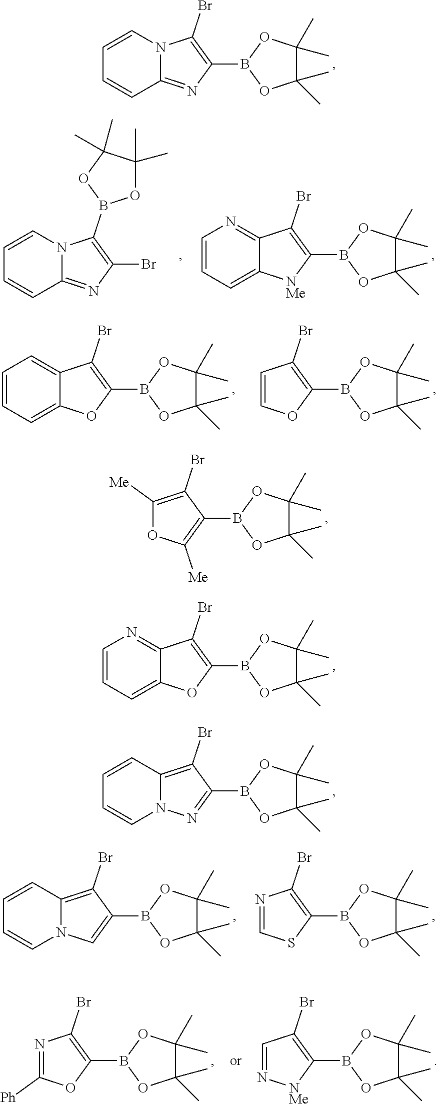

16-21. (canceled)

22. The process of claim 1, further comprising converting a compound of formula (IV) to the compound of formula (II)

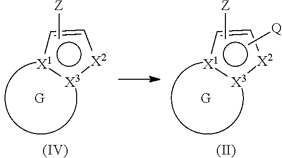

23. (canceled)

24. The process of claim 22, comprising converting a compound of formula IV(a-1) to the compound of formula II(a-1).

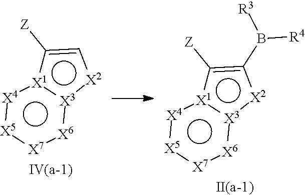

25. A compound of Formula (II), or a salt thereof: 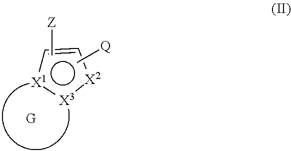
wherein:
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, haloalkyl, alkoxy, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups; and
Z is halogen, —OSO₂R⁶, or —OC(O)R⁶, wherein R⁶ is alkyl, haloalkyl, aryl or amino.

26. (canceled)

27. The compound of claim 25, wherein the compound has a structure of Formula II(a-1): 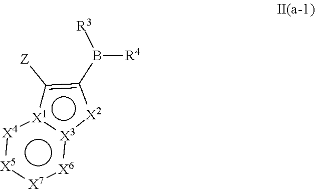
wherein X⁴, X⁵, X⁶, and X⁷ are independently CH or N.

28. The compound of claim 27, wherein X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH;
or wherein X⁷ is N, and
(xiii) X¹ is C, X² is NR², X³ is C, X⁴ is CH, X⁵ is CH, and X⁶ is CH.

29-34. (canceled)

35. The compound of claim 25, wherein the compound is

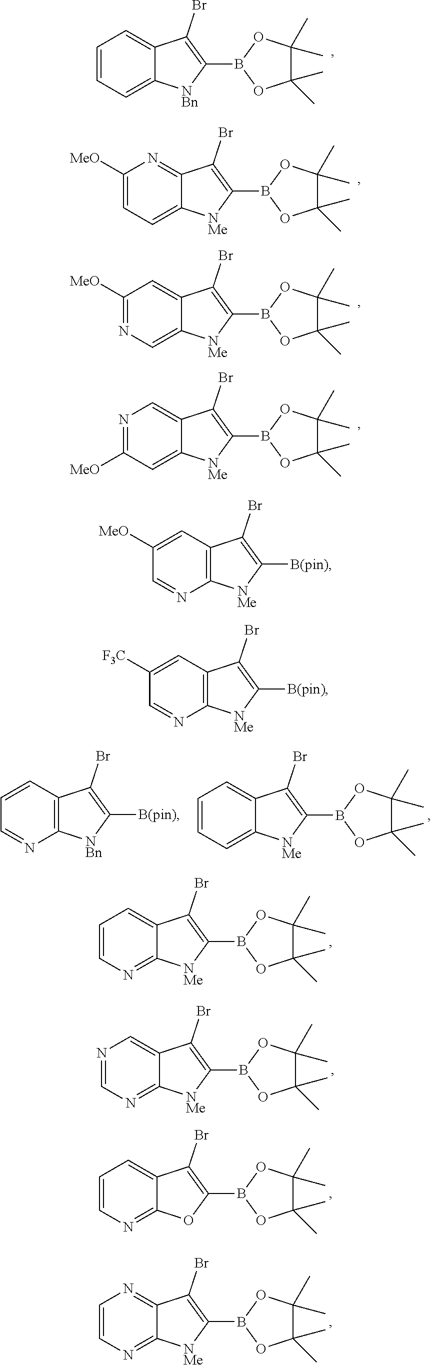

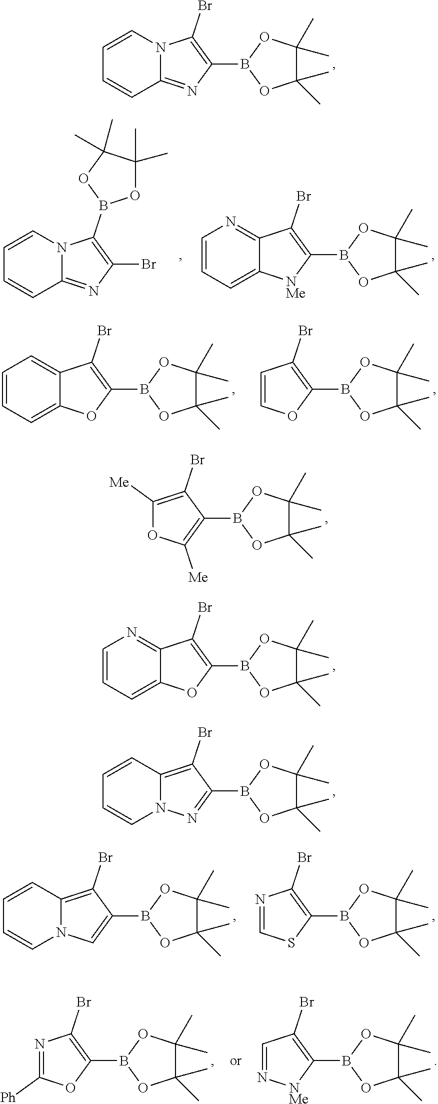

36. A compound of Formula (III), or a salt thereof: 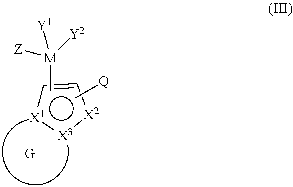
wherein:
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

37. (canceled)

38. The compound of claim 36, wherein the compound has a structure of Formula III(a-1): 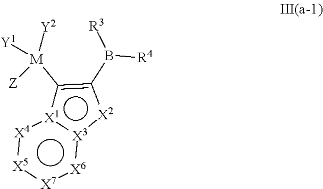
wherein X⁴, X⁵, X⁶, and X⁷ are independently CH or N.

39. The compound of claim 38, wherein X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH;
or wherein X⁷ is N, and
(xiii) X¹ is C, X² is NR², X³ is C, X⁴ is CH, X⁵ is CH, and X⁶ is CH.

40-49. (canceled)

50. The compound of claim 36, wherein the compound is

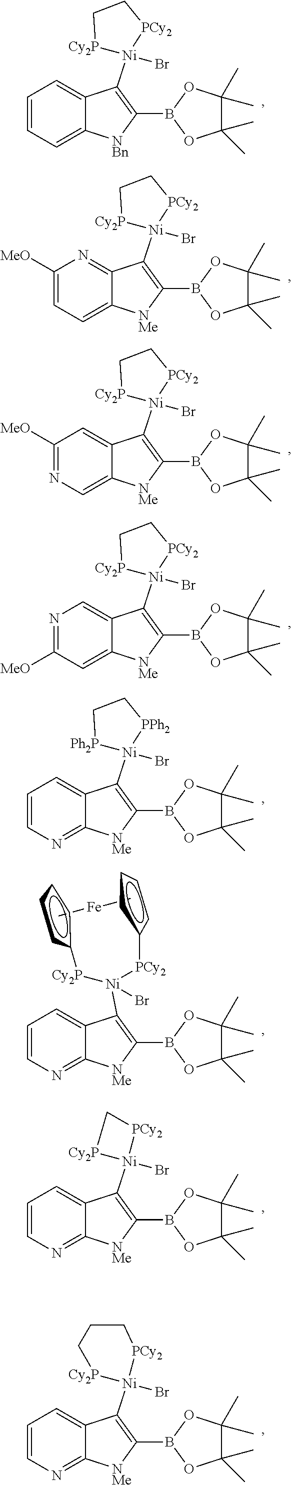

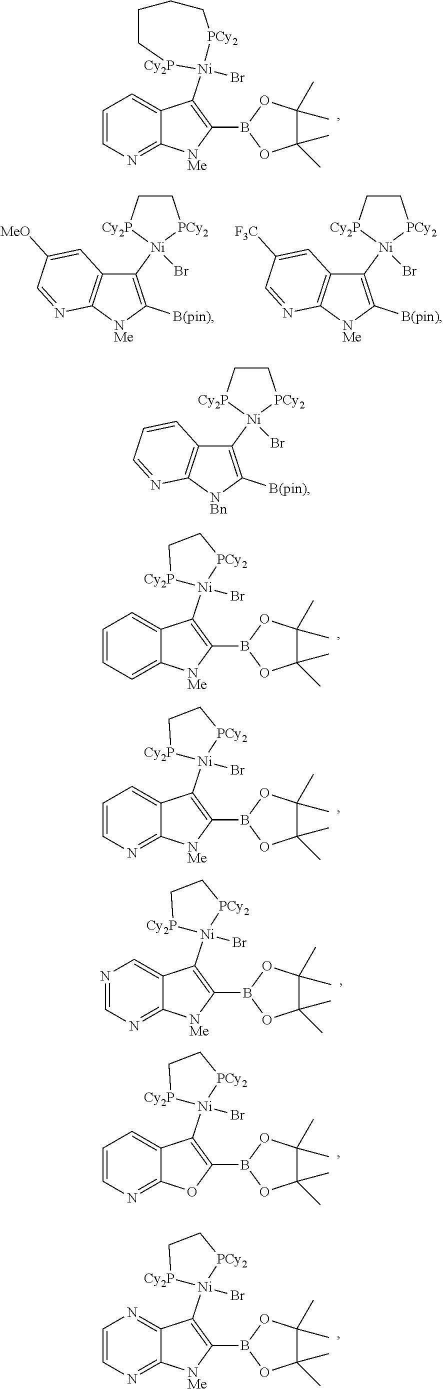

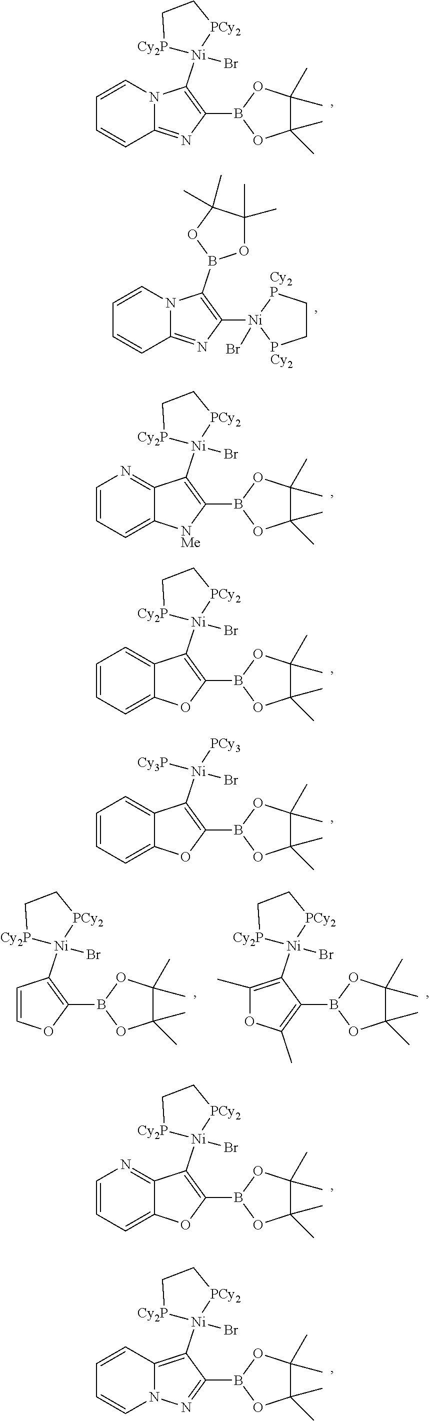

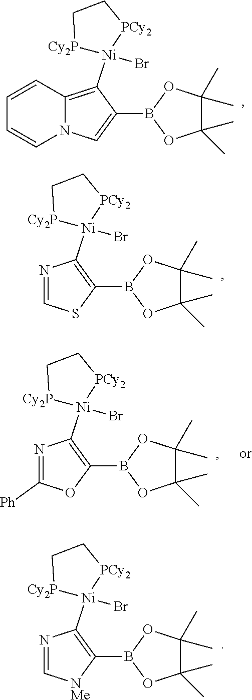

51. A heteroaryne of Formula (I), or a salt thereof: 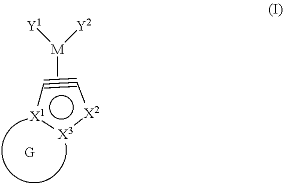
wherein
X¹, X², and X³ are independently C, CR¹, N, NR², S or O;
R¹ and R² at each occurrence are independently H, alkyl, alkoxy, aryl; —C₁₋₄alkylene-aryl, —SiR^{A}R^{B}R^{C}, —SO₂R^{A}, —C(O)OR^{A}, —C(O)NR^{A}R^{B}, or —C(O)R^{A};
G is an optional 5- to 8-membered aromatic ring, wherein G is optionally substituted with one or more substituents, each of which being independently alkyl, halo, alkoxy, haloalkyl, aryl, amino, nitro, cyano, —SiR^{A}R^{B}R^{C}, —SO₂R^{A}, —C(O)OR^{A}, —C(O)R^{A}, or —C(O)NR^{A}R^{B};
R^{A}, R^{B}, and R^{C} at each occurrence are independently H, alkyl, haloalkyl, or aryl;
Q is —BR³R⁴, —ZnR³, —MgR³, —SiR₃R⁴R⁵, —SnR₃R⁴R⁵, —BF₃Q⁰, —Li, or —Ag,
wherein Q⁰ is a counter ion, R³, R⁴ and R⁵ are independently H, alkyl, hydroxy, alkoxy, halogen, or aryl, or R³ and R⁴ together with the boron atom they are attached to form a 5-, 6-, or 7-membered ring optionally substituted with one or more alkyl groups;
M is a transition metal;
Z is halogen, —OSO₂R⁶, or —OC(O)R⁶, wherein R⁶ is alkyl, haloalkyl, aryl or amino; and
Y¹ and Y² are independently —N(R⁷)₂, —P(R⁷)₃, or Y¹ and Y² are joined to form —N(R⁷)—(CR⁸R⁹)ₙ—N(R⁷)—, —N(R⁷)₂—(CR⁸R⁹)ₙ—N(R⁷)₂—, —P(R⁷)₂—(CR⁸R⁹)ₙ—P(R⁷)₂—, or —P(R⁷)₂-L-P(R⁷)₂—, wherein R⁷ at each occurrence is an alkyl, aryl, or cycloalkyl, R⁸ and R⁹ are independently H, aryl or alkyl, L is a metallocene, and n is 1, 2, 3, or 4.

52. The heteroaryne of claim 51, wherein the heteroaryne has a structure of Formula I(a-1): 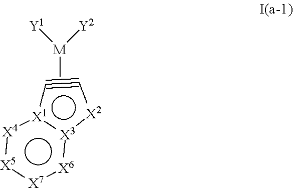
wherein X⁴, X⁵, X⁶, and X⁷ are independently CH or N.

53. The heteroaryne of claim 52, wherein X⁷ is CH, and
(i) X¹ is C, X² is NR², X³ is C, X⁴ is CH, X⁵ is CH, and X⁶ is N,
(ii) X¹ is C, X² is NR², X³ is C, X⁴ is CH, X⁵ is N, and X⁶ is N,
(iii) X¹ is C, X² is O, X³ is C, X⁴ is CH, X⁵ is CH, and X⁶ is N,
(iv) X¹ is C, X² is NR², X³ is C, X⁴ is N, X⁵ is CH, and X⁶ is N,
(v) X¹ is N, X² is N, X³ is C, X⁴ is CH, X⁵ is CH, and X⁶ is CH,
(vi) X¹ is C, X² is O, X³ is C, X⁴ is CH, X⁵ is CH, and X⁶ is CH,
(vii) X¹ is C, X² is NR², X³ is C, X⁴ is CH, X⁵ is CH, and X⁶ is CH,
(viii) X1 is C, X2 is NR², X³ is C, X⁴ is N, X⁵ is CH, and X⁶ is CH,
(ix) X¹ is C, X² is O, X³ is C, X⁴ is N, X⁵ is CH, and X⁶ is CH,
(x) X¹ is C, X² is N, X³ is N, X⁴ is CH, X⁵ is CH, and X⁶ is CH,
(xi) X¹ is C, X² is CH, X³ is N, X⁴ is CH, X⁵ is CH, and X⁶ is CH, or
(xii) X¹ is C, X² is NR², X³ is C, X⁴ is CH, X⁵ is N, and X⁶ is CH;
or wherein X⁷ is N, and
(xiii) X¹ is C, X² is NR², X³ is C, X⁴ is CH, X⁵ is CH, and X⁶ is CH.

54-58. (canceled)

59. The heteroaryne of claim 51, wherein the heteroaryne is

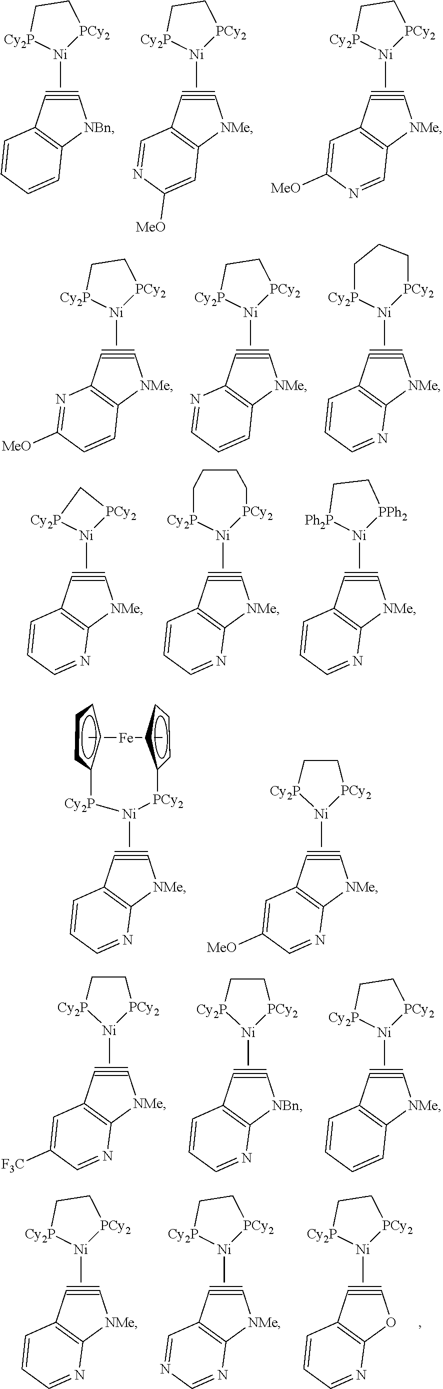

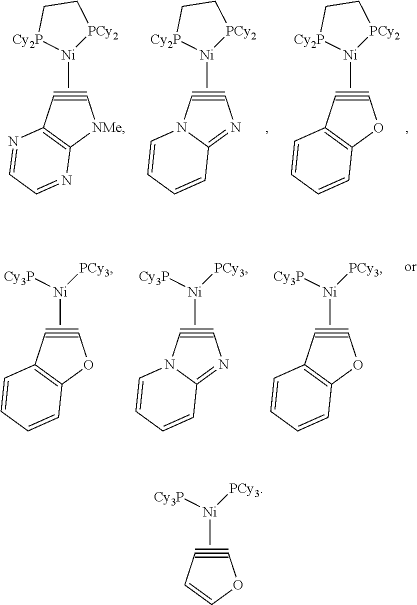

60. A method for preparing a target molecule, comprising coupling the compound of claim 51 with a partner molecule, thereby producing the target molecule.

61. The method of claim 60, wherein the target molecule is 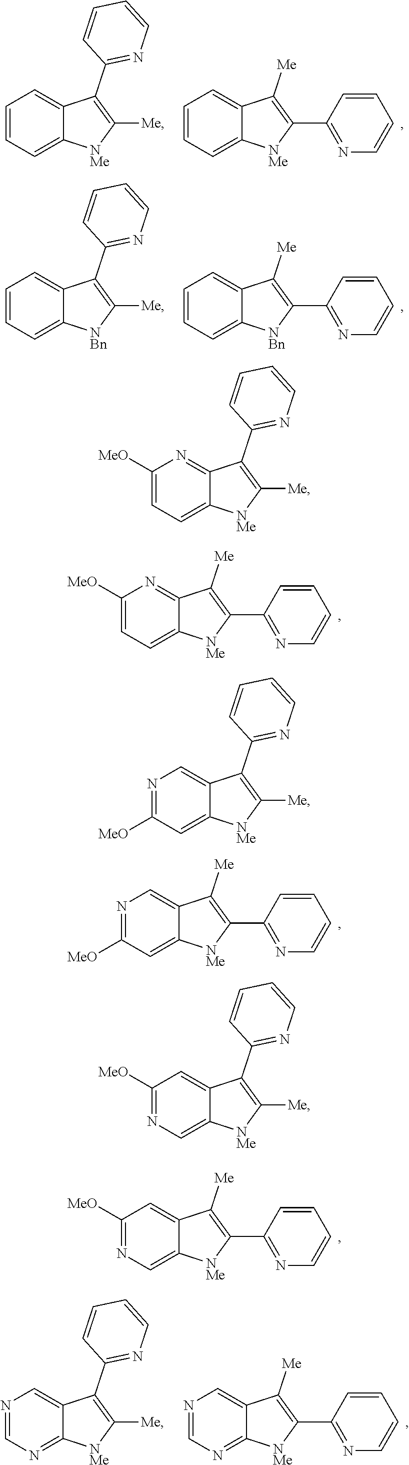 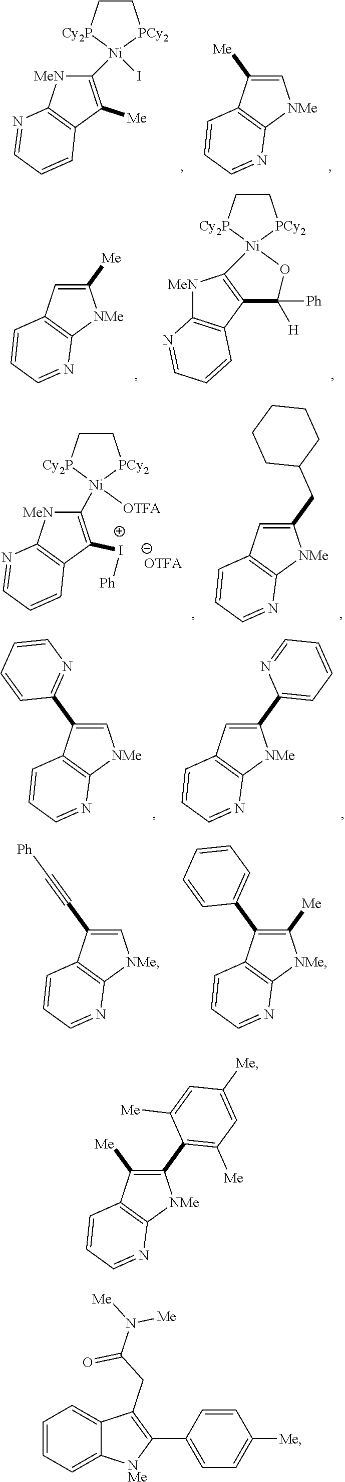 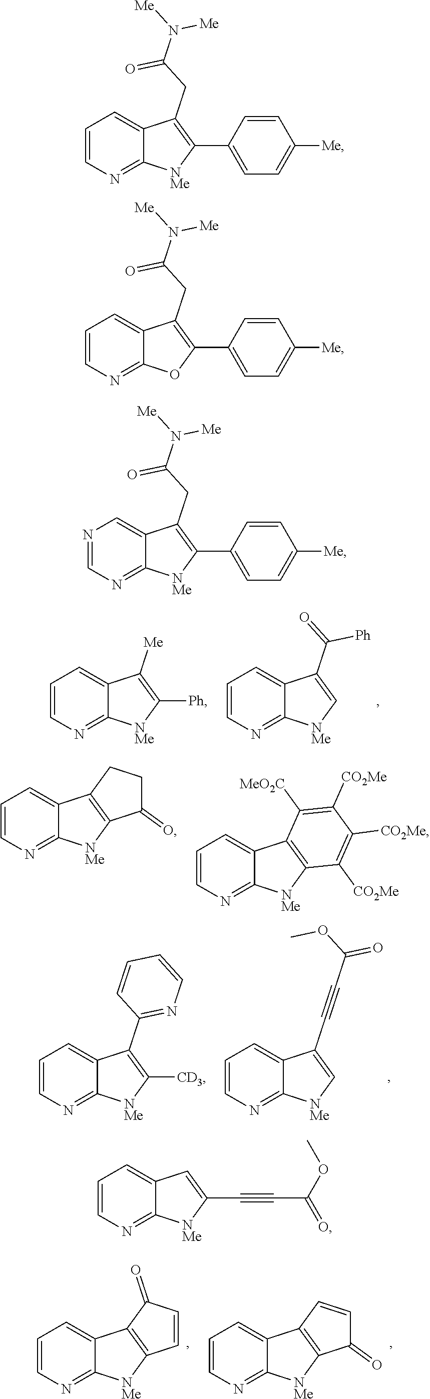 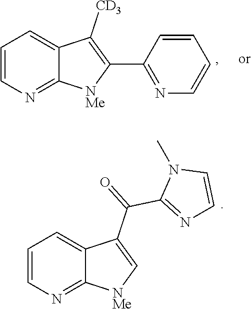 [SMILES: [2H]C([2H])([2H])c1c(-c2ccccn2)n(C)c2ncccc12 ; Cn1ccnc1C(=O)c1cn(C)c2ncccc12]
